(19) United States
(12) Patent Application Publication
(10) Pub. No.: US 2005/0202424 A1
(43) Pub. Date: Sep. 15, 2005

(54) Regulators of biofilm formation and uses thereof

(76) Inventors: Frederick M Ausubel, NEWTON, MA (US); Ella Drankard, Cambridge, MA (US)

Correspondence Address: CLARK & ELBING LLP, 101 FEDERAL STREET, BOSTON, MA, 02110, US

(21) Appl. No.: 10/482,948

(22) PCT Filed: Jul. 8, 2002
(86) PCT No.: PCT/US02/21431

§ 371 Date: Nov. 29, 2004

Related U.S. Application Data
(60) Provisional application No. 60/303,286, filed on Jul. 6, 2001.
(60) Provisional application No. 60/373,233, filed on Apr. 16, 2002.

(51) Int. Cl. C12Q 1/68; C07H 21/04; C12N 1/21; C12Q 1/18; C07K 14/195; C12N 15/74

(52) U.S. Cl. 435/6; 435/32; 435/69.1; 435/471; 435/252.3; 530/350; 536/23.7

(57) ABSTRACT

This invention relates to nucleic acid and amino acid sequences of genes regulating bacterial biofilm formation and to the use of these sequences as targets in the diagnosis, treatment, and prevention of bacterial infection and pathogenesis. In addition, the invention relates to screening methods for identifying modulators of bacterial biofilm formation and the development of antibacterial treatments.

This application claims benefit of U.S. provisional application 60/303,286 and 60/373,233, filed Jul. 6, 2001 and Apr. 16, 2002, respectively. The disclosures of which are hereby incorporated by reference.

### BACKGROUND OF THE INVENTION

This invention relates to nucleic acid and amino acid sequences of genes regulating bacterial biofilm formation and to the use of these sequences as targets in the diagnosis, treatment, and prevention of bacterial infection and pathogenesis. In addition, the invention relates to screening methods for identifying modulators of bacterial biofilm formation and the development of antibacterial treatments.

Bacteria possess the ability to form aggregated, organized, colonial communities called biofilms. Distinct from their free-living planktonic counterparts, bacterial cells that form biofilms secrete an exopolysacharide slime that surrounds and protects the bacterial colony. By adhering to each other and to surfaces or interfaces, these matrix-enclosed bacterial populations can cause any number of problems. By attaching to a variety of surfaces such as contact lenses, water pipes, hip replacements and food packaging, they can cause irritation, disease, immune rejection, and food poisoning.

In addition to attaching to abiotic surfaces, many biofilm-forming bacteria colonize living tissue where they cause serious infection. For example, Pseudomonas aeruginosa colonizes the lungs of cystic fibrosis (CF) patients as a biofilm. Chronic colonization of the airways by this bacterial pathogen leads to debilitating exacerbation of pulmonary infection and constitutes the major cause of morbidity and mortality in CF populations. Colonization of the CF lung by P. aeruginosa generally persists despite the use of long-term antibiotic therapy, since antibiotic treatment rarely results in complete eradication of the infection.

As current antibiotic therapies offer limited effectiveness in treating biofilm infection, a need exists for developing therapeutic agents that prevent biofilm formation. The discovery of polypeptides that regulate biofilm formation and polynucleotides encoding such polypeptides fulfills a need in the art by providing new compositions that are useful in the diagnosis, treatment, and prevention of bacterial infection and pathogenesis, as well as biofilm formation in both industrial and medical settings.

### SUMMARY OF THE INVENTION

As is described in more detail below, we have discovered a regulatory system that modulates microbial phenotypic switching. In one aspect, the invention features an isolated polypeptide that includes an amino acid sequence that is at least 50% (and preferably 60%, 65%, 70%, 75%, 80%, 85%, 90%, or 95-99%) identical to the amino acid sequence of PvrR (SEQ ID NO:2), wherein expression of the polypeptide, in a microorganism, affects phenotype-mediated antibiotic-resistance in the microorganism. In preferred embodiments, the polypeptide includes the amino acid sequence of PvrR (SEQ ID NO:2) or consists essentially of the amino acid sequence of PvrR (SEQ ID NO:2) or a fragment thereof.

In a related aspect, the invention features an isolated polypeptide fragment of an isolated polypeptide that includes an amino acid sequence having at least 50% identity to the amino acid sequence of PvrR (SEQ ID NO:2). In preferred embodiments, such a polypeptide fragment includes at least 300 contiguous amino acid residues of the amino acid sequence of PvrR (SEQ ID NO:2). In other embodiments, the fragment is at least 250 amino acid residues, 200 amino acid residues, or 100 amino acid residues of the amino acid sequence of PvrR (SEQ ID NO:2).

In another aspect, the invention features an isolated polynucleotide having at least 50% identity to the nucleotide sequence of pvrR (SEQ ID NO:1), wherein expression of the polynucleotide, in a microorganism, affects phenotype-mediated antibiotic-resistance in the microorganism. In preferred embodiments, the isolated polynucleotide includes the nucleotide sequence of pvrR (SEQ ID NO:1) or a complement thereof. In yet other preferred embodiments, the polynucleotide consists essentially of the nucleotide sequence of pvrR (SEQ ID NO:1) or a fragment thereof.

In still other related aspects, the invention features a vector including any of the aforementioned isolated polynucleotides and a host cell that includes the vector.

The invention further features a variety of screening assays for identifying compounds that modulate phenotype-mediated antibiotic-resistance, biofilm formation, or biofilm-mediated antibiotic resistance. For example, the invention features a screening method that is useful for identifying a compound that modulates the gene expression of a regulator polynucleotide that affects phenotype-mediated antibiotic-resistance in a microorganism. Such a method includes the steps of: (a) providing a microbial cell (e.g., Pseudomonas, Vibrio, Salmonella, or Staphylococcus) that includes a polynucleotide having at least 50% identity to the nucleotide sequence of pvrR (SEQ ID NO:1) (or a nucleotide sequence that is substantially identical to pvrR), wherein expression of the polynucleotide, in the microbial cell, affects phenotype-mediated antibiotic-resistance in the microbial cell; (b) contacting the microbial cell with a compound; and (c) comparing the level of gene expression of the polynucleotide in the presence of the compound with the level of gene expression in the absence of the compound; wherein a measurable difference in gene expression indicates that the compound modulates gene expression of a regulator polynucleotide that affects phenotype-mediated antibiotic-resistance in a microorganism.

In preferred embodiments, the screening method identifies a compound that increases or decreases transcription of the regulator polynucleotide. In other embodiments, the screening method identifies a compound that increases or decreases translation of an mRNA transcribed from the regulator polynucleotide.

In other preferred embodiments, the microbial cell is a phenotypic variant (e.g., a small colony variant) having increased biofilm formation. Preferably, the small colony variant is a small colony variant of Pseudomonas, Vibrio, Salmonella, or Staphylococcus. In still other embodiments, the small colony variant is a rough small colony variant, for example, a rough small colony variant of Pseudomonas, Vibrio, Salmonella, or Staphylococcus. In a preferred embodiment, the rough small colony variant is Pseudomonas aeruginosa PA14 RSCV.

In other preferred embodiments, the activity of the compound used in the screening assay is dependent upon the presence of the pvrR gene (SEQ ID NO:1) or a functional equivalent thereof. For example, the identified compound targets and interacts with the pvrR gene (SEQ ID NO:1) or a functional equivalent thereof. In still other preferred embodiments, the expression of the regulator polynucleotide mediates phenotypic switching of the microbial cell in the presence of a high concentration of an antibiotic. In other preferred embodiments of the screening method, the polypeptide is expressed using an isolated polynucleotide that expresses a polypeptide having an amino acid sequence having at least 50% identity to the amino acid sequence of PvrR (SEQ ID NO:2) or a fragment thereof.

In another aspect, the invention features a screening method for identifying a compound that modulates an activity of a polypeptide that affects phenotype-mediated antibiotic-resistance in a microorganism. The method, in general, includes the steps of: (a) providing a microbial cell expressing a polypeptide having at least 50% identity to the amino acid sequence of PvrR (SEQ ID NO:2) (or a polypeptide that is substantially identical to PvrR), wherein expression of the polypeptide, in the microbial cell, affects phenotype-mediated antibiotic-resistance in the microbial cell; (b) contacting the microbial cell with a compound; and (c) comparing an activity of the polypeptide in the presence of the compound with the activity in the absence of the compound; wherein a measurable difference in the activity indicates that the compound modulates the activity of the polypeptide that affects phenotype-mediated antibiotic-resistance in a microorganism. In preferred embodiments, the screening method identifies a compound that increases or decreases the activity of the polypeptide. Comparison of the activity of the polypeptide includes a variety of standard biochemical analyses including immunological assays.

In preferred embodiments, the microbial cell utilized in the screening assay is a phenotypic variant (e.g., Pseudomonas aeruginosa PA14 RSCV) having increased biofilm formation relative to wild-type.

In other preferred embodiments, the regulator polypeptide is an isolated polypeptide that includes an amino acid sequence having at least 50% identity to the amino acid sequence of PvrR (SEQ ID NO:2) (or a polypeptide that is substantially identical to PvrR). In particular, such a polypeptide has the ability to regulate phenotypic switching; to regulate biofilm-mediated antibiotic-resistance; to mediate phenotypic switching of the microbial cell in the presence of a high concentration of an antibiotic; or to affect susceptibility of the microbial cell to antibiotic treatment; or to regulate, or mediate, or affect, or any combination of the aforementioned activities thereof. In other preferred embodiments, the regulator polypeptide is an element of a two-component regulatory system. In yet other preferred embodiments, the polypeptide is expressed by an isolated polynucleotide having at least 50% identity to the nucleotide sequence of pvrR (SEQ ID NO:1) or a fragment thereof.

Typically, the activity of the compound identified in the screening assay is dependent upon the presence of the PvrR polypeptide (SEQ ID NO:2) or a functional equivalent thereof. In particular aspects of the screening assay, the compound targets the PvrR polypeptide (SEQ ID NO:2) or a functional equivalent thereof.

In another aspect, the invention features a screening method for identifying a compound that modulates microbial biofilm formation. This method, in general, includes the steps of: (a) culturing a microbial cell (e.g., Pseudomonas, Vibrio, Salmonella, or Staphylococcus) that includes a polypeptide having at least 50% identity to the amino acid sequence of PvrR (SEQ ID NO:2) (or a polypeptide that is substantially identical to PvrR), wherein the microbial cell, upon culturing, forms a biofilm; (b) contacting the microbial cell with a compound; and (c) comparing microbial biofilm formation in the presence of the compound with microbial biofilm formation in the absence of the compound; wherein a measurable difference in the microbial biofilm formation indicates that the compound modulates biofilm formation.

In preferred embodiments, the screening method identifies a compound that increases or decreases biofilm formation. Typically, such biofilm formation is measured by using any standard method, for example, by assaying microbial aggregation (e.g., by using a microscope); using a salt aggregation test; or by using an attachment assay.

In preferred embodiments, the microbial cell is a phenotypic variant having increased biofilm formation when compared to its wild-type such as a small colony variant of Pseudomonas, Vibrio, Salmonella, or Staphylococcus. In other preferred embodiments, the small colony variant is a rough small colony variant of Pseudomonas, Vibrio, or Salmonella. In a preferred embodiment, the rough small colony variant is Pseudomonas aeruginosa PA14 RSCV.

In yet other preferred embodiments, the activity of the compound utilized in the screening assay is dependent upon the presence of PvrR polypeptide (SEQ ID NO: 2) or a functional equivalent thereof. For example, the identified compound targets and interacts with the PvrR polypeptide (SEQ ID NO:2) or a functional equivalent thereof, resulting in increasing or decreasing its functional activity.

In still another embodiment, the expression of the polypeptide mediates phenotypic switching of the microbial cell in the presence of a high concentration of an antibiotic.

In another embodiment, the polypeptide is an isolated polypeptide that includes an amino acid sequence having at least 50% identity to the amino acid sequence of PvrR (SEQ ID NO:2), wherein expression of the polypeptide, in a microorganism, affects phenotype-mediated antibiotic-resistance in the microorganism.

In still another aspect, the invention features a method of treating a microbial infection involving a microorganism that forms a biofilm in a mammal. The method, in general, includes administering to the mammal a therapeutically-effective amount of a compound that induces or represses expression or activity of a polypeptide that includes an amino acid sequence having at least 50% identity to the amino acid sequence of PvrR (SEQ ID NO:2) (or a polypeptide that is substantially identical to PvrR) or a fragment thereof, wherein expression of the polypeptide or the fragment thereof, in a microorganism, affects phenotype-mediated antibiotic-resistance in the microorganism.

In preferred embodiments, the method further includes administering to the mammal a therapeutically-effective amount of an antibiotic. The treatment is particularly useful for treating patients having cystic fibrosis or a chronic microbial infection or both. In other preferred embodiments, the microorganism treated using the method belongs to the genus Pseudomonas, Vibrio, Salmonella, or Staphylococcus.

In yet another aspect, the invention features a method of cleaning, disinfecting, or decontaminating a surface at least partially covered by a microorganism that forms a biofilm, the method involving contacting the microorganism with a cleaning composition including a compound that induces or represses expression or activity of a polypeptide that includes an amino acid sequence having at least 50% identity to the amino acid sequence of PvrR (SEQ ID NO:2) (or a polypeptide that is substantially identical to PvrR) or fragment thereof, wherein expression of the polypeptide or the fragment thereof, in a microorganism, affects phenotype-mediated antibiotic-resistance in the microorganism.

In yet another aspect, the invention features a screening method for identifying a compound that decreases pathogenicity of an antibiotic-resistant phenotypic variant. The method, in general, includes the steps of: (a) contacting an antibiotic-resistant phenotypic variant with a candidate compound; and (b) measuring reversion of the antibiotic-resistant phenotypic variant to a wild-type phenotype, an increase in reversion indicating that the compound decreases pathogenicity of the antibiotic-resistant phenotypic variant. In preferred embodiments, the antibiotic-resistant phenotypic variant is cultured in the absence of an antibiotic, has increased biofilm formation; is a rough small colony variant; is a hyperpiliated variant; has increased hydrophobicity; has an alteration in a surface component; or is a pathogen such as a Gram positive bacterium (e.g., Staphylococcus) or a Gram negative bacterium (e.g., Vibrio, Pseudomonas, or Salmonella).

In another aspect, the invention features a screening method for identifying a compound that decreases pathogenicity of an antibiotic-resistant phenotypic variant. The method, in general, includes the steps of: (a) culturing an antibiotic-resistant phenotypic variant with a candidate compound in the presence of an antibiotic; and (b) comparing the number of antibiotic-resistant phenotypic variants in the presence of the compound to the number of antibiotic-resistant phenotypic variants in the absence of the compound, a decrease in the number of the antibiotic-resistant phenotypic variants in the presence of the compound indicating that the compound decreases pathogenicity of the antibiotic-resistant phenotypic variant.

In yet another aspect, the invention features a screening method for identifying a polynucleotide encoding a regulator polypeptide, the method including the steps of: (a) providing a mutagenized microbe; (b) culturing the mutagenized microbe in the presence of an antibiotic; and (c) comparing the mutagenized microbe with a control wild-type microbe, wherein a change in the number of phenotypic variants identifies the mutagenized microbe as having a mutation in a polynucleotide encoding a regulator polypeptide. In preferred embodiments, the phenotypic variant is a small colony variant.

In another aspect, the invention features a screening method for identifying a polynucleotide encoding a regulator polypeptide that modulates an antibiotic-resistant phenotype of a microorganism. The method, in general, includes the steps of: (a) identifying an antibiotic-resistant phenotypic variant of a microorganism including a first phenotype; (b) mutagenizing the antibiotic-resistant phenotypic variant of the microorganism, thereby generating a mutated phenotypic variant of the microorganism; and (c) selecting the mutated phenotypic variant of step (b) having a second phenotype, other than the first phenotype of the antibiotic-resistant phenotypic variant, wherein the second phenotype identifies a mutation in the mutated phenotypic variant of step (b); and (d) using the mutation for identifying a polynucleotide encoding a regulator polypeptide that modulates an antibiotic-resistant phenotype of a microorganism. In preferred embodiments, the second phenotype includes a wild-type phenotype.

In yet another aspect, the invention features a screening method for identifying a polynucleotide encoding a regulator polypeptide that modulates phenotype-mediated antibiotic-resistance of a microorganism. The method, in general, includes the steps of: (a) transforming an antibiotic-resistant phenotypic variant of a microorganism with a candidate polynucleotide encoding a regulator polypeptide; and (b) culturing the transformed antibiotic-resistant phenotypic variant of a microorganism under conditions suitable for expression of the regulator polypeptide; and (c) measuring reversion of the transformed antibiotic-resistant phenotypic variant of the microorganism to a wild-type phenotype, an increase in reversion identifies the polynucleotide as encoding a regulator polypeptide that modulates phenotype-mediated antibiotic-resistance.

In preferred embodiments, the polynucleotide encodes a regulator polypeptide that modulates a phenotypic switch from an antibiotic-resistant phenotype to an antibiotic-susceptible phenotype. In other preferred embodiments, the candidate polynucleotide has at least 50% identity to the nucleotide sequence of pvrR (SEQ ID NO:1) (or a polynucleotide sequence that is substantially identical to pvrR). In other embodiments, the candidate polynucleotide sequence is substantially identical to any one of the polynucleotides shown in FIGS. 5B, 5C, 6A-6K, and 7A-7E. In other preferred embodiments, the candidate polynucleotide encodes a polypeptide that is an element of a two-component regulatory system.

In another aspect, the invention features an isolated polypeptide including an amino acid sequence that is substantially identical to the amino acid sequence of any one the polypeptides shown in FIGS. 5E (SEQ ID NO: 4) and 6L-6V (SEQ ID NOS: 19-29), each of which are encoded by a polynucleotide of the ORF1 region.

For example, with respect to the ORF1 region, the invention features an isolated polypeptide that includes an amino acid sequence that is at least 50% (and preferably 60%, 65%, 70%, 75%, 80%, 85%, 90%, or 95-99%) identical to the amino acid sequence of the polypeptide shown in FIG. 5E (SEQ ID NO: 4) or to a polypeptide shown in FIGS. 6L-6V (SEQ ID NOS: 19-29), wherein expression of the polypeptide, in a microorganism, affects phenotype-mediated antibiotic-resistance in the microorganism. Preferably, the polypeptide includes the amino acid sequence shown in FIG. 5E or consists essentially of the amino acid sequence shown in FIG. 5E or a fragment thereof.

In a related aspect, the invention features an isolated polypeptide fragment of an isolated polypeptide that includes an amino acid sequence having at least 50% identity to the amino acid sequence the polypeptide shown in FIG. 5E or to a polypeptide shown in any one of FIGS. 6L-6V. In preferred embodiments, such a polypeptide fragment includes at least 400 contiguous amino acid residues of the amino acid sequence shown in any one of FIGS. 5E and 6L-6V. In other embodiments, the fragment is at least 300 amino acid residues, 200 amino acid residues, or 100 amino acid residues of the polypeptides shown in FIGS. 5E and 6L-6V.

In another aspect, the invention features an isolated polynucleotide molecule including a sequence substantially identical to any one of the polynucleotides shown in FIGS. 5B (SEQ ID NO:3) and 6A-6K (SEQ ID NOS: 8-18), which are found in the ORF1 region. In preferred embodiments, the isolated polynucleotide molecule has at least 45%, 50%, 60%, 70%, 80%, 90%, or even 95-99% identity to any one of these isolated molecules.

For example, with respect to the ORF1 region, the invention features an isolated polynucleotide having at least 50% identity to the nucleotide sequence shown in FIG. 5B or to any one of the nucleotide sequences shown in FIGS. 6A-6K, wherein expression of the polynucleotide, in a microorganism, affects phenotype-mediated antibiotic-resistance in the microorganism. In preferred embodiments, the isolated polynucleotide includes the nucleotide sequence shown in FIG. 5B or a complement thereof. In yet other preferred embodiments, the polynucleotide consists essentially of the nucleotide sequence shown in FIG. 5B or a fragment thereof.

In still other related aspects, the invention features a vector including any of the aforementioned isolated polynucleotides and a host cell that includes the vector.

The invention further features a variety of screening assays for identifying compounds that modulate phenotype-mediated antibiotic-resistance, biofilm formation, or biofilm-mediated antibiotic resistance. For example, the invention features a screening method that is useful for identifying a compound that modulates the gene expression of a regulator polynucleotide that affects phenotype-mediated antibiotic-resistance in a microorganism. Such a method includes the steps of: (a) providing a microbial cell (e.g., Pseudomonas, Vibrio, Salmonella, or Staphylococcus) that includes a polynucleotide that is substantially identical to any one of the nucleotide sequences shown in FIG. 5B or 6A-6K (or a polynucleotide having at least 40% identity to any one of these sequences), wherein expression of the polynucleotide, in the microbial cell, affects phenotype-mediated antibiotic-resistance in the microbial cell; (b) contacting the microbial cell with a compound; and (c) comparing the level of gene expression of the polynucleotide in the presence of the compound with the level of gene expression in the absence of the compound; wherein a measurable difference in gene expression indicates that the compound modulates gene expression of a regulator polynucleotide that affects phenotype-mediated antibiotic-resistance in a microorganism.

In preferred embodiments, the screening method identifies a compound that increases or decreases transcription of the regulator polynucleotide. In other embodiments, the screening method identifies a compound that increases or decreases translation of an mRNA transcribed from the regulator polynucleotide.

In other preferred embodiments, the microbial cell is a phenotypic variant (e.g., a small colony variant) having increased biofilm formation. Preferably, the small colony variant is a small colony variant of Pseudomonas, Vibrio, Salmonella, or Staphylococcus. In still other embodiments, the small colony variant is a rough small colony variant, for example, a rough small colony variant of Pseudomonas, Vibrio, Salmonella, or Staphylococcus. In a preferred embodiment, the rough small colony variant is Pseudomonas aeruginosa PA14 RSCV.

In other preferred embodiments, the activity of the compound used in the screening assay is dependent upon the presence of any one of the polynucleotides shown in FIG. 5B or 6A-6K, or a functional equivalent thereof. For example, the identified compound targets any one of the polynucleotides shown in FIG. 5B or 6A-6K or a functional equivalent thereof. In still other preferred embodiments, the expression of the regulator polynucleotide mediates phenotypic switching of the microbial cell in the presence of a high concentration of an antibiotic. In other preferred embodiments of the screening method, the polypeptide is expressed using an isolated polynucleotide that encodes a polypeptide that is substantially identical to any one of the polynucleotides shown FIGS. 5B and 6A-6K or a fragment thereof.

In another aspect, the invention features a screening method for identifying a compound that modulates an activity of a polypeptide that affects phenotype-mediated antibiotic-resistance in a microorganism. The method, in general, includes the steps of: (a) providing a microbial cell expressing a polypeptide that is substantially identical to any one of the polypeptides shown in FIGS. 5E and 6L-6V (or a polypeptide having at least 40% identity to any one of these sequences), wherein expression of the polypeptide, in the microbial cell, affects phenotype-mediated antibiotic-resistance in the microbial cell; (b) contacting the microbial cell with a compound; and (c) comparing an activity of the polypeptide in the presence of the compound with the activity in the absence of the compound; wherein a measurable difference in the activity indicates that the compound modulates the activity of the polypeptide that affects phenotype-mediated antibiotic-resistance in a microorganism. In preferred embodiments, the screening method identifies a compound that increases or decreases the activity of the polypeptide. Comparison of the activity of the polypeptide includes a variety of standard biochemical analyses including immunological assays.

In preferred embodiments, the microbial cell utilized in the screening assay is a phenotypic variant (e.g., Pseudomonas aeruginosa PA14 RSCV) having increased biofilm formation.

In other preferred embodiments, the regulator polypeptide is an isolated polypeptide that includes an amino acid sequence that is substantially identical to any one of the polypeptides shown in FIGS. 5E and 6L-6V (or a polypeptide having at least 40% identity to any one of these sequences). In particular, such a polypeptide has the ability to regulate phenotypic switching; to regulate biofilm-mediated antibiotic-resistance; to mediate phenotypic switching of the microbial cell in the presence of a high concentration of an antibiotic; or to affect susceptibility of the microbial cell to antibiotic treatment; or any combination thereof. In other preferred embodiments, the regulator polypeptide is an element of a two-component regulatory system. In yet other preferred embodiments, the polypeptide is expressed by an isolated polynucleotide that is substantially identical to any one of the nucleotide sequences shown in FIGS. 5B and 6A-6K (or a polynucleotide having at least 40% identity to any one of these sequences) or a fragment thereof, upon which the activity of the regulator polypeptide is increased or decreased.

Typically, the activity of the compound identified in the screening assay is dependent upon the presence of any one of the polypeptides shown in FIGS. 5E and 6L-6V or a functional equivalent thereof. In particular aspects of the screening assay, the compound targets or interacts with any one of the polypeptides shown in FIGS. 5E and 6L-6V or a functional equivalent thereof.

In another aspect, the invention features a screening method for identifying a compound that modulates microbial biofilm formation. This method, in general, includes the steps of: (a) culturing a microbial cell (e.g., Pseudomonas, Vibrio, Salmonella, or Staphylococcus) that includes a polypeptide that is substantially identical to any one of the polypeptides shown in FIGS. 5E and 6L-6V (or a polypeptide having at least 40% identity to any one of these sequences), wherein the microbial cell, upon culturing, forms a biofilm; (b) contacting the microbial cell with a compound; and (c) comparing microbial biofilm formation in the presence of the compound with microbial biofilm formation in the absence of the compound; wherein a measurable difference in the microbial biofilm formation indicates that the compound modulates biofilm formation.

In preferred embodiments, the screening method identifies a compound that increases or decreases biofilm formation. Typically, such biofilm formation is measured by using any standard method, for example, by assaying microbial aggregation (e.g., by using a microscope); using a salt aggregation test; or by using an attachment assay.

In preferred embodiments, the microbial cell is a phenotypic variant having increased biofilm formation when compared to its wild-type such as a small colony variant of Pseudomonas, Vibrio, Salmonella, or Staphylococcus. In other preferred embodiments, the small colony variant is a rough small colony variant of Pseudomonas, Vibrio, or Salmonella.

In yet other preferred embodiments, the activity of the compound utilized in the screening assay is dependent upon the presence of the polypeptide or a functional equivalent thereof. For example, the identified compound targets or interacts with the polypeptide or a functional equivalent thereof, resulting in increasing or decreasing its functional activity.

In still another embodiment, the expression of the polypeptide mediates phenotypic switching of the microbial cell in the presence of a high concentration of an antibiotic.

In another embodiment, the polypeptide is an isolated polypeptide that includes an amino acid sequence that is substantially identical to any one of the polypeptides shown in FIGS. 5E and 6L-6V (or a polypeptide having at least 40% identity to any one of these sequences), wherein expression of the polypeptide, in a microorganism, affects phenotype-mediated antibiotic-resistance in the microorganism.

In still another aspect, the invention features a method of treating a microbial infection involving a microorganism that forms a biofilm in a mammal. The method, in general, includes administering to the mammal a therapeutically-effective amount of a compound that induces or represses expression or activity of a polypeptide that includes a polypeptide that is substantially identical to any one of the polypeptides shown in FIGS. 5E and 6L-6V or a fragment thereof (or a polypeptide having at least 40% identity to any one of these sequences), wherein expression of the polypeptide or the fragment thereof, in a microorganism, affects phenotype-mediated antibiotic-resistance in the microorganism.

In another aspect, the invention features an isolated polypeptide including an amino acid sequence that is substantially identical to the amino acid sequence of any one of the polypeptides shown in FIG. 5F and FIGS. 7F-7J, each of which are encoded by a polynucleotide of the ORF3 region.

For example, with respect to the ORF3 region, the invention features an isolated polypeptide that includes an amino acid sequence that is at least 50% (and preferably 60%, 65%, 70%, 75%, 80%, 85%, 90%, or 95-99%) identical to the amino acid sequence of any one of the polypeptides shown in FIGS. 5F (SEQ ID NO:6) and 7F-7J (SEQ ID NOS:35-39), wherein expression of the polypeptide, in a microorganism, affects phenotype-mediated antibiotic-resistance in the microorganism. Preferably, the polypeptide includes the amino acid sequence shown in FIG. 7J (SEQ ID NO:39) or consists essentially of the amino acid sequence shown in FIGS. 5F (SEQ ID NO:6) and 7F-7I (SEQ ID NOS:35-38) or a fragment thereof.

In a related aspect, the invention features an isolated polypeptide fragment of an isolated polypeptide that includes an amino acid sequence having at least 50% identity to the amino acid sequence of the polypeptides shown in FIGS. 5F and 7F-7J. In preferred embodiments, such a polypeptide fragment includes at least 300 contiguous amino acid residues of the amino acid sequence shown in any one of FIGS. 5F and 7F-7J. In other embodiments, the fragment is at least 200 amino acid residues, or 100 amino acid residues of the polypeptides shown in FIGS. 5F and 7F-7J.

In another aspect the invention features an isolated polynucleotide molecule including a sequence substantially identical to any one of the polynucleotides shown in FIGS. 5C (SEQ ID NO:5) and 7A-7E (SEQ ID NOS:30-34). In preferred embodiments, the isolated polynucleotide molecule has at least 45%, 50%, 60%, 70%, 80%, 90%, or even 95% identity to any one of these molecules.

For example with respect to the ORF3 region, the invention features an isolated polynucleotide having at least 50% identity to any one of the nucleotide sequences shown in FIGS. 5C and 7A-7E, wherein expression of the polynucleotide, in a microorganism, affects phenotype-mediated antibiotic-resistance in the microorganism. In preferred embodiments, the isolated polynucleotide includes the nucleotide sequence shown in FIG. 5C or a complement thereof. In yet other preferred embodiments, the polynucleotide consists essentially of the nucleotide sequence shown in FIG. 5C or a fragment thereof.

In still other related aspects, the invention features a vector including any of the aforementioned isolated polynucleotides and a host cell that includes the vector.

The invention further features a variety of screening assays for identifying compounds that modulate phenotype-mediated antibiotic-resistance, biofilm formation, or biofilm-mediated antibiotic resistance. For example, the invention features a screening method that is useful for identifying a compound that modulates the gene expression of a regulator polynucleotide that affects phenotype-mediated antibiotic-resistance in a microorganism. Such a method includes the steps of: (a) providing a microbial cell (e.g., Pseudomonas, Vibrio, Salmonella, or Staphylococcus) that includes a polynucleotide substantially identical to the nucleotide sequences shown in FIGS. 5C and 7A-7E (or a polynucleotide having at least 45% identity to any one of these sequences), wherein expression of the polynucleotide, in the microbial cell, affects phenotype-mediated antibiotic-resistance in the microbial cell; (b) contacting the microbial cell with a compound; and (c) comparing the level of gene expression of the polynucleotide in the presence of the compound with the level of gene expression in the absence of the compound; wherein a measurable difference in gene expression indicates that the compound modulates gene expression of a regulator polynucleotide that affects phenotype-mediated antibiotic-resistance in a microorganism.

In preferred embodiments, the screening method identifies a compound that increases or decreases transcription of the regulator polynucleotide. In other embodiments, the screening method identifies a compound that increases or decreases translation of an mRNA transcribed from the regulator polynucleotide.

In other preferred embodiments, the microbial cell is a phenotypic variant (e.g., a small colony variant) having increased biofilm formation. Preferably, the small colony variant is a small colony variant of Pseudomonas, Vibrio, Salmonella, or Staphylococcus. In still other embodiments, the small colony variant is a rough small colony variant, for example, a rough small colony variant of Pseudomonas, Vibrio, Salmonella, or Staphylococcus. In a preferred embodiment, the rough small colony variant is Pseudomonas aeruginosa PA14 RSCV.

In other preferred embodiments, the activity of the compound used in the screening assay is dependent upon the presence of any one of the polynucleotides shown in FIGS. 5C and 7A-7E or a functional equivalent thereof. For example, the identified compound targets or interacts with any one of the polynucleotides shown in FIGS. 5C and 7A-7E or a functional equivalent thereof. In still other preferred embodiments, the expression of the regulator polynucleotide mediates phenotypic switching of the microbial cell in the presence of a high concentration of an antibiotic. In other preferred embodiments of the screening method, the polypeptide is expressed from an isolated polynucleotide that expresses a polypeptide that includes an amino acid sequence having at least 50% identity to any one of the amino acid sequences shown in FIGS. 5F and 7F-7J or a fragment thereof.

In another aspect, the invention features a screening method for identifying a compound that modulates an activity of a polypeptide that affects phenotype-mediated antibiotic-resistance in a microorganism. The method, in general, includes the steps of: (a) providing a microbial cell expressing a polypeptide that is substantially identical to any one of the polypeptides shown in FIGS. 5F and 7F-7J (or a polypeptide having at least 45% identity to any one of these sequences), wherein expression of the polypeptide, in the microbial cell, affects phenotype-mediated antibiotic-resistance in the microbial cell; (b) contacting the microbial cell with a compound; and (c) comparing an activity of the polypeptide in the presence of the compound with the activity in the absence of the compound; wherein a measurable difference in the activity indicates that the compound modulates the activity of the polypeptide that affects phenotype-mediated antibiotic-resistance in a microorganism. In preferred embodiments, the screening method identifies a compound that increases or decreases the activity of the polypeptide. Comparison of the activity of the polypeptide includes a variety of standard biochemical analyses including immunological assays.

In preferred embodiments, the microbial cell utilized in the screening assay is a phenotypic variant (e.g., Pseudomonas aeruginosa PA14 RSCV) having increased biofilm formation.

In other preferred embodiments, the regulator polypeptide is an isolated polypeptide that includes an amino acid sequence that is substantially identical to any one of the polypeptides shown in FIGS. 5F and 7F-7J (or a polypeptide having at least 45% identity to any one of these sequences). In particular, such a polypeptide has the ability to regulate phenotypic switching; to regulate biofilm-mediated antibiotic-resistance; to mediate phenotypic switching of the microbial cell in the presence of a high concentration of an antibiotic; or to affect susceptibility of the microbial cell to antibiotic treatment; or any combination thereof. In other preferred embodiments, the regulator polypeptide is an element of a two-component regulatory system. In yet other preferred embodiments, the polypeptide is expressed by an isolated polynucleotide substantially identical to any one of the nucleotide sequences shown in FIGS. 5C and 7A-7E (or by a polynucleotide having at least 45% identity to any one of these sequences) or a fragment thereof, upon which the activity of the regulator polypeptide is increased or decreased.

Typically, the activity of the compound identified in the screening assay is dependent upon the presence of any one of the polypeptides shown in FIGS. 5F and 7F-7J or a functional equivalent thereof. In particular aspects of the screening assay, the compound targets and interacts with the polypeptide of FIGS. 5F and 7F-7J or a functional equivalent thereof.

In another aspect, the invention features a screening method for identifying a compound that modulates microbial biofilm formation. This method, in general, includes the steps of: (a) culturing a microbial cell (e.g., Pseudomonas, Vibrio, Salmonella, or Staphylococcus) that includes a polypeptide substantially identical to any one of the amino acid sequences shown in FIGS. 5F and 7F-7J (or a polypeptide having at least 45% identity to any one of these sequences), wherein the microbial cell, upon culturing, forms a biofilm; (b) contacting the microbial cell with a compound; and (c) comparing microbial biofilm formation in the presence of the compound with microbial biofilm formation in the absence of the compound; wherein a measurable difference in the microbial biofilm formation indicates that the compound modulates biofilm formation.

In preferred embodiments, the screening method identifies a compound that increases or decreases biofilm formation. Typically, such biofilm formation is measured by using any standard method, for example, by assaying microbial aggregation (e.g., by using a microscope); using a salt aggregation test; or by using an attachment assay.

In preferred embodiments, the microbial cell is a phenotypic variant having increased biofilm formation when compared to its wild-type such as a small colony variant of Pseudomonas, Vibrio, Salmonella, or Staphylococcus. In other preferred embodiments, the small colony variant is a rough small colony variant of Pseudomonas, Vibrio, or Salmonella.

In yet other preferred embodiments, the activity of the compound utilized in the screening assay is dependent upon the presence of the polypeptide or a functional equivalent thereof. For example, the identified compound targets and interacts with the polypeptide or a functional equivalent thereof, resulting in increasing or decreasing its functional activity.

In still another embodiment, the expression of the polypeptide mediates phenotypic switching of the microbial cell in the presence of a high concentration of an antibiotic.

In another embodiment, the polypeptide is an isolated polypeptide that includes an amino acid sequence that is substantially identical to any one of the amino acid sequences shown in FIGS. 5F and 7F-7J (or a polypeptide having at least 45% identity to any one of these sequences), wherein expression of the polypeptide, in a microorganism, affects phenotype-mediated antibiotic-resistance in the microorganism.

In still another aspect, the invention features a method of treating a microbial infection involving a microorganism that forms a biofilm in a mammal. The method, in general, includes administering to the mammal a therapeutically-effective amount of a compound that induces or represses expression or activity of a polypeptide that includes an amino acid sequence that is substantially identical to any one of the amino acid sequences shown in FIGS. 5F and 7F-7J or a fragment thereof (or a polypeptide having at least 45% identity to any one of these sequences), wherein expression of the polypeptide or the fragment thereof, in a microorganism, affects phenotype-mediated antibiotic-resistance in the microorganism.

In preferred embodiments, the method further includes administering to the mammal a therapeutically-effective amount of an antibiotic. The treatment is particularly useful for treating patients having cystic fibrosis or a chronic infection or both. In other preferred embodiments, the microorganism treated using the method belongs to the genus Pseudomonas, Vibrio, Salmonella, or Staphylococcus.

In yet another aspect, the invention features a method of cleaning, disinfecting, or decontaminating a surface at least partially covered by a microorganism that forms a biofilm, the method involving contacting the microorganism with a cleaning composition including a compound that induces or represses expression or activity of a polypeptide that includes an amino acid sequence having at least 50% identity to the amino acid sequence of FIGS. 5E, 5F, 6L-6V, and 7F-7J or fragment thereof (or a polypeptide that is substantially identical to any one of these polypeptides), wherein expression of the polypeptide or the fragment thereof, in a microorganism, affects phenotype-mediated antibiotic-resistance in the microorganism.

The invention also features methods for identifying compounds useful for treating a patient having a biofilm infection. The method includes the steps of contacting a biofilm in vitro with (i) an antibiotic and (ii) a candidate compound (e.g., a compound that modulates the expression, at the transcriptional, post-transcriptional, translational, or post-translational levels, of a polynucleotide having at least 50% identity to any of the polynucleotides described herein (or that is substantially identical to a polynucleotide described herein), and determining whether the biofilm grows more slowly than (a) biofilm cells contacted with an antibiotic but not contacted with the test compound, and (b) biofilm cells contacted with the candidate compound but not with the antibiotic. In another embodiment, the biofilm is contacted with two or more different antibiotics. Exemplary antibiotics useful in the method include amikacin, aminoglicosides (e.g., tobramycin), aztreonam, carbenicillin, cephalosporines (e.g., ceftazidime or cefipime), chloramphenicol, gentamicin, levofloxacin, meropenem, piperacillin, tazobactam, tetracycline, and quinolones (e.g., ciprofloxacin). A candidate compound that reduces biofilm formation in the presence of an antibiotic (or combination of different antibiotics), but does not decrease biofilm formation in the absence of the antibiotic (or combination of different antibiotics), is a compound that is useful in combination therapy for treating a patient having a biofilm infection.

The invention further features a method for treating a patient having a biofilm infection, by administering to the patient an antibiofilm combination therapy that includes a compound identified as modulating expression, at the transcriptional, post-transcriptional, translational, or post-translational levels, of a polynucleotide having at least 50% identity to any of the polynucleotides described herein (or that is substantially identical to a polynucleotide described herein) and one or more antibiotics, including, but not limited to, amikacin, aminoglicosides (e.g., tobramycin), aztreonam, carbenicillin, cephalosporines (e.g., ceftazidime or cefipime), chloramphenicol, gentamicin, levofloxacin, meropenem, piperacillin, tazobactam, tetracycline, and quinolones (e.g., ciprofloxacin), simultaneously or within a period of time (e.g., 14 to 21 days) sufficient to inhibit the growth of the biofilm.

Preferably, the compound and antibiotic are administered within fifteen days of each other, more preferably within five or ten days of each other, and most preferably within twenty-four hours of each other or even simultaneously. Exemplary biofilms treated according to any of the methods described herein are those formed by bacteria, including but not limited to, Pseudomonas, Staphylococcus, Salmonella, Vibrio, Haemophilus, Mycobacterium, Helicobacter, Burkholderia, or Streptococci.

In a related aspect, the invention also features a method for treating a patient having a biofilm such as one formed from Pseudomonas (e.g., Pseudomonas aeruginosa). In this method, a patient is administered (a) a first compound (e.g., a compound that modulates the expression, at the transcriptional, post-transcriptional, translational, or post-translational; of a polynucleotide having at least 50% identity to a polynucleotide described herein (or that is substantially identical to a polynucleotide described herein)), and (b) one or more antibiotics (such as amikacin, aminoglicosides (e.g., tobramycin), aztreonam, carbenicillin, cephalosporines (e.g., ceftazidime or cefipime), chloramphenicol, gentamicin, levofloxacin, meropenem, piperacillin, tazobactam, tetracycline, and quinolones (e.g., ciprofloxacin). If desired, the therapy includes administration of two antibiotics according to standard methods known in the art. Such dual antibiotic combinations most preferably include high-dose tobramycin plus meropenem, meropenem plus ciprofloxacin, or tobramycin (4 μg/ml), or cefipime. Other preferred combinations include piperacillin plus tazobactam, or piperacillin plus ciprofloxacin. The antibiotic and compound combination therapy are preferably administered simultaneously or within a period of time sufficient to inhibit the growth of the biofilm.

In any of the foregoing treatments, the compound and antibiotic included in the combination therapy are preferably administered to the patient as part of a pharmaceutical composition that also includes a pharmaceutically acceptable carrier. Preferred modes of administration include intramuscular, intravenous, inhalation, and oral administration, or a combination thereof.

The antibiofilm combinations of the invention can also be part of a pharmaceutical kit. Preferably, the first compound (e.g., a compound identified as modulating expression, at the transcriptional, post-transcriptional, translational, or post-translational levels, of a polynucleotide or polypeptide having at least 50% identity to any one of the polynucleotide or polypeptide sequences described herein (or that is substantially identical to any one of the polynucleotides or polypeptides described herein)) and the second compound, an antibiotic, are formulated together or separately and in individual dosage amounts.

Combination therapy may be provided wherever antibiotic treatment is performed: at home, the doctor's office, a clinic, a hospital's outpatient department, or a hospital. Treatment generally begins at a hospital so that the doctor can observe the therapy's effects closely and make any adjustments that are needed. The duration of the combination therapy depends on the kind of biofilm being treated, the age and condition of the patient, the stage and type of the patient's biofilm infection, and how the patient's body responds to the treatment. Drug administration may be performed at different intervals (e.g., daily, weekly, or monthly) and the administration of each agent can be determined individually. Combination therapy may be given in on-and-off cycles that include rest periods so that the patient's body has a chance to build healthy new cells and regain its strength.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “polypeptide” is meant any chain of amino acids, regardless of length or post-translational modification (for example, glycosylation or phosphorylation).

By an “isolated polypeptide” is meant a polypeptide of the invention that has been separated from components which naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source (for example, a pathogen); by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule (e.g., a polynucleotide) exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80%, and most preferably 90% or even 95% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a polypeptide of the invention.

By “positioned for expression” is meant that the polynucleotide of the invention (e.g., a DNA molecule) is positioned adjacent to a DNA sequence which directs transcription and translation of the sequence (i.e., facilitates the production of, for example, a recombinant polypeptide of the invention, or an RNA molecule).

By “purified antibody” is meant an antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody which recognizes and binds a polypeptide of the invention but which does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “inhibiting biofilm formation” is meant the ability of a candidate compound to decrease the development or progression of biofilm formation. Preferably, such inhibition decreases biofilm formation by at least 1% to 5%, more preferably by at least 10%, 15%, 20%, or 25%, and most preferably by at least 30% to 50%, as compared to biofilm formation in the absence of the candidate compound in any appropriate pathogenicity assay (for example, those assays described herein). In one particular example, inhibition is measured by continuous culture conditions of a microbe exposed to a candidate compound or extract, a decrease in the level of biofilm formation relative to the level of biofilm formation of the microbe not exposed to the compound indicating compound-mediated inhibition of biofilm formation.

By “biofilm regulator polynucleotide” is meant a polynucleotide encoding a cellular component (e.g., PvrR) that modulates phenotypic switching, such as a phenotypic switch that occurs during biofilm formation, disintegration, or both.

By “phenotypic switching” is meant the reversible alteration of one or more phenotypic characteristics. Such an alteration typically occurs, for example, when a wild-type microbe develops into an antibiotic-resistant phenotypic variant or when an antibiotic-resistant phenotypic variant develops into a wild-type microbe.

By “immunological assay” is meant an assay that relies on an immunological reaction, for example, antibody binding to an antigen. Examples of immunological assays include ELISAs, Western blots, immunoprecipitations, and other assays known to the skilled artisan.

By a “two-component regulatory system” is meant a regulatory system that includes at least two components such as a sensor that senses an environmental signal and a response regulator that modulates one or more effectors.

By “aggregation” is meant a collection of two or more individual microorganisms into a mass or clump, such that the individuals form an aggregated microbial unit. Aggregation can be measured using assays provided herein. Examplary assays include visual inspection, measuring attachment to a surface, or by assaying for biofilm formation using methods known to the skilled artisan.

By “pathogenicity” is meant the ability of a microorganism to cause disease. A microorganism that forms a biofilm, has increased antibiotic resistance, or displays phenotypic variation is more pathogenic than a wild-type microorganism in that it is less susceptibile to conventional antibiotic treatment.

The invention provides a number of targets that are useful for the development of drugs that specifically block the biofilm formation of a microbe. In addition, the methods of the invention provide a facile means to identify compounds that are safe for use in eukaryotic host organisms (i.e., compounds which do not adversely affect the normal development and physiology of the organism), and efficacious against pathogenic microbes (i.e., by suppressing the virulence of a pathogen). In addition, the methods of the invention provide a route for analyzing virtually any number of compounds for an anti-virulence effect with high-volume throughput, high sensitivity, and low complexity. The methods are also relatively inexpensive to perform and enable the analysis of small quantities of active substances found in either purified or crude extract form.

Other features and advantages of the invention will be apparent from the detailed description, and from the claims.

### BRIEF DESCRIPTION OF THE DRAWINGS

FIG. 1A shows the reversion of PA14 rough small colony variants (RSCV) to the wild-type phenotype as observed at the edges of the colonies (arrow) after 2-3 days incubation on antibiotic free LB agar at room temperature.

FIG. 1B shows a confocal scanning laser microscopic analysis of bacterial aggregates (arrows) formed by wild-type PA14 and PA14 RSCV expressing green fluorescent protein (GFP) after overnight growth in liquid broth. Scale bar, 25 μm.

FIG. 1C shows the attachment of wild-type PA14 and antibiotic resistant variants to polyvinylchloride plastic (PVC) after 6 hours of growth.

FIG. 1D shows a confocal laser scanning microscope analysis of biofilm formed by wild-type PA14 and PA14 RSCV expressing GFP in flow-chambers under continuous culture conditions. Scale bar, 50 μm.

FIG. 1E shows PA14 and PA14 RSCV biofilm resistance to tobramycin as determined by measuring viable biomass on 45 hour-old established biofilms before (filled bars) and after (open bars) 36-hour tobramycin (200 μg/ml) treatment.

FIG. 2A shows the effect of different environmental stimuli on the rate of appearance of antibiotic resistant variants. This was determined by growing the cultures of wild-type PA14 under the specified conditions on media containing 200 μg/ml kanamycin.

FIG. 2B shows the minimal inhibitory concentrations of kanamycin for strain PA14 using the different conditions specified.

FIG. 3A shows the reversion of PA14 RSCV present in sputum samples of a cystic fibrosis patient (designated “CF 5”) as observed on the edges of the variant colonies (arrow) after prolonged incubation on antibiotic-free medium at room temperature.

FIG. 3B shows the increased attachment to PVC plastic of antibiotic resistant variants SCV 42 and SCV 43 obtained after plating CF isolates CF 42 and CF 43 on tobramycin (10 μg/ml).

FIG. 4A shows the attachment to PVC plastic of PA14, antibiotic resistant variants, and PA14 RSCV carrying pEd202 (PA14 RSCV/pED202) or pUCP19 (PA14 RSCV/pUCP19) after 4 hours of growth was quantitated.

FIG. 4B shows the predicted amino acid sequence alignment of PvrR with the sequences that correspond to VieA from V. Cholerae and the P. aeruginosa PAO1 putative response regulator PA3947 (PAO1 RR). Numbers above the scale indicate number of amino acids. Lower panel contains domain family numbers according to ProDom nomenclature.

FIG. 4C shows that the pvrR gene is flanked by two open reading frame regions (ORFs), designated ORF1 and ORF3, with the same transcriptional orientation. Start codons within ORFs were assigned based on visual inspection for appropriately spaced ribosome-binding sequences.

FIG. 4D shows the number of variants resistant to kanamycin (200 μg/ml). This was evaluated after plating overnight cultures of PA14 and PA14 overexpressing PvrR (PA14/pED202).

FIG. 4E shows the attachment to PVC plastic of PA14 and PA14 overexpressing PvrR (PA14/pED202) after 12 hours of growth, quantitated as described herein.

FIG. 4F shows the number of antibiotic resistant variants for PA14 and the pvrR mutant (ΔpvrR) as determined by plating overnight cultures on LB agar containing kanamycin (200 μg/ml).

FIG. 5A shows the nucleic acid sequence of pvrR (SEQ ID NO:1).

FIG. 5B shows the nucleic acid sequence of an ORF1 polynucleotide (SEQ ID NO:3). This polynucleotide sequence begins at nucleotide 1504 and ends at nucleotide 2919 of SEQ ID NO: 7 as shown in FIG. 5G.

FIG. 5C shows the nucleic acid sequence of an ORF3 polynucleotide (SEQ ID NO:5). This polynucleotide sequence begins at nucleotide 4385 and ends at nucleotide 6379 of SEQ ID NO:7 as shown in FIG. 5G.

FIG. 5D shows the deduced amino acid sequence of PvrR (SEQ ID NO:2).

FIG. 5E shows the deduced amino acid sequence of a polypeptide (SEQ ID NO:4) encoded by the polynucleotide shown in FIG. 5B.

FIG. 5F shows the deduced amino acid sequence of a polypeptide (SEQ ID NO:6) encoded by the polynucleotide shown in FIG. 5C.

FIG. 5G shows the nucleic acid sequence (SEQ ID NO:7) that includes the pvrR gene (SEQ ID NO:1), and the ORF1 (SEQ ID NOS:3 and 8-18) and ORF3 (SEQ ID NOS:5 and 30-34) regions. The start and stop codons for the identified open reading frames are highlighted.

FIGS. 6A-6K show the nucleotide sequences of several open reading frames identified in the ORF1 region (SEQ ID NO:8 begins at nucleotide 124 and ends at nucleotide 2919; SEQ ID NO:9 begins at nucleotide 199 and ends at nucleotide 2919; SEQ ID NO:10 begins at nucleotide 217 and ends at nucleotide 2919; SEQ ID NO:11 begins at nucleotide 256 and ends at nucleotide 2919; SEQ ID NO:12 begins at nucleotide 295 and ends at nucleotide 2919; SEQ ID NO:13 begins at nucleotide 307 and ends at nucleotide 2919; SEQ ID NO:14 begins at nucleotide 511 and ends at nucleotide 2919; SEQ ID NO:15 begins at nucleotide 760 and ends at nucleotide 2919; SEQ ID NO:16 begins at nucleotide 790 and ends at nucleotide 2919; SEQ ID NO:17 begins at nucleotide 919 and ends at nucleotide 2919; and SEQ ID NO18 begins at nucleotide 1429 and ends at nucleotide 2919).

FIGS. 6L-6V show the deduced amino acid sequences of the polypeptides (SEQ ID NOS:19-29) identified in FIGS. 6A-6K above.

FIGS. 7A-7E show the nucleotide sequence of several open reading frames identified in the ORF3 region (SEQ ID NO:30 begins at nucleotide 4388 and ends at nucleotide 6379; SEQ ID NO:31 begins at nucleotide 4550 and ends at nucleotide 6379; SEQ ID NO:32 begins at nucleotide 4572 and ends at nucleotide 6379; SEQ ID NO:33 begins at nucleotide 4880 and ends at nucleotide 6379; and SEQ ID NO:34 begins at nucleotide 5258 and ends at nucleotide 6379).

FIGS. 7F-7J show the deduced amino acid sequences of the polypeptides (SEQ ID NOS:35-39) identified in FIGS. 7A-7E above.

### DETAILED DESCRIPTION

Overview

Pseudomonas aeruginosa is the most important pathogen in the lungs of cystic fibrosis (CF) patients. Colonization of the CF lung by P. aeruginosa persists despite the use of long-term antibiotic therapy, since antibiotic treatment rarely results in eradication of the infection. Reports have suggested a direct link between resistance to antimicrobial compounds and the ability of P. aeruginosa to form biofilm in CF lungs. Other hypotheses explain P. aeruginosa antibiotic resistance by postulating that factors within the CF respiratory tract select for phenotypic variants suited to survive antimicrobial treatment. As is discussed below, we have determined that a clinical isolate of P. aeruginosa, strain PA14, was capable of growing under inhibitory concentrations of the antibiotic kanamycin (up to 40 times the susceptibility level of the strain) when bacteria had undergone phenotypic variation. The antibiotic resistant variant colonies obtained from kanamycin plates were smaller in size and had a different colony morphology compared to the wild-type. Analysis of the phenotype of PA14 RSCV indicated that these variants exhibited increased aggregation and attachment to glass tubes and polyvinylchloride plastic (PVC) as a result of enhanced surface hydrophobicity. Consistent with these observations, several PA14 RSCV clones were hyperpiliated when analysed by transmission electron microscopy. Moreover, examination of biofilms cultivated in flow chamber cells showed that PA14 RSCV formed more biofilm and faster than the wild-type strain. The biofilm formed by PA14 RSCV also showed increased resistance to tobramycin relative to wild-type PA14 biofilm. Similar results were obtained for several CF isolates using different antibiotics (including tobramycin), suggesting that nonspecific antibiotic resistance acquired through phenotypic variation is a common mechanism in P. aeruginosa. Moreover, analysis of sputum samples taken from CF patients revealed that antibiotic treatment selects for antibiotic resistant variants. The frequency with which antibiotic resistant variants appeared was also affected by environmental stimuli. Environmental stimuli such as salt concentration, temperature, and bacterial media altered the frequency of appearance of resistant variants.

To identify components involved in the regulation of antibiotic resistance mediated by phenotypic variation, a library of PA14 chromosomal DNA was transferred into PA14 RSCV and screened for colonies displaying wild-type colony size and morphology. This led to the identification of a clone, pED202, that restored the colony, the autoagglutination, and attachment phenotypes of PA14 RSCV variants to wild-type. pED202 contained a single gene (designated pvrR for phenotype variant regulator) that showed sequence similarities to response regulator elements of the two-component regulatory system found in Vibrio cholerae response regulator VieA, and in P. aeruginosa strain PAO1 (ORF PA3947).

Consistent with the putative role of PvrR in the regulation of phenotypic switching, overexpression of PvrR from pED202 in wild-type PA14 resulted in reduced attachment to PVC plastic. Moreover, examination of the frequency of resistant variants obtained from kanamycin plates showed a reduction in the number of colonies resistant to antibiotic obtained from the PvrR overexpressing strain. An in-frame deletion of pvrR (ΔpvrR) constructed in PA14 increased frequency of appearance of resistant variants on kanamycin plates with respect to the wild-type, confirming the involvement of pvrR in the regulation of phenotypic switching. These results suggested that PvrR might be acting upstream of the switch, since inactivation of pvrR by mutation did not result in conversion to the variant type.

Below we describe the cloning and characterization of PvrR, a regulator of biofilm-mediated antibiotic resistance and a target for compounds useful in antibacterial therapy, along with antibiotics, for the treatment of chronic infections and biofilm control in medical and industrial settings. In addition, we describe the identification of open reading frame regions, designated ORF1 and ORF3, that flank the pvrR gene. The following examples are for the purposes of illustrating the invention, and should not be construed as limiting.

Appearance of Rough Small Colony Variants with Increased Antibiotic Resistance

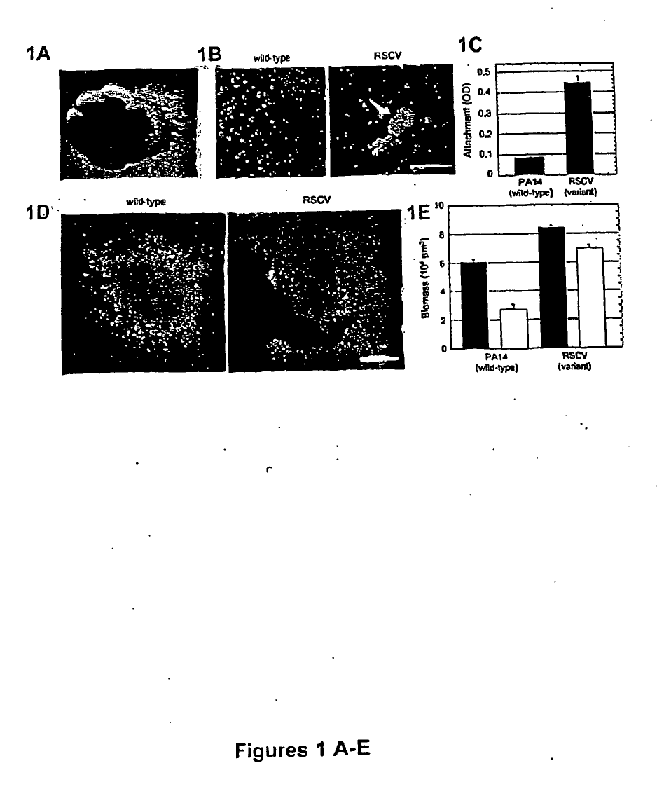

When cultured under high concentrations of antibiotic, Pseudomonas aeruginosa PA14 was found to shift its development to a rough small colony phenotype, leading to the production of antibiotic resistant colonies. To induce such phenotypic variants, an overnight culture of P. aeruginosa strain PA14 (UCBPP-PA14) was inoculated onto Luria-Bertani (LB) containing 200 μg/ml of kanamycin, incubated at 37° C. for 48 hours, at which time, antibiotic resistant rough small variants were isolated. Antibiotic resistant colonies arose at a frequency of 10⁻⁶-10⁻⁷. The colonies identified on these plates were one-tenth the size of wild type and exhibited a rough phenotype compared to the smooth colony type of wild-type PA14. One class of kanamycin resistant variants (approximately 30%) exhibited a rough phenotype compared to the smooth colony type of wild-type PA14. When incubated for three to five days in LB media without antibiotic at room temperature, the rough phenotype reverted to the wild-type phenotype (FIG. 1A), indicating that the phenotypic changes were transient, and not due to mutation. In addition to being resistant to kanamycin, (up to 40 times the susceptibility level of the wild-type), 8 individual PA14 RSCV colonies tested were also resistant to amikacin (30 μg/ml), carbenicillin (300 μg/ml), gentamicin (30 μg/ml), tobramycin (10 μg/ml), and tetracycline (150 μg/ml). Consistent with this latter result, antibiotic resistant variants were also obtained at frequencies of about 10⁻⁷ by plating overnight cultures of PA14 on media containing similar concentrations of the antibiotics mentioned above. Although RSCV colonies were smaller than wild-type, their small colony size was not a consequence of slow growth since the generation time of RSCV in liquid medium was not significantly different from that of the wild-type, even in LB containing 200 μg/ml kanamycin.

Phenotypic Changes Associated with Appearance of Resistance

To establish a connection between the phenotypic switch from wild-type to small variant colony and the emergence of antibiotic resistance, comparative attachment, agglutination, and biofilm formation studies of wild-type PA14 and PA14 RSCV were conducted.

The results of these experiments showed that PA14 RSCV formed visible bacterial aggregates when overnight liquid cultures were left without shaking at room temperature (FIG. 1B). Moreover, abundant bacterial aggregates formed when liquid cultures were grown with gentle agitation, indicating that PA14 RSCV had increased cell-cell attachment compared to the wild-type phenotype.

In addition to the autoagglutination phenotype, PA14 RSCV developed a visible biofilm on the walls of glass tubes after overnight incubation in liquid culture. Wild-type PA14 failed to form a similar biofilm under these conditions. These results indicated that cell-surface interactions, as well as cell-cell interactions were increased in the variant. Consistent with this observation, PA14 RSCV were found to have increased attachment to PVC plastic (FIG. 1C) in assays conducted in 96-well microtiter plates. When reversion was induced in PA14 RSCV, the reverted bacteria showed wild-type levels of both agglutination and attachment to glass and PVC plastic.

To quantitatively assess differences between the strains, standard bacterial attachment assays were performed in 96-well polyvinylchloride (PVC) plastic plates according to the methods described by O'Toole et al. (Mol. Microbiol. 30: 295, 1998). Overnight cultures of PA 14 and PA 14 RSCV were diluted to an OD₆₀₀ of 0.1 in fresh minimal M63 salts supplemented with glucose (0.3%), MgSO₄ (1 mM), and casamino acids (CAA, 0.5%). Aliquots of 100 μl were next dispensed into the wells of PVC plastic microtiter plates and incubated for 6 hours at 37° C. The attachment of bacteria to the walls of the microtiter well was then detected by staining with 1% crystal violet dissolved in water. Dye not associated with bacteria was removed by thorough rinsing with water. Bacteria-associated dye was solubilized using 95% ethanol and absorbance was determined at OD₅₅₀.

In addition, since the ability of bacteria to attach to each other and to surfaces depends in part on the interaction of hydrophobic domains (Drumm et al., J. Clin. Invest. 84:1588, 1989), the hydrophobic surface properties of the wild-type and PA14 RSCV were determined using a standard salt aggregation test (Sherman et al., Infect. Immun. 49:797, 1985). 5×10⁸ bacteria per ml in 0.025 ml were mixed on a microscope slide with an equal volume of ammonium sulfate in 0.002 M sodium phosphate, pH 6.8. The ammonium sulfate concentrations varied from 0.0625 M to 4.0 M, and the presence of salt-induced bacterial aggregation was monitored for 2 minutes at room temperature by phase-contrast microscopy. Agglutination in salt concentrations of less than 0.1 M is taken as an indication of the presence of a hydrophobic bacterial surface. Hydrophilic surfaces were demonstrated by the agglutination of bacteria only in high salt concentrations (2.0 to 4.0 M).

The data obtained from the salt aggregation tests showed that PA14 RSCV were agglutinated at a lower salt concentration (0.125 M) compared to the wild-type PA14 (0.5 M), suggesting that PA14 RSCV has a higher degree of surface hydrophobicity than the wild-type. Therefore, the data indicated that a change in the hydrophobic properties of the surface of the bacteria was partially responsible for the general increase in surface attachment of the PA14 RSCV phenotypic variant. To further demonstrate the role of hydrophobicity in surface attachment, PA14 RSCV were cultured in the presence of tetramethyl urea (TMU), a hydrophobic bond-breaking agent, at a concentration of 200 mM. Addition of TMU to the culture media was found to reduce the attachment of the phenotypic variant PA14 RSCV to wild-type levels, confirming the hydrophobic nature of the bacterial surface. TMU, at the concentration used in these assays, did not affect cell viability.

Transmission electron microscopic analysis of several PA14 RSCV clones revealed that they were hyperpiliated, which is consistent with the increased hydrophobicity and agglutination phenotypes. However, the various phenotypes of PA14 RSCV were not simply a consequence of hyperpiliation since a hyperpiliated mutant of P. aeruginosa PA14, pilU, exhibited only marginally enhanced hydrophobicity and attachment to PVC plastic and did not exhibit enhanced resistance to antibiotics (data not shown). These results are consistent with previous reports which indicated that phenotypic variation in Gram-negative bacteria involve changes in expression of a number of surface structures, outer membrane proteins, and lipopolysaccharides resulting in altered aggregation and colony morphology. Several PA14 RSCV clones were tested in the experiments described above and all exhibited similar phenotypes. A single PA14 RSCV clone was therefore chosen for further analysis.

To determine whether the antibiotic resistant phenotype of PA14 RSCV is associated with altered biofilm formation, PA14 RSCV was cultured under biofilm-forming conditions as follows. For biofilm characterization, PA14 RSCV biofilms were cultivated under continuous culture conditions in flow-chambers with channel dimensions of 12 by 52 by 2 mm. Flow media consisted of M63 supplemented with 0.5% casamino acids and 0.3% glucose. For measurement of biofilm resistance, bacteria were cultivated in flow-chambers with channel dimensions of 1 by 40 by 4 mm (Stovall Inc., Greensboro, N.C.). In this case, flow media consisted of FAB medium (0.1 mM CaCl₂, 0.01 mM Fe-EDTA, 0.15 mM NH₄SO₄, 0.33 mM Na₂HPO₄, 0.2 mM KH₂PO₄ and 1 mM MgCl₂) supplemented with casamino acids (0.5%) and sodium citrate (10 mM). Flow-cells in both cases were inoculated with 100-fold dilutions of overnight cultures of PA14 and PA14 RSCV carrying the green fluorescent protein (GFP) in plasmid SMC21, a derivative of pSMC2 (Bloemberg et al., Appl. Environ. Microbiol. 63: 4543-4551, 1997). After inoculation, the medium flow was stopped for 1 hour. Medium flow was then resumed at a rate of 0.2 ml/min using a peristaltic pump (IsmaTec, Zurich, Switzerland), and the flow-cell system was incubated at 37° C. Analysis of biofilm spatial structures was performed using confocal scanning laser microscopy (CSLM) using a Leica TCS SP system (Leica Lasertechnik, GmgH, Heidelberg, Germany). Image analysis of antibiotic-treated biofilms was done in structures contained within serial section stacks of images delimited by freehand drawing. Pixel intensities unique to GFP-labeled bacteria and surrounding biofilm were established by the threshold limit technique. The volume (in μm³) of individual biofilm structures was determined from serial sections using ImageSpace software (Molecular Dynamics, Sunnyvale, Calif.).

The results from these studies showed that the PA14 RSCV phenotypic variant formed not only more biofilm than the wild-type strain, but also formed biofilm faster (RSCV microcolonies appeared 4-5 hours earlier than wild-type). Moreover, PA14 RSCV and wild-type PA14 displayed significantly different patterns of biofilm development. Wild-type PA14 initially formed regularly-spaced, flat, circular, microcolonies that eventually developed into ball-shaped microcolonies. In contrast, PA14 RSCV formed irregularly shaped three-dimensional structures that were densely packed with bacteria, without the typical microcolony morphology (FIG. 1D). Finally, the biofilm structures formed by PA14 RSCV were larger in size than the wild-type microcolonies, and biofilms from PA14 RSCV contained more biomass than the wild-type.

To determine whether PA14 and PA14 RSCV biofilms exhibited antibiotic resistance that paralleled the resistance observed on plates containing antibiotic, established PA14 and PA14 RSCV biofilms grown in flow chambers were exposed to a continuous flow of tobramycin (200 μg/ml). Viable biomass was measured by CSLM analysis of GFP-tagged PA14 and PA14 RSCV cells using GFP expression as a viability marker as described previously (FIG. 1E). Consistent with the results obtained in plates, the biofilm formed by PA14 RSCV was more resistant to tobramycin treatment than the wild-type PA14 biofilm.

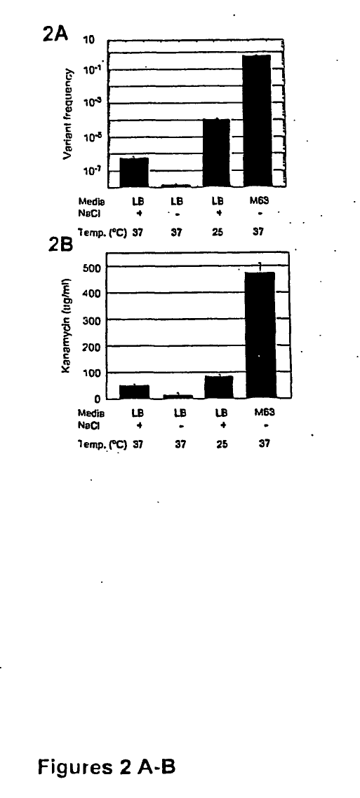

Phenotypic variation is a common phenomenon in Gram-negative bacteria that often involves environmentally regulated changes in observable phenotypes produced by modifications in surface components. The effect that different environmental stimuli had on the appearance of kanamycin-resistant phenotypic variants was examined. Bacteria were grown in LB broth, or in supplemented LB with appropriate antibiotics at the indicated temperature with aeration. As shown in FIG. 2A, a 40-fold increase in the frequency of appearance of resistant variants (not just PA14 RSCV) was observed on LB media supplemented with 85 mM NaCl as compared to the same medium without NaCl. Moreover, the frequency of variants increased 200-fold when plates were incubated at 25° C. compared to 37° C. (FIG. 2A). Finally, a dramatic 10⁶-fold increase was obtained on minimal M63 salts as compared to LB medium (FIG. 2A). Minimal salt media consisted of M63 supplemented with 0.3% glucose, 1 mM MgSO₄, and 0.5% casamino acids. Importantly, there was a correlation between the frequency of appearance of kanamycin resistant variants on plates and minimal inhibitory concentrations (MICs) of kanamycin in liquid culture for the wild-type PA14 using the culture conditions described above (FIG. 2B). For example, the high frequency of resistant variants obtained on M63 correlated with the relatively high concentration of kanamycin (475 μg/ml) required to inhibit the growth of PA14 in M63 liquid medium (FIGS. 2A and 2B). These data indicated that the components involved in the formation of antibiotic resistant variants are differentially regulated by environmental signals. Moreover, the data indicated that the portion of the population that becomes resistant to antibiotics through phenotypic variation was largely dependent on environmental conditions.

Small Colony Variants in CF Sputum Samples

The presence of phenotypic variants with small colony phenotypes has been reported in cystic fibrosis (CF) patients (Haussler et al., Clin. Infect. Dis. 29:621, 1999). Emergence of this and other variant phenotypes in the CF lung has also been linked to prolonged antibiotic treatment (McNamara et al., Int. J. Antimicrob. Agents 14:117, 2000; Kahl et al., J. Infect. Dis. 177:1023, 1998). To investigate whether antibiotic treatment in P. aeruginosa CF infections results in selection for resistant variants, we looked for the presence of small colony variants in CF sputum samples.

Five CF sputum samples from the Clinical Microbiology Laboratory at Massachusetts General Hospital were suspended in 5 ml of 10 mM MgSO₄. Serial dilutions of the samples were then plated onto cetrimide agar plates with and without antibiotics. The plates were screened for the presence of P. aeruginosa after 24 and 48 hours of incubation at 37° C. The identity of the colonies was later confirmed by probing colony lifts with the exotoxin A gene from P. aeruginosa. To this end, the EcoRI-HindIII fragment of plasmid pRGI containing the exoA gene (Samadpour et al., J. Clin. Microbiol. 26:2319-23, 1988) was gel isolated and labeled using a random priming kit (Boehringer, Mannheim, Indianapolis, Ind.). Colonies were transferred to nylon membranes and hybridizations were performed according to the manufacturer's recommendations (NEN Research Products, Boston, Mass.). Identification of colonies carrying the exoA gene was then performed using a Phosphorimager (Amersham Pharmacia Biotech Inc., Piscataway, N.J.).

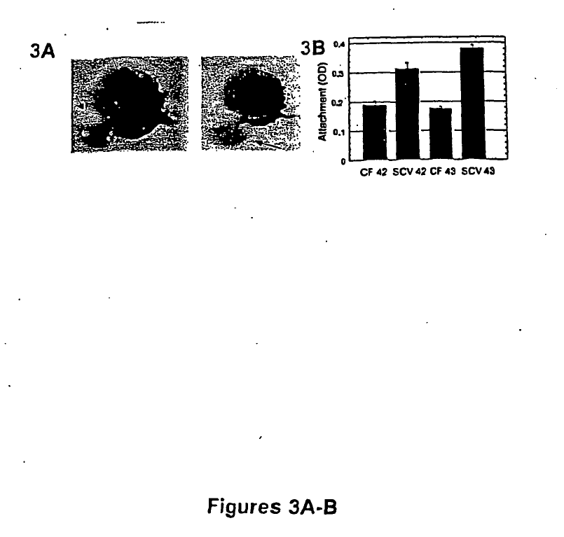

Five sputum samples obtained from five CF patients were evaluated for the presence of small colony variant bacteria. Two out of five sputum samples obtained from CF patients (patients 5 and 38) contained 100% rough small colony variants (Table 1) that reverted to a wild-type colony morphology upon prolonged incubation on antibiotic-free medium (FIG. 3A). Importantly, both samples 5 and 38 corresponded to patients that were undergoing antibiotic treatment at the time the samples were obtained (intravenous (IV) amikacin/ceftazidime for two days and oral (O) levofloxacin/inhaled (I) tobramycin for six weeks respectively Table 1).

TABLE 1

| | Sample 5 | Sample 38 | Sample 41 | Sample 42 | Sample 43 |
|---|---|---|---|---|---|
| Antibiotic treatment of CF | Amikacin(IV) | Tobramycin (I) | none | none | none |
| patients | Ceftazidime(IV) | Levofloxacin(0) | | | |
| Small Colony variants in | 100 | 100 | <0.11 | 0.00 | <0.12 |
| sputum sample (%) | | | | | |
| Variants resistant to amikacin | 100 | 100 | 15 | 5 | 0.2 |
| (%) | | | | | |
| Variants resistant to gentamicin | 100 | 100 | 10 | 6.6 | 0.5 |
| (%) | | | | | |
| Variants resistant to | 30 | 32 | 0 | 0 | Not done |
| tetracycline (%) | | | | | |
| Variants resistant to | 50 | 100 | 0.10 | 0 | 0.5 |
| tobramycin (%) | | | | | |

Table 1 shows the presence of small colony P. aeruginosa variants in sputum samples from five CF patients. The presence of P. aeruginosa antibiotic resistant small colony variants was determined by plating CF sputum samples on cetrimide agar with and without the indicated antibiotics.

Moreover, there was 29% enrichment in small colony variants in samples taken on two consecutive days from the patient that was undergoing intravenous antibiotic treatment.

As shown in Table 1, 30-100% of the small colony variants present in samples 5 and 38 were resistant to four different antibiotics (amikacin, gentamicin, tetracycline, and tobramycin) at concentrations equal to or higher than the minimal bactericidal concentration (MBC) of their respective reverted colonies. The proportion of small colony variants present in the samples that showed resistance to amikacin, gentamicin, tetracycline, and tobramycin was analyzed by simultaneously plating the sputum samples in cetrimide agar with and without antibiotics. The data obtained were compared to MBCs of the reverted colonies for the antibiotics in which variants were obtained In vitro susceptibility (MBC) to the different antibiotics used during the assays was determined by a standard tube dilution procedure described by Bailey and Scott (Diagnostic Microbiology, 313-329, 1974).

Although the other three CF sputum samples (41, 42 and 43) appeared to contain either a small proportion or no detectable small colony variants when plated on antibiotic free media, they did contain a considerable number (0.5-15%) of antibiotic resistant variants (Table 1). This discrepancy was due to the fact that it took the small colony variants 36-40 hours to form visible colonies, at which time the fast growing wild-type bacteria present in the sputum samples had overgrown the antibiotic free plates. Resistant variants with small colony phenotypes obtained from plating CF isolates 42 and 43 on media containing tobramycin (a front-line antibiotic used for the treatment of P. aeruginosa infections) exhibited increased attachment to PVC plastic (FIG. 3B).

Identification of the Phenotypic Variation Regulator Gene

Phenotypic variation is a common mechanism in Gram-negative bacteria, and involves changes in observable phenotypes produced by modifications in surface components such as fimbriae, flagella, outer membrane proteins, and lipopolysaccharides. In the mushroom pathogen P. tolaasii, Greewal et al. (J. Bacteriol. 177:4658, 1995) identified a two-component regulatory element responsible for the phenotypic switch from smooth to rough phenotype that involved changes in colony morphology and motility. Since the phenotype displayed by PA14 RSCV was transient and involved alterations in surface properties, we hypothesized that a regulatory component was also responsible for the phenotypic switch observed in PA14.

To identify this component, a genomic library of strain PA14 constructed in the cosmid vector pJSR1 (Rahme et al., Science 268:1899, 1995) was mobilized in masse into PA14 RSCV by triparental mating using helper strain pRK2013 (Figurski et al., Proc. Natl. Acad. Sci. USA 76:1648, 1979). The resulting transconjugants were screened visually for colonies showing wild-type size and morphology (smooth colony phenotype). Individual transconjugants that showed wild-type characteristics were used to isolate the corresponding cosmids which were then reintroduced into PA14 RSCV to confirm the reversion of the phenotype. Moreover, cosmid DNA from the transconjugants was digested to completion with the restriction enzymes EcoRI, PstI, and HindIII and separated by electrophoresis on a 0.7% agarose gel.

A total of 2,500 transconjugants were screened for colonies displaying wild-type PA14 colony size and morphology. Two transconjugants that showed wild-type phenotypes were isolated, indicating that the inserts contained in the cosmids were able to induce reversion from small colony variant to wild-type phenotype. Two cosmid clones were isolated and reintroduced in PA14 RSCV to test for restoration of wild-type phenotype, and both clones were found to be capable of greatly enhancing the rate of PA14 RSCV reversion to the wild-type phenotype. Restriction digest profiles obtained with EcoRI, PstI, and HindIII restriction enzymes showed the presence of a cosmid with the same insert in both cases, which was designated pED20. Although the PA14 RSCV phenotype was normally very stable in liquid culture (i.e., no wild-type revertants observed when an overnight culture was plated on LB agar), the majority of the cells in a PA14 RSCV culture carrying pED20 formed wild-type colonies after overnight incubation.

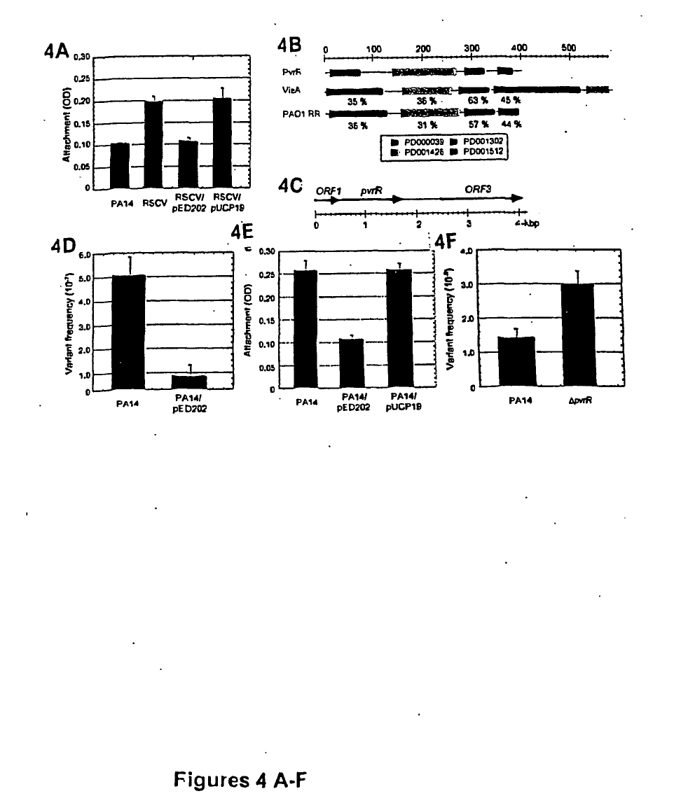

Cosmid pED20 was then subcloned into the pUCP19 plasmid vector using a PstI restriction digest. The clones obtained after transformation in E. coli were used to isolate plasmid DNA that was subsequently introduced into PA14 RSCV by electroporation. The resulting clones were screened visually for colonies showing wild-type size and morphology. Subcloning of pED20 produced pED202, which contained a 3.5-kb fragment, that restored the colony phenotype of PA14 RSCV variant to wild-type. Clone pED202 restored attachment phenotypes (FIG. 4A), as well as the colony morphology and autoagglutination phenotypes of PA14 RSCV variants to wild-type. The vector alone did not have any effect on the phenotypes analyzed.

DNA sequencing and sequence analysis of the pED202 insert was then performed. The DNA fragments used for sequencing were PCR amplified initially using primers M13 and M13 reverse from the pUCP19 plasmid. Primers were later synthesized based on the sequencing data obtained. Sequencing data were analyzed using the DNAStar software (DNASTAR Inc., Madison, Wis.) to predict the open reading frames present in the pED202 3.5 kb insert. Sequence information was also compared with the sequence databases at the National Center for Biotechnology Information as well as to the P. aeruginosa PAO1 sequence generated by the P. aeruginosa genome project (Cystic Fibrosis Foundation and PathoGenesis Corporation).

Analysis of the sequencing data obtained from clone pED202 showed that the clone contained only one intact open reading frame. The nucleotide and predicted amino acid sequences of the ORF (designated pvrR for phenotype variant regulator) contained in clone pED202 were compared to the GenBank databases, and showed sequence similarities to response regulator elements of the two-component regulatory system. The search revealed 30% identity and 45% similarity in a 376 amino acid overlap to the Vibrio cholerae response regulator VieA, which is induced during intestinal infection in mouse. In addition, the ORF on pED202 showed 29% identity and 45% similarity to a probable two-component response regulator identified in P. aeruginosa strain PAO1 (ORF PA3947). Interestingly, the region of the PA14 genome containing pvrR is not present in the fully sequence P. aeruginosa strain PAO1.

A homology search against domain sequences in the ProDom database (ProDom web site; http://prodes.Toulouse.inra.fr/prodom) identified 4 regions with high-scoring segment pairs in PvrR (FIG. 4B). All 4 domains are also present in VieA and the PA01 putative response regulator (FIG. 4B). Moreover, these 4 domains exhibit high levels of amino acid sequence similarity (30%-60%; FIG. 4B). Sequence analysis of the regions located upstream and downstream of pvrR revealed the presence of two additional ORFs (designated ORF1 and ORF3 respectively; FIG. 4C) with sequence homology to two-component regulatory elements.

The protein encoded by ORF1 has homology to probable sensor/response regulator hybrids from P. aeruginosa (35% identity and 49% similarity to ORF. PA2824), to the sensor protein RcsC (capsular synthesis regulator component C) from Salmonella enterica subsp. enterica serovar Typhi (30% identity and 51% similarity) and to a two-component sensor regulator (PheN) that modulates phenotypic switching in P. tolaasii, (31% identity and 45% similarity). The protein encoded by ORF3 shows 42% identity and 60% similarity to the GacS sensor kinase from P. fluorescens, and 41% identity and 59% similarity to the two-component sensor regulator that modulates phenotypic switching in P. tolaasii (PheN).

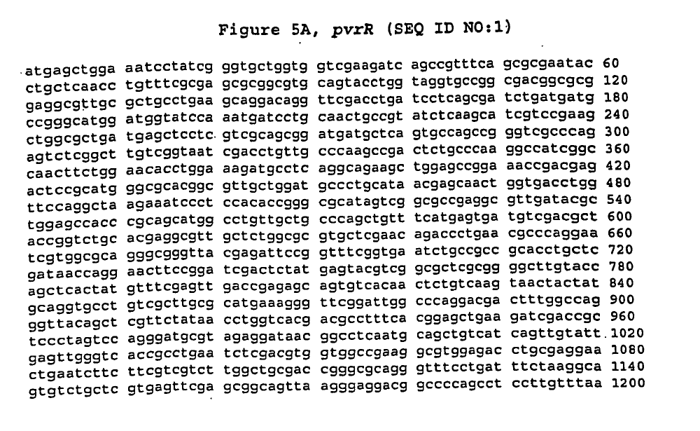

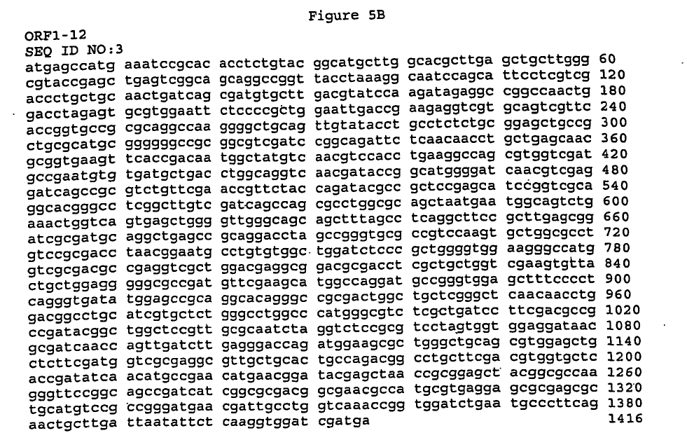

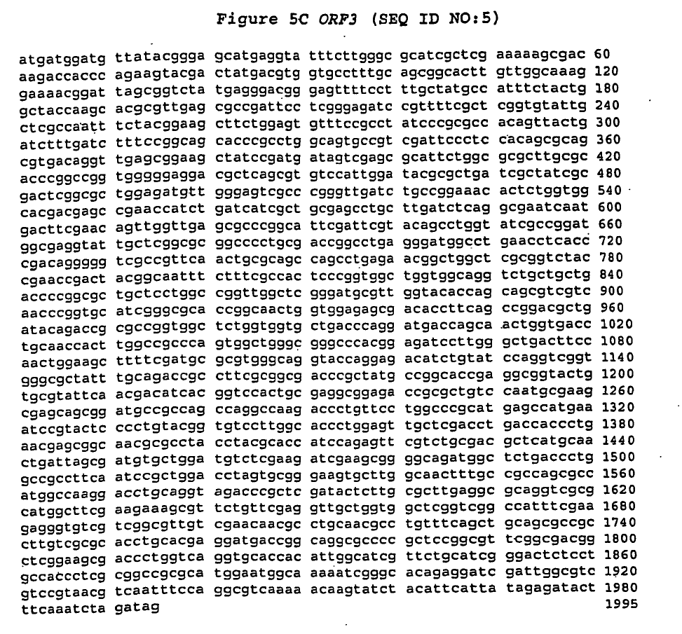

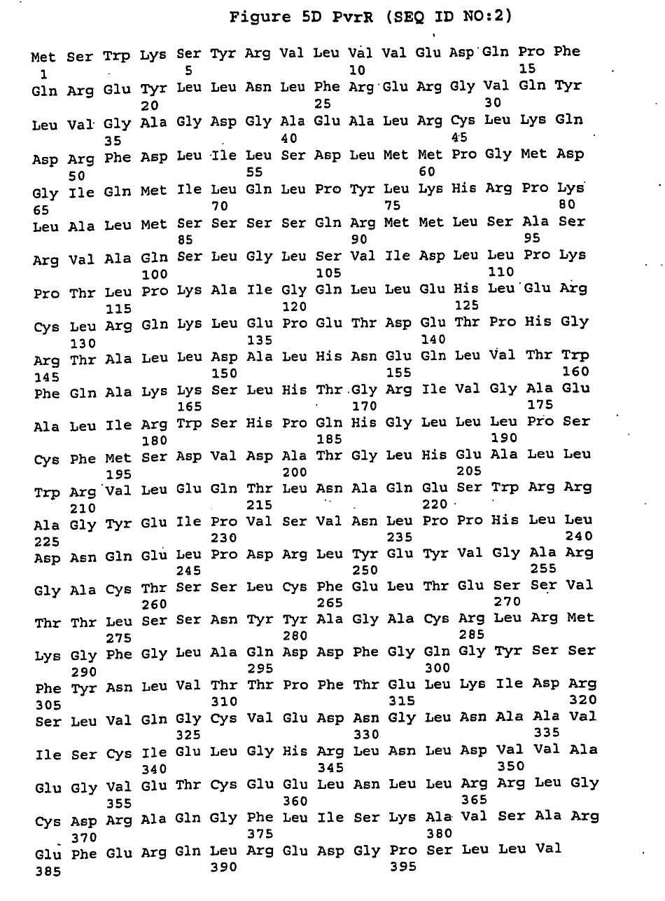

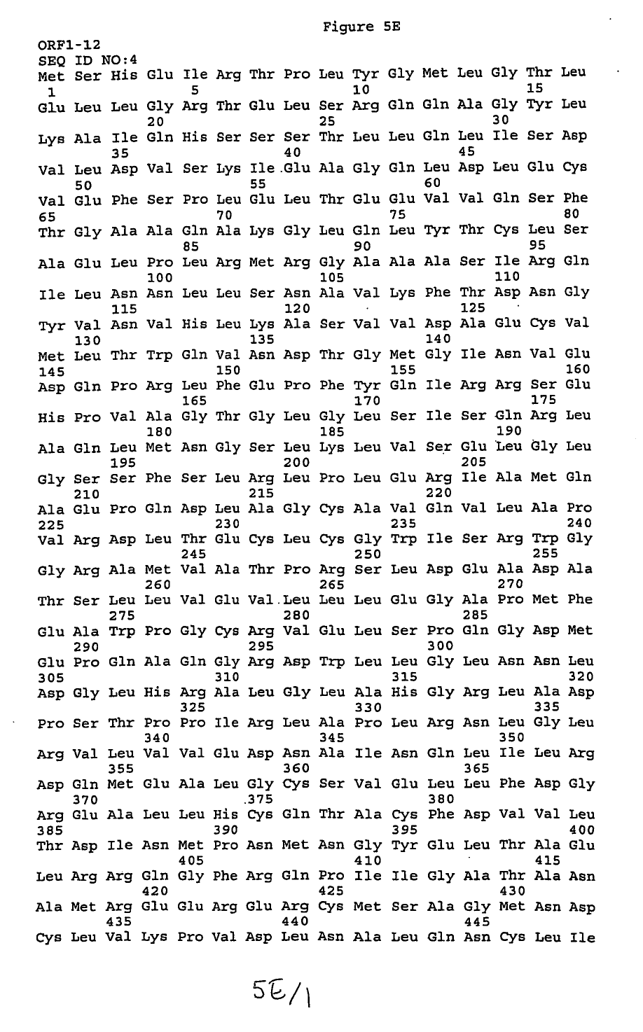

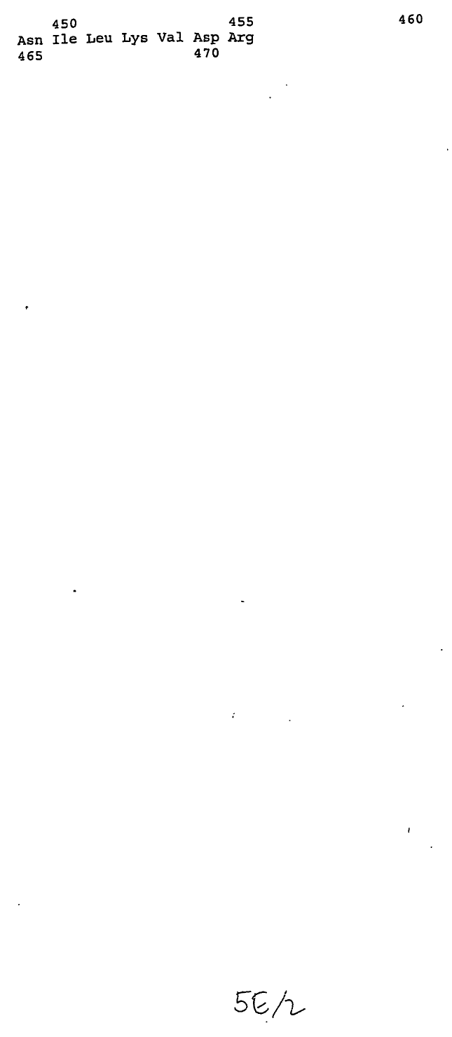

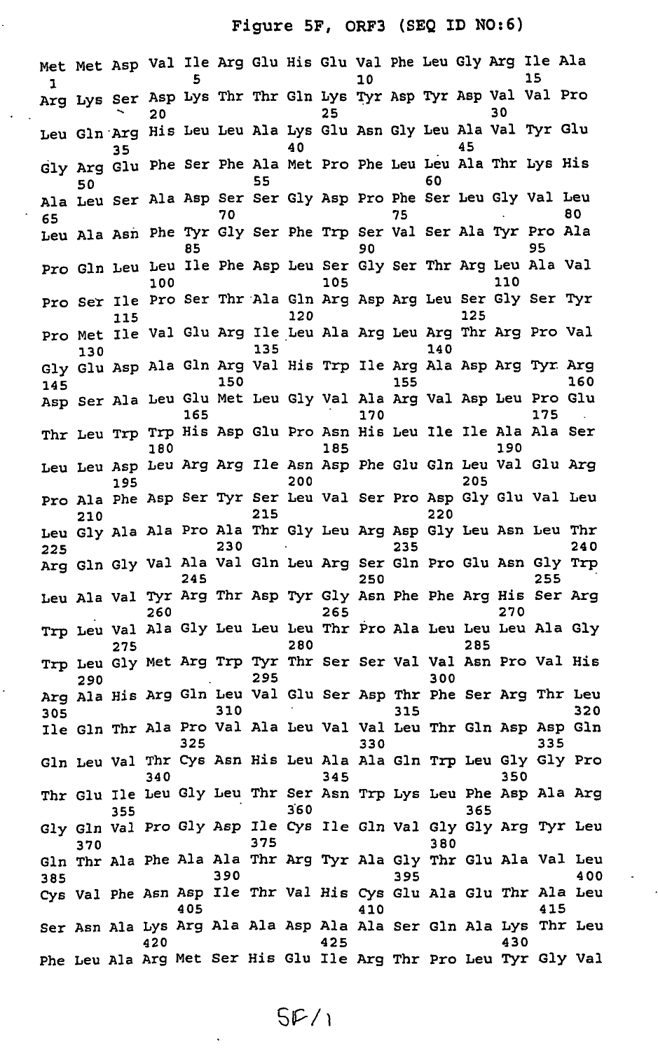

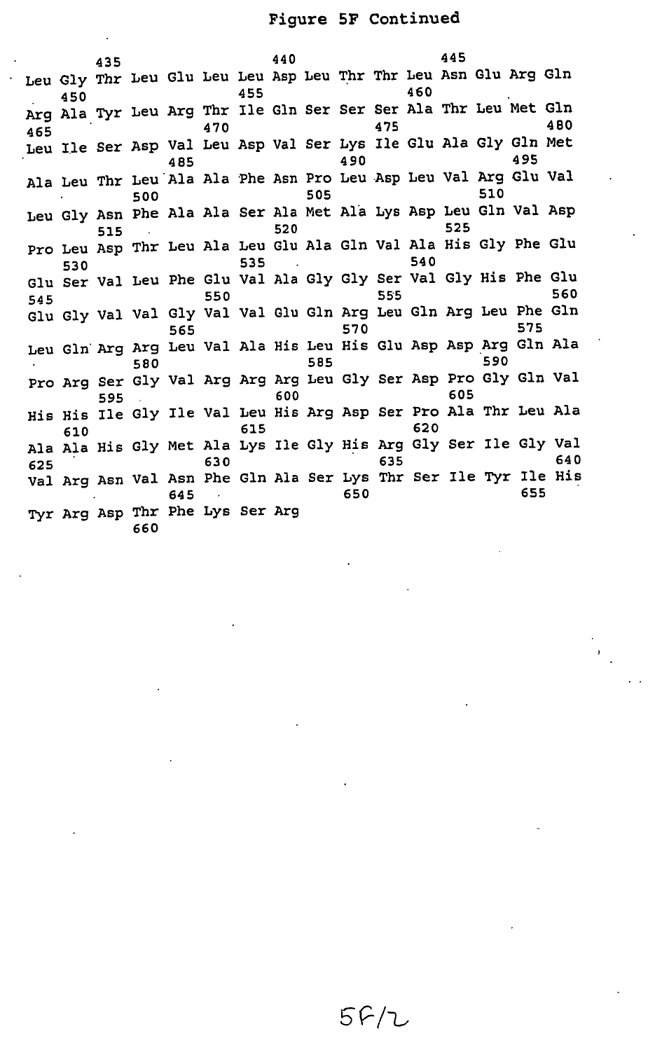

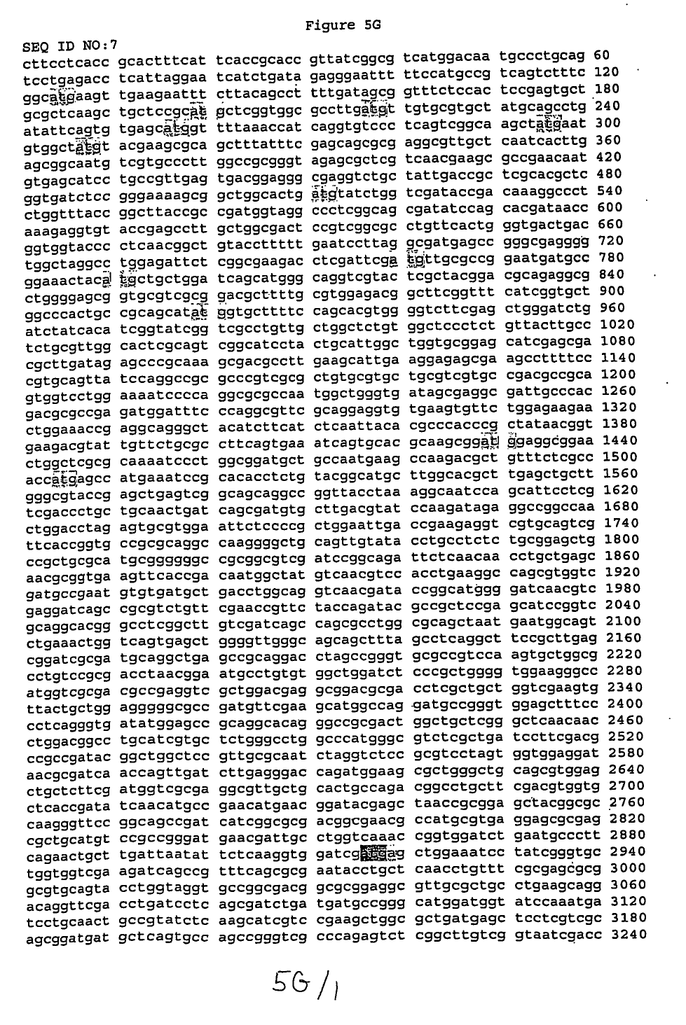

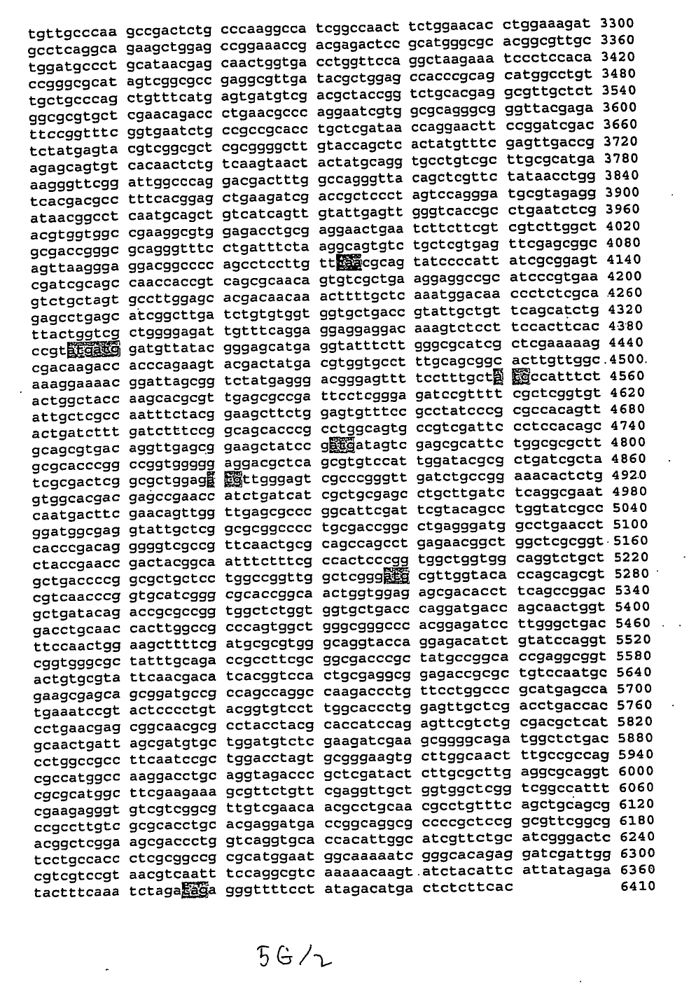

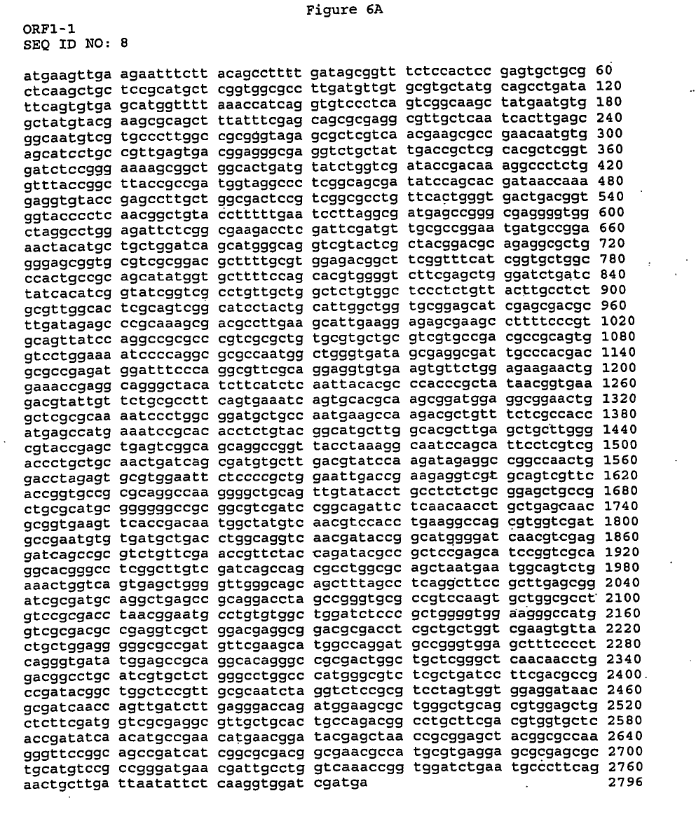

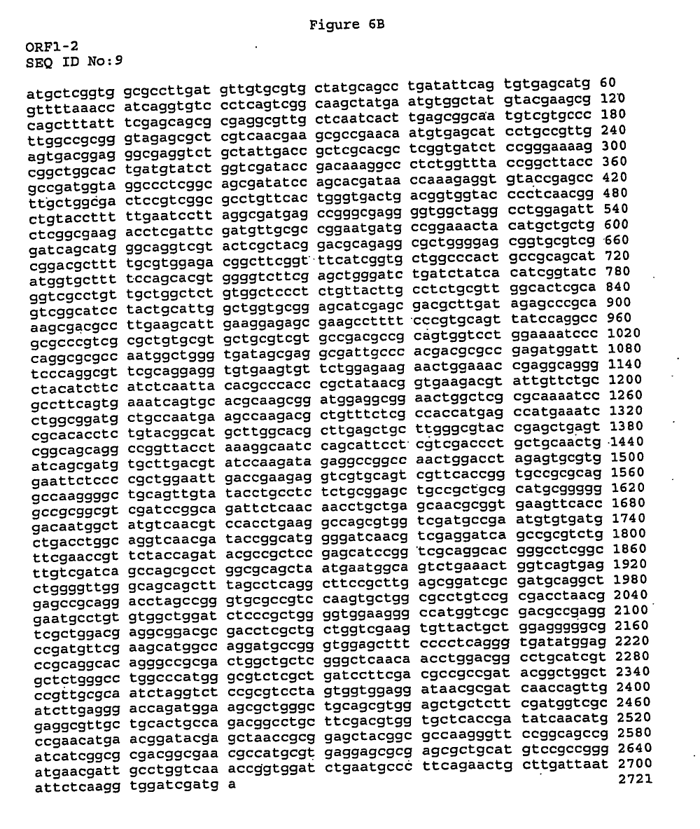

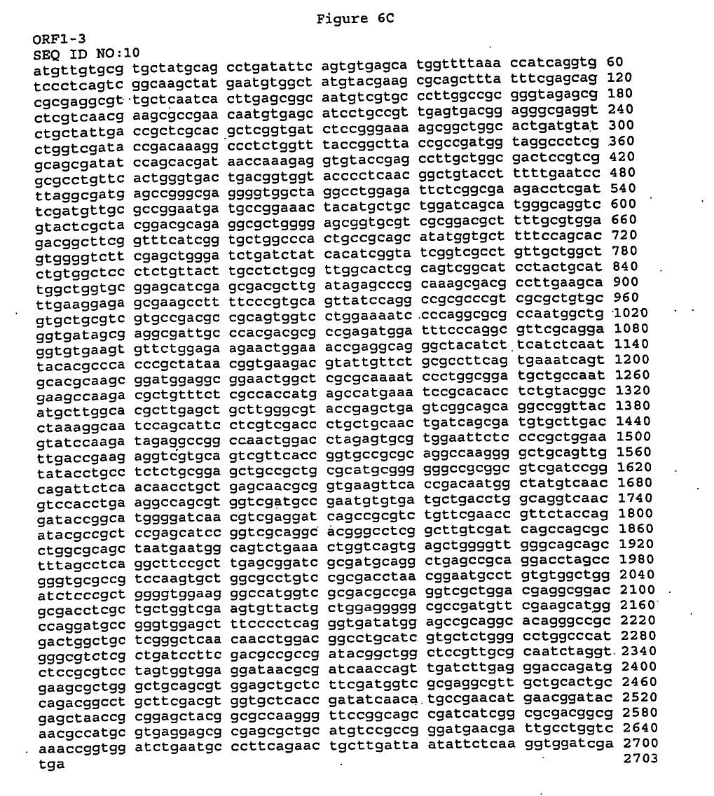

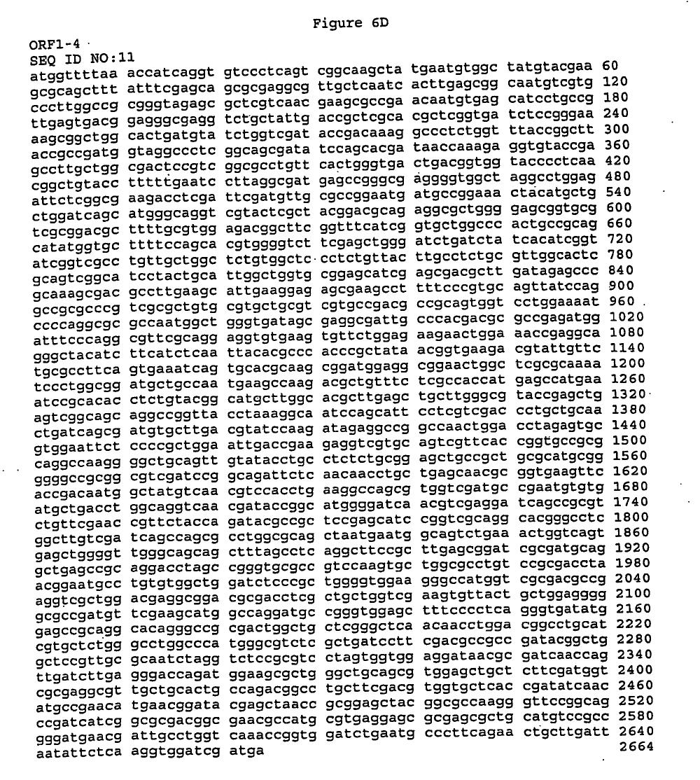

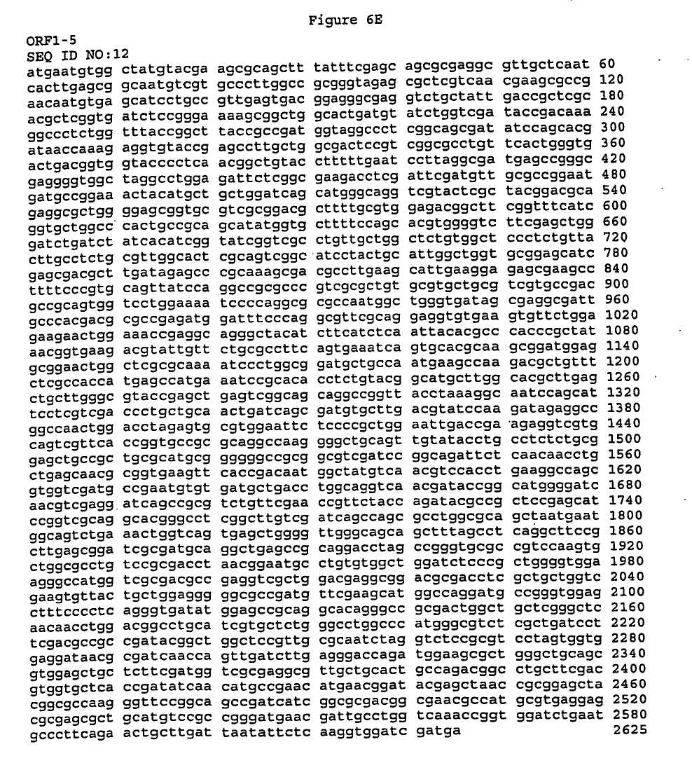

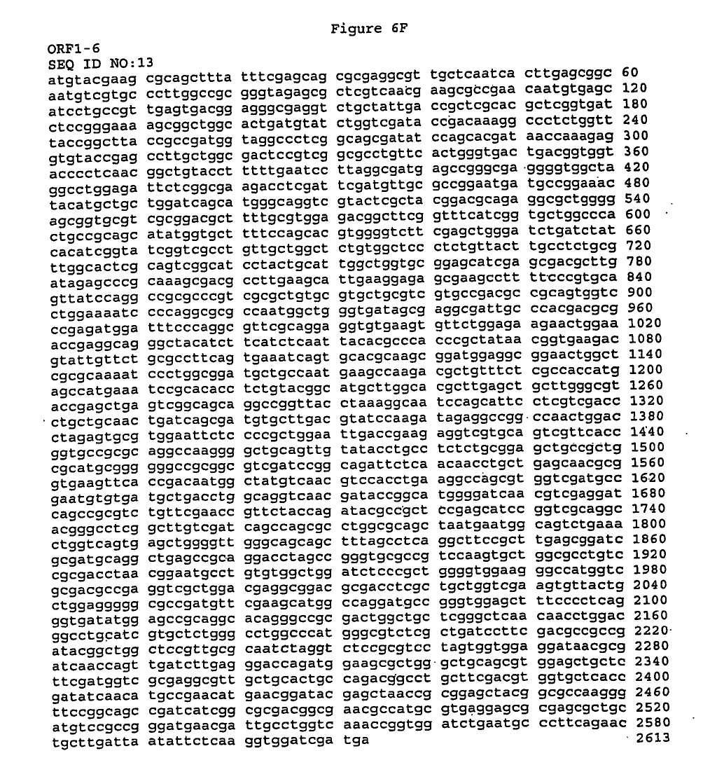

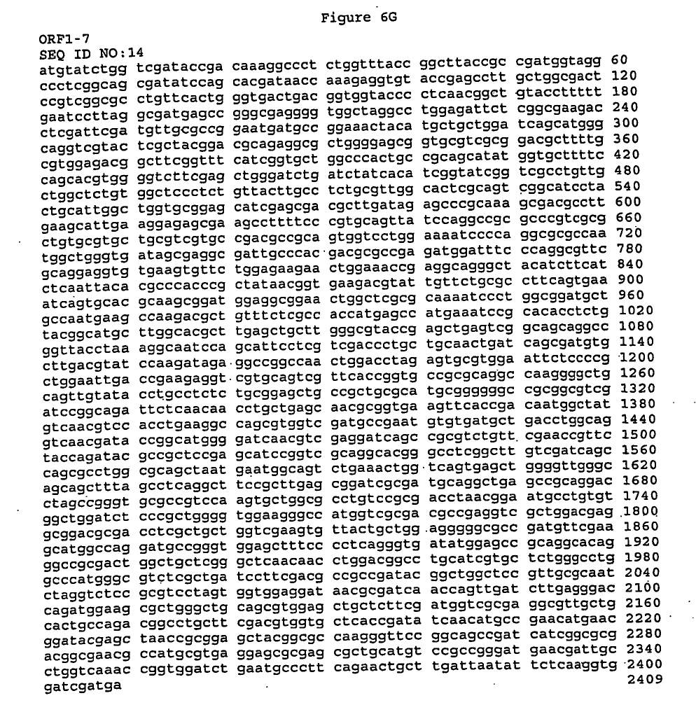

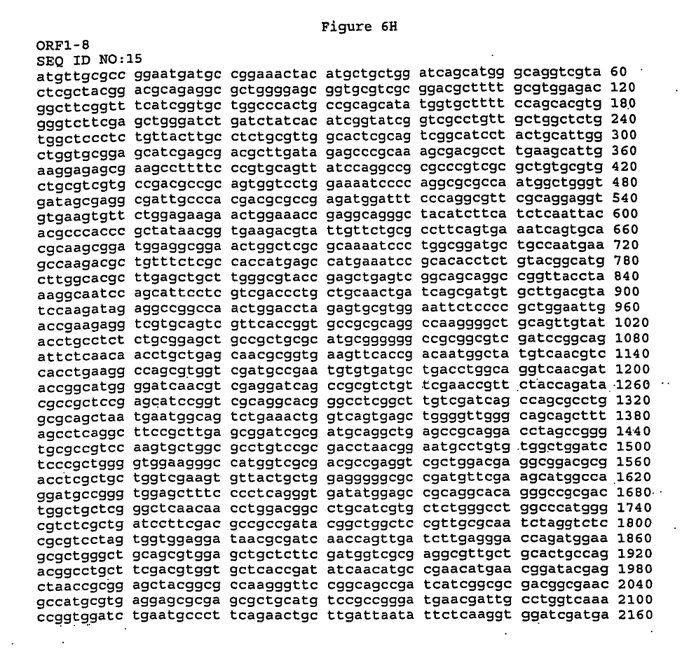

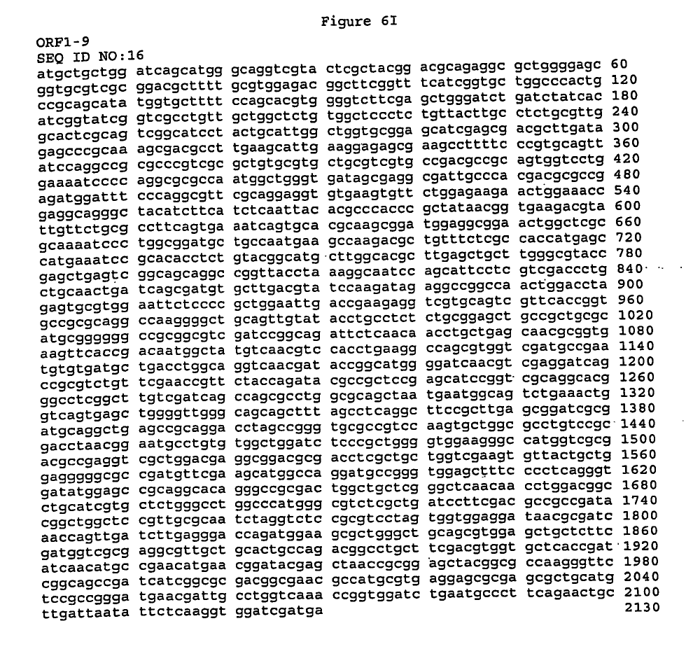

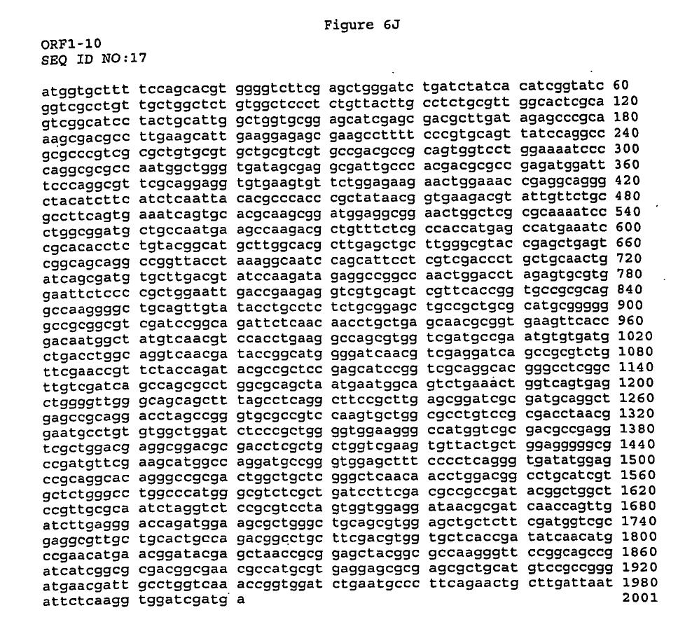

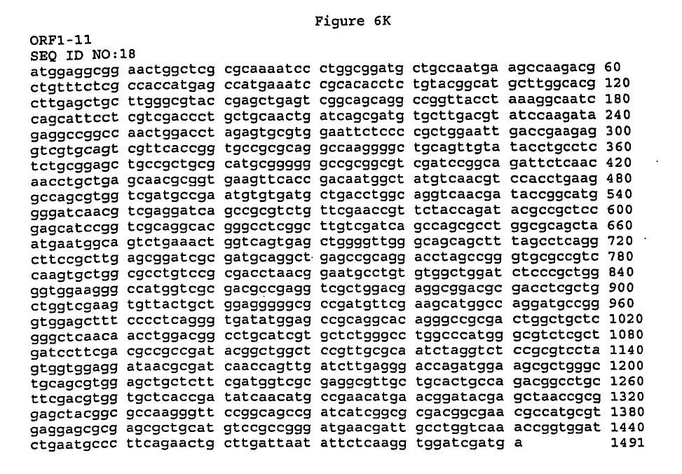

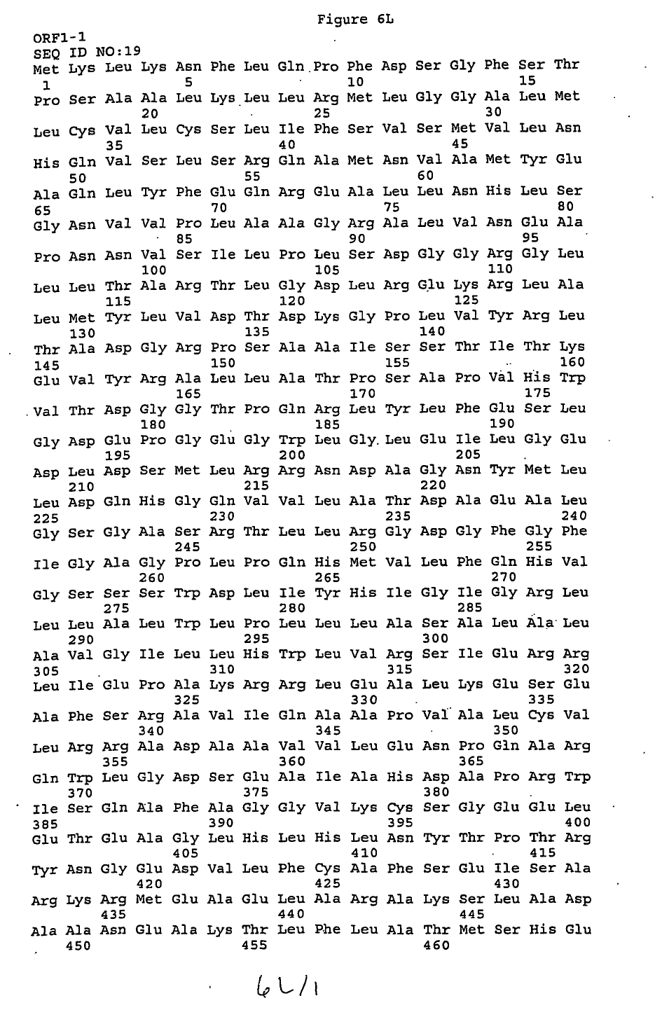

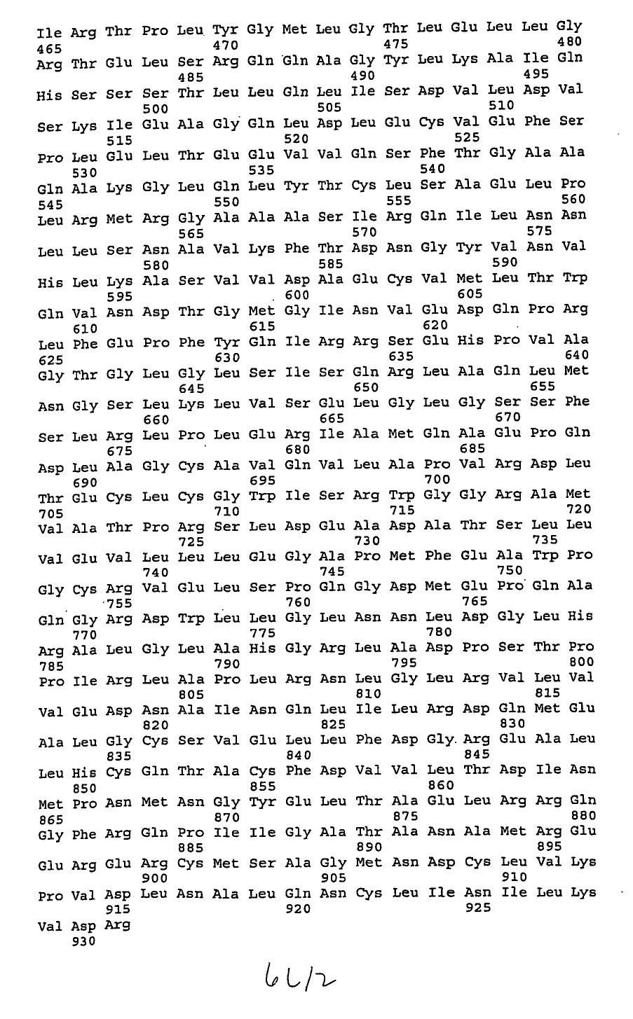

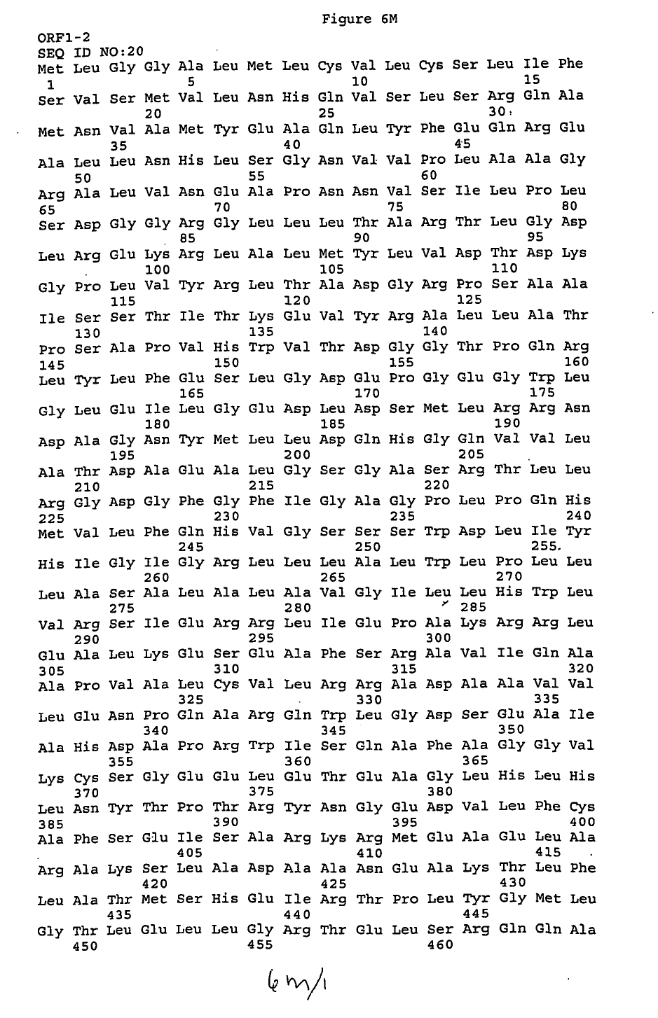

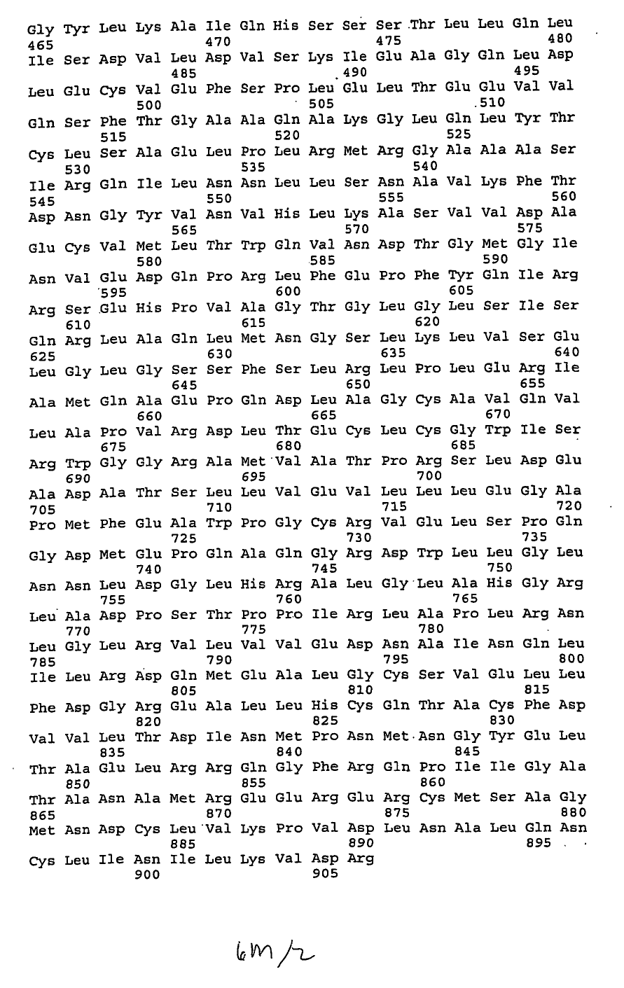

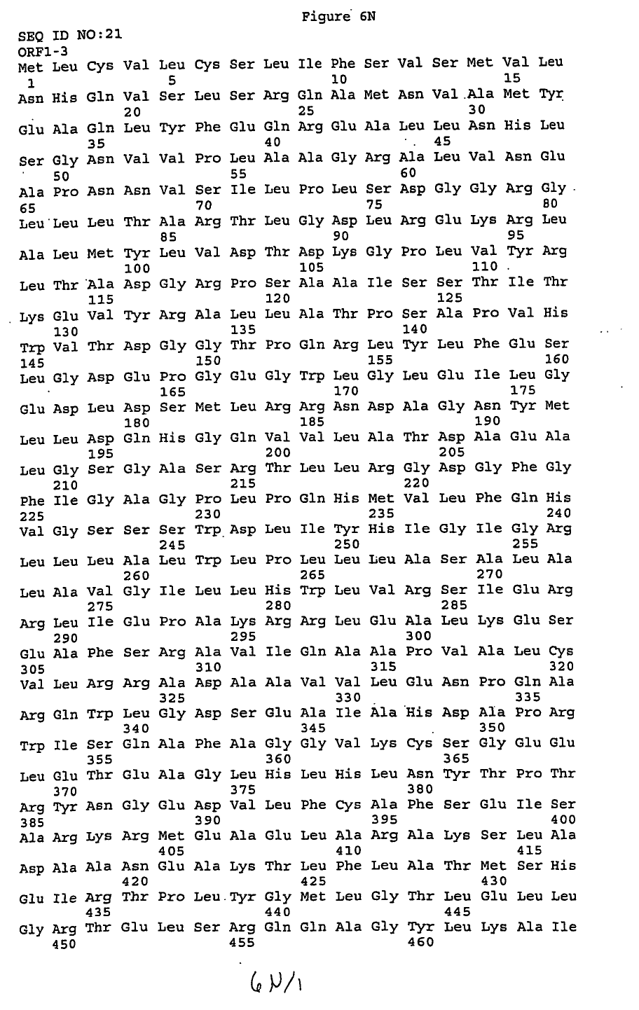

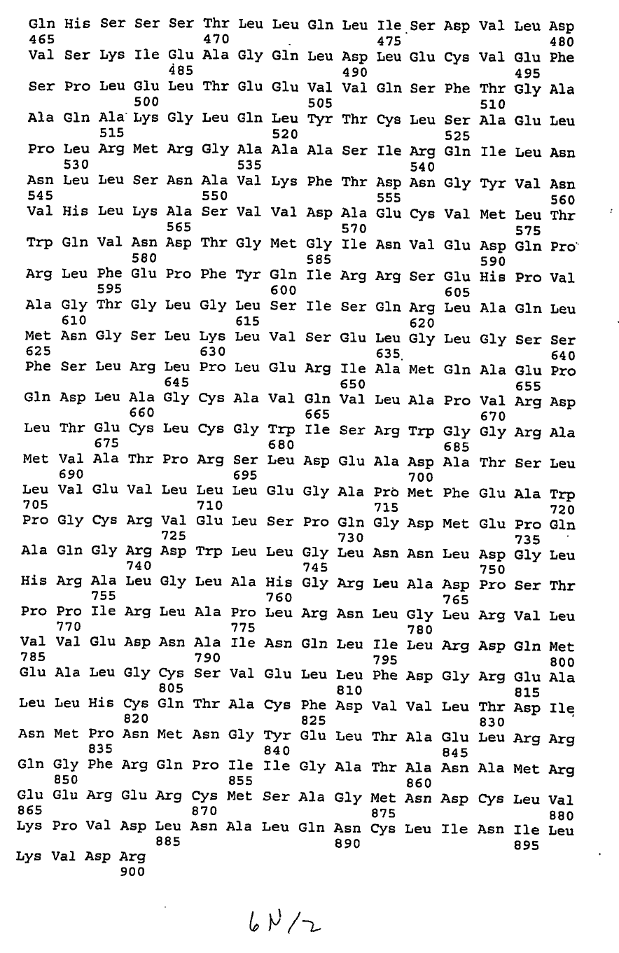

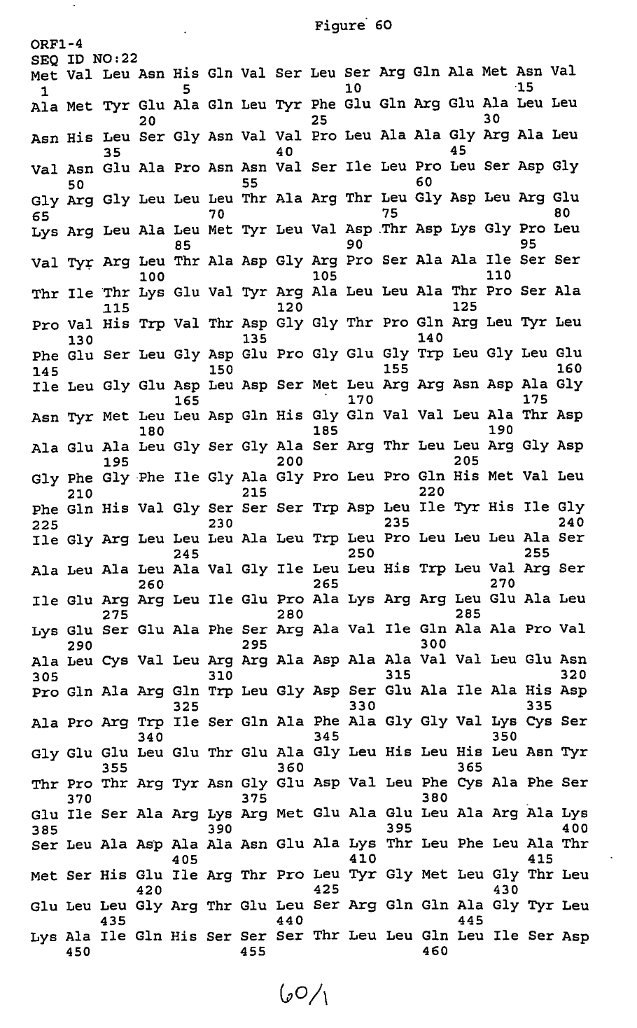

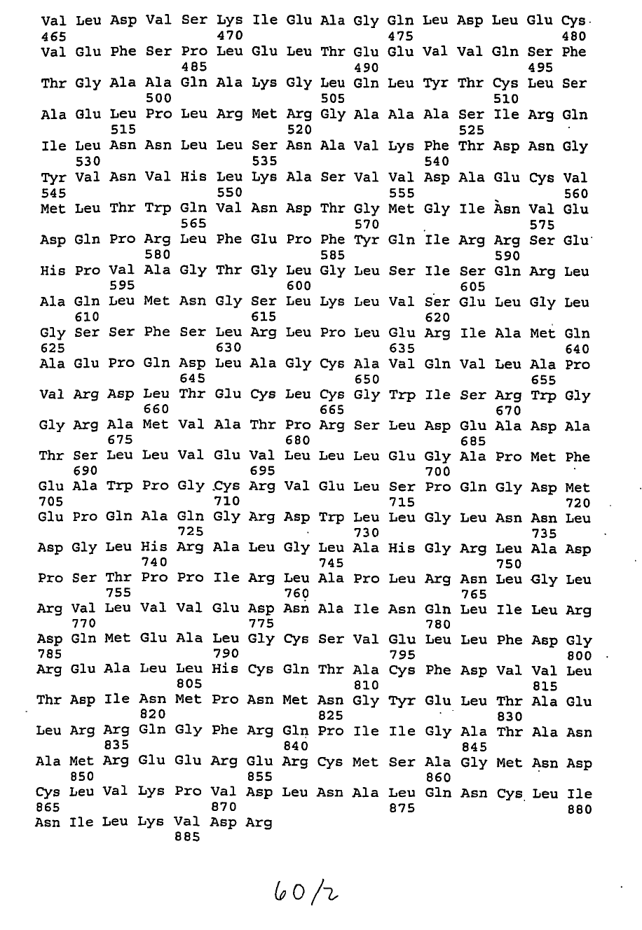

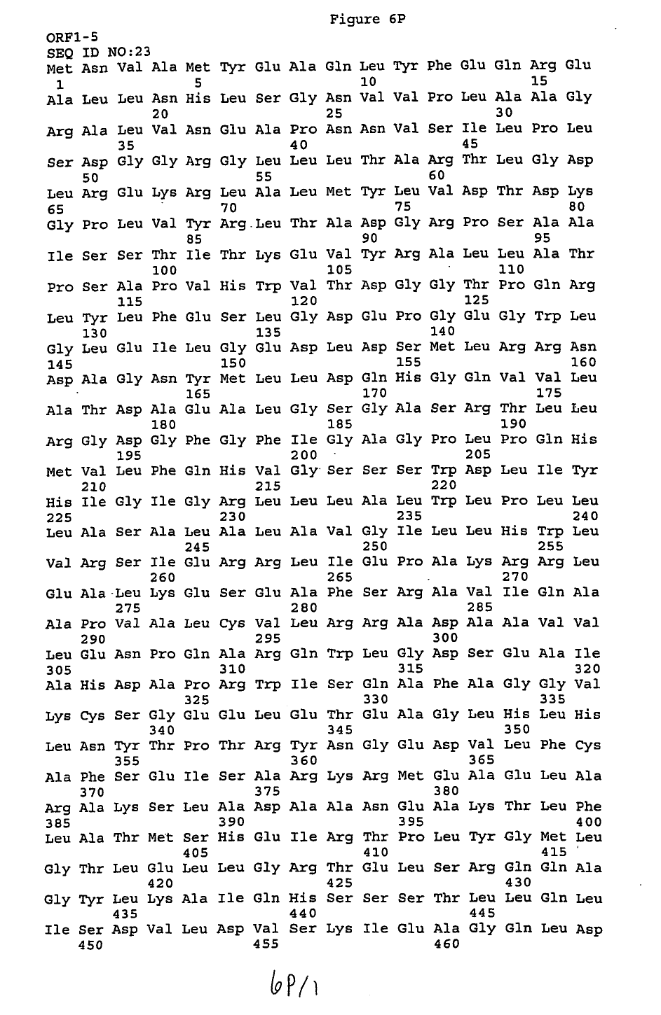

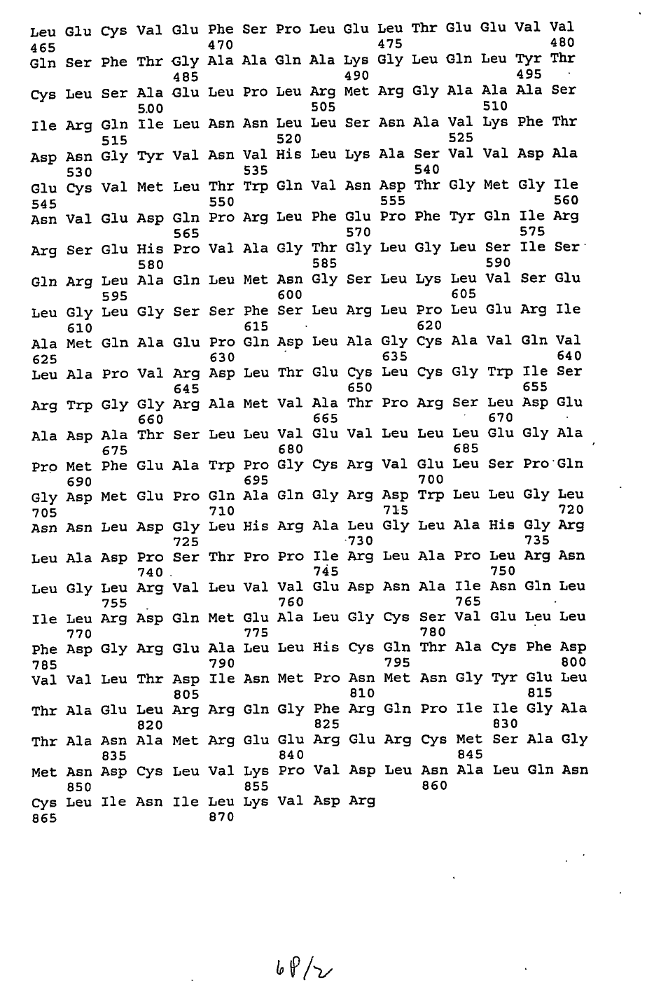

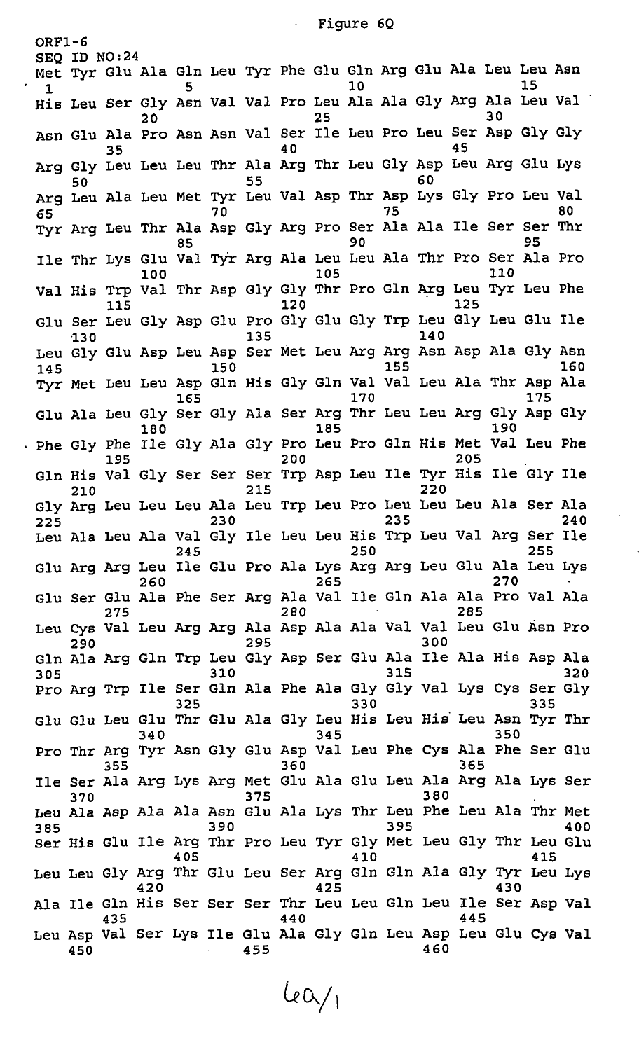

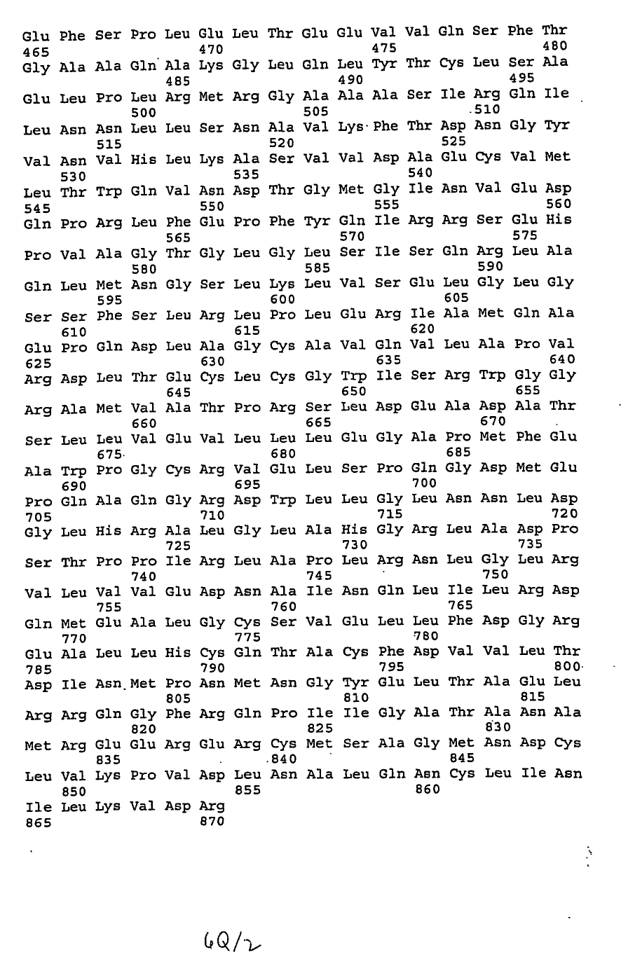

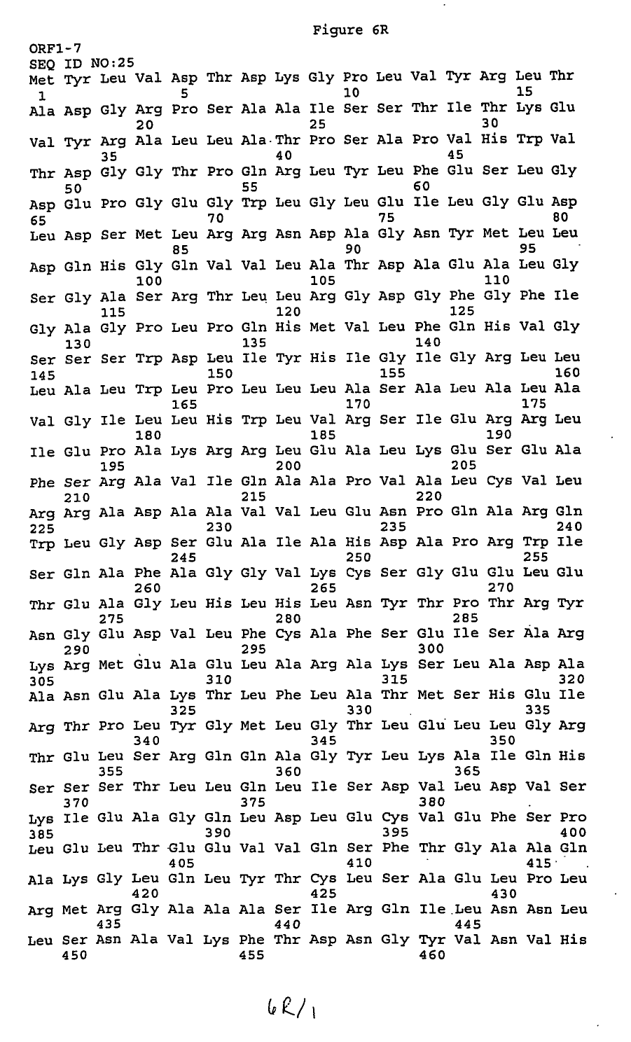

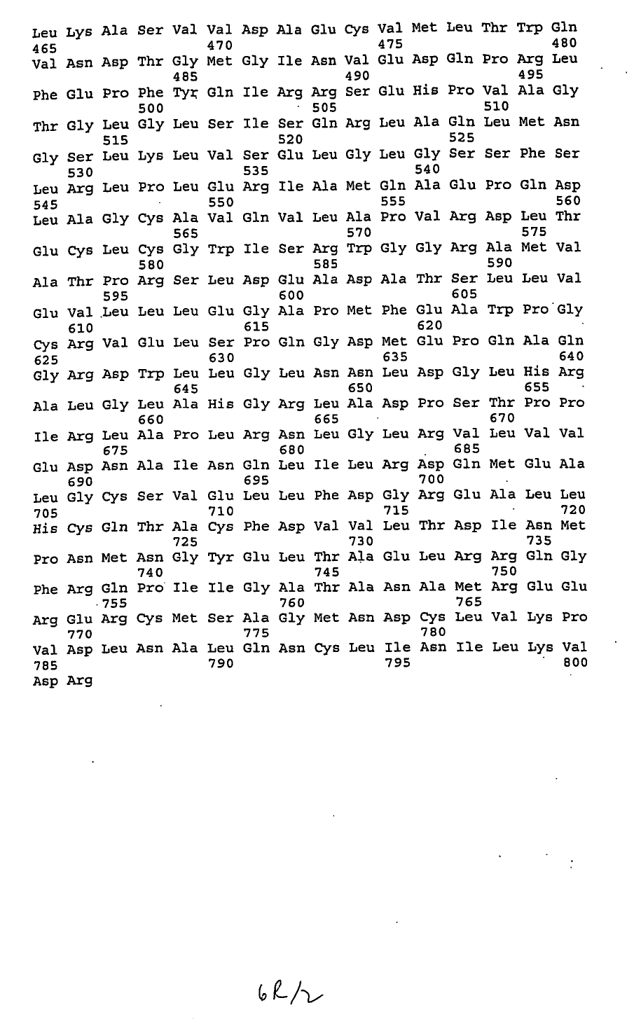

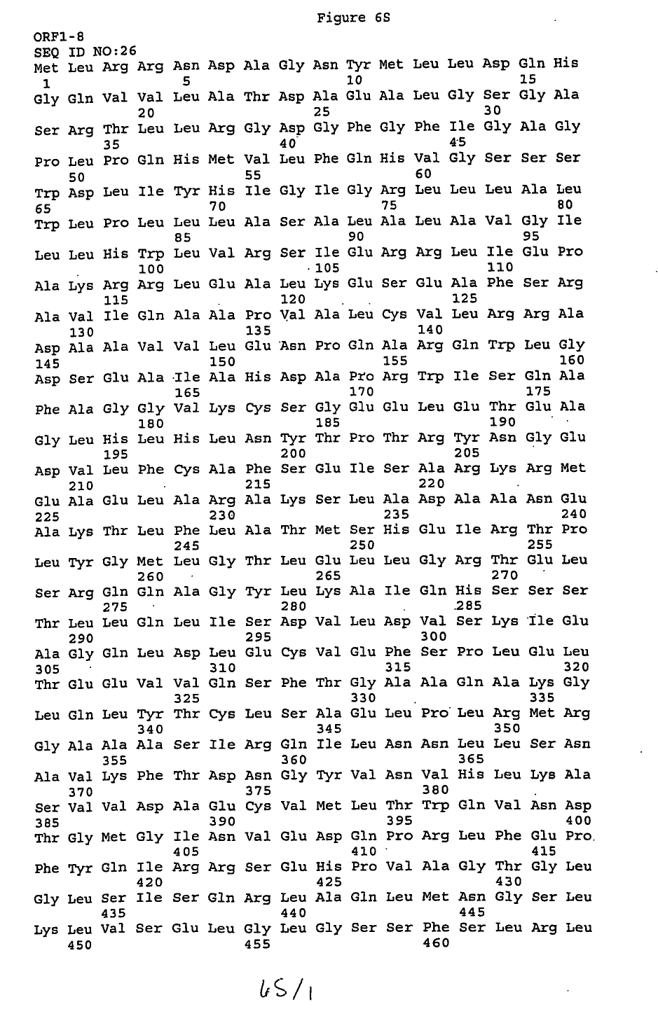

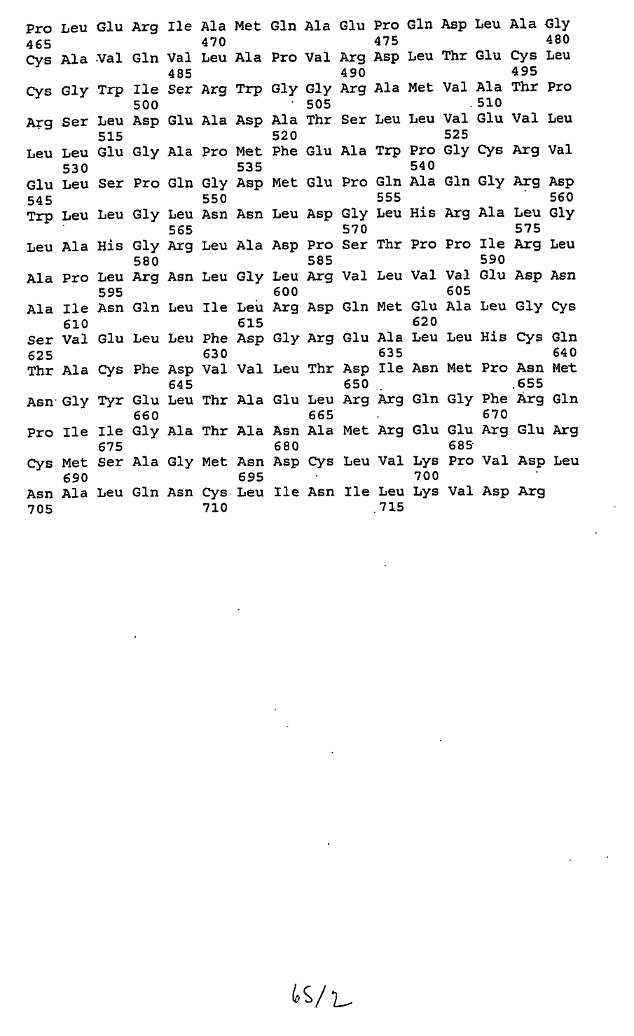

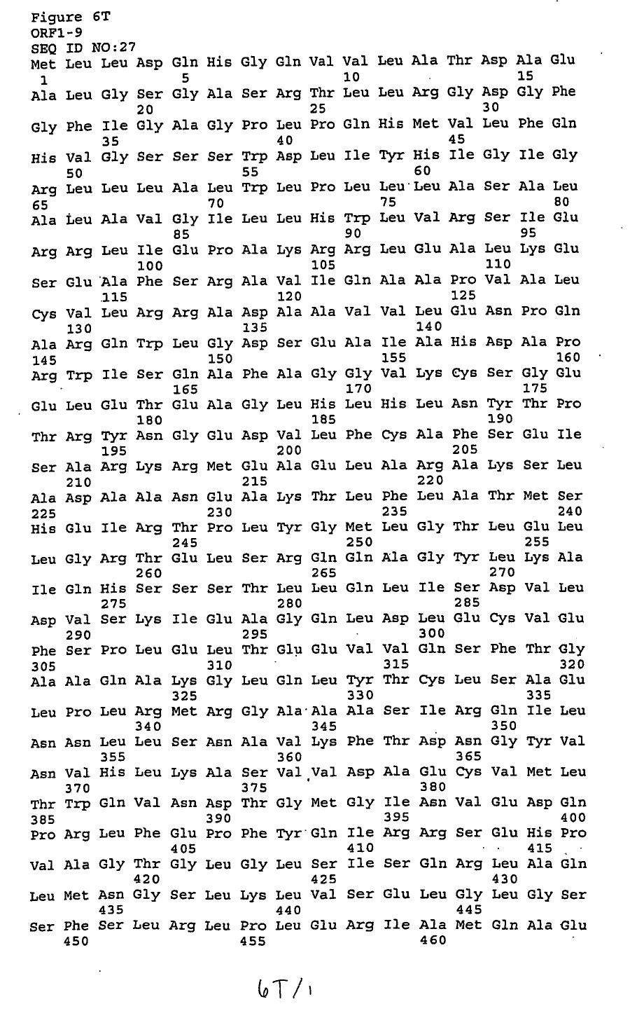

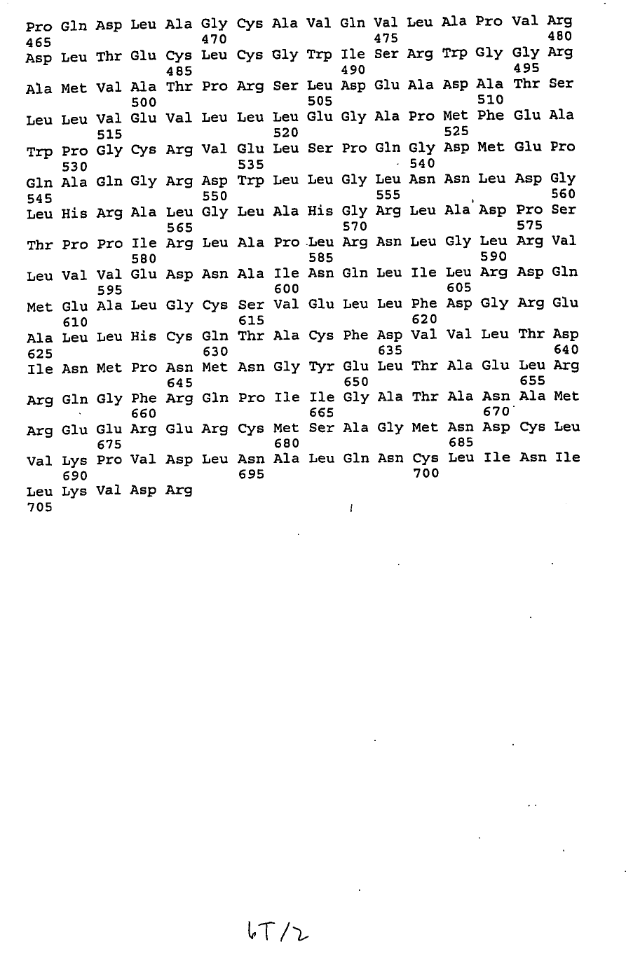

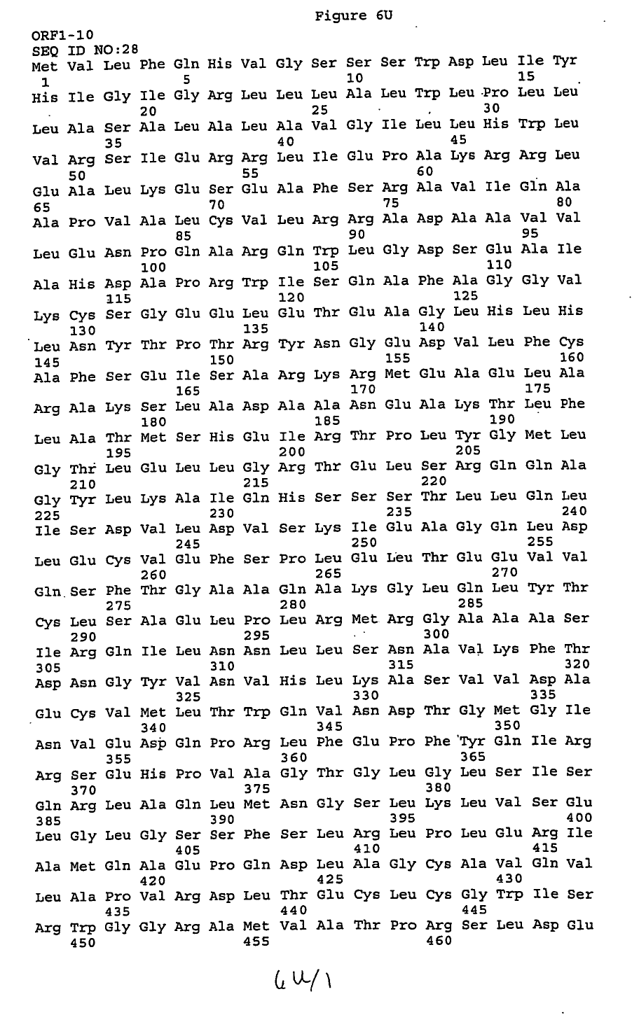

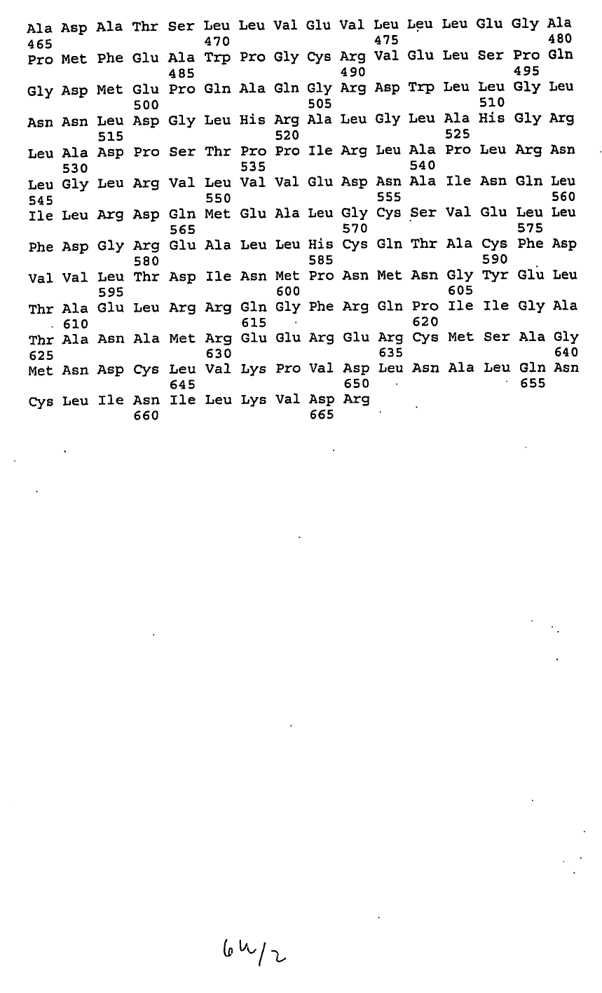

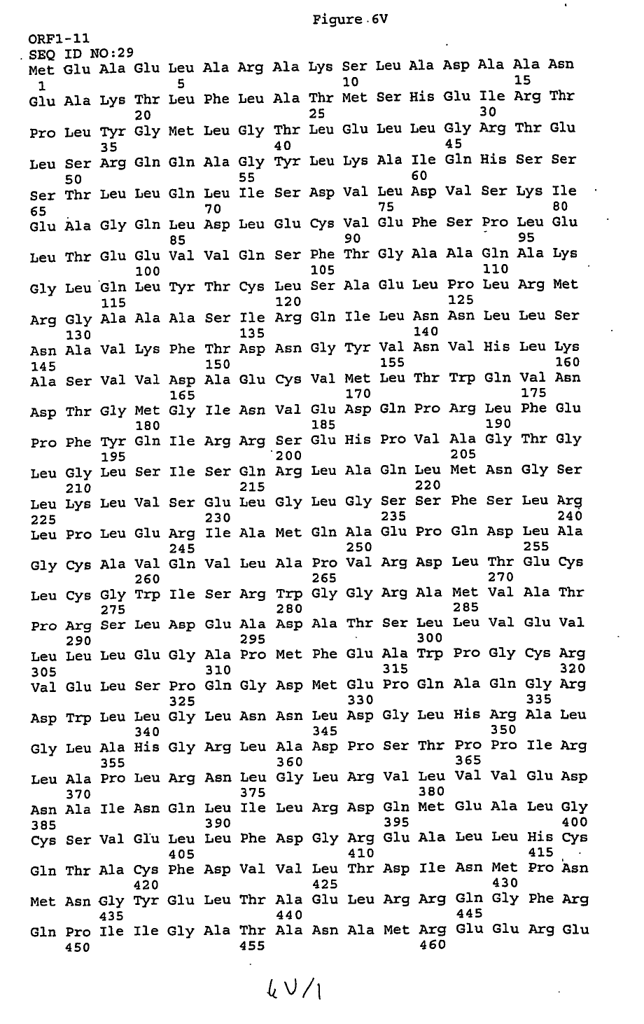

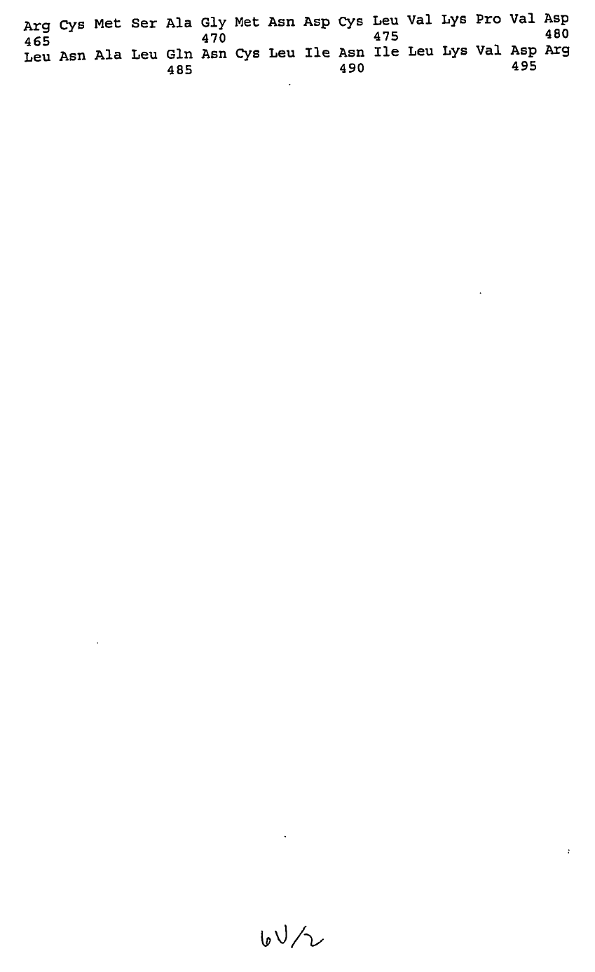

FIG. 5G shows a nucleic acid sequence (SEQ ID NO:7) including polynucleotides identified in the ORF1 region (SEQ ID NOS:3, and 8-18), pvrR (SEQ ID NO:1), polynucleotides identified in the ORF3 region (SEQ ID NOS:5, and 30-34), and the intergenic regions. The start and stop codons for each open reading frame are indicated by highlighting. FIGS. 5B and 6A-K show the nucleotide sequences of several open reading frames identified in the ORF1 region. The deduced amino acid sequence of these open reading frames are shown in FIGS. 5E (SEQ ID NO:4) and 6L-6V (SEQ ID NOS:19-29).

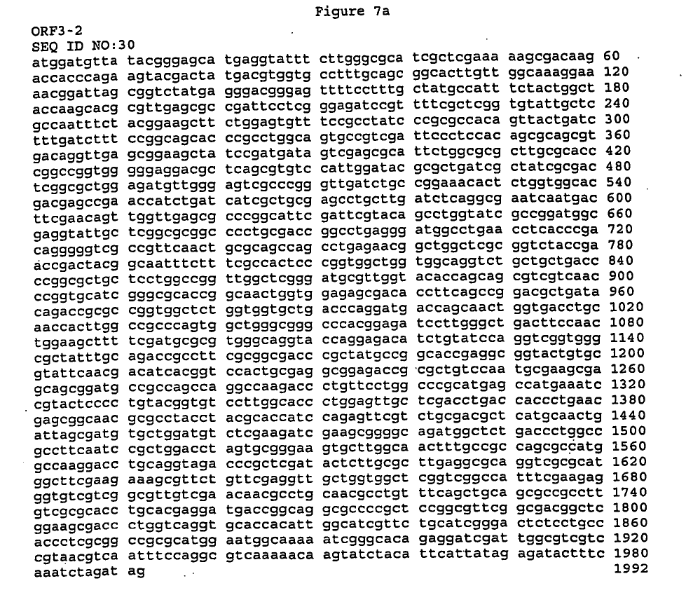

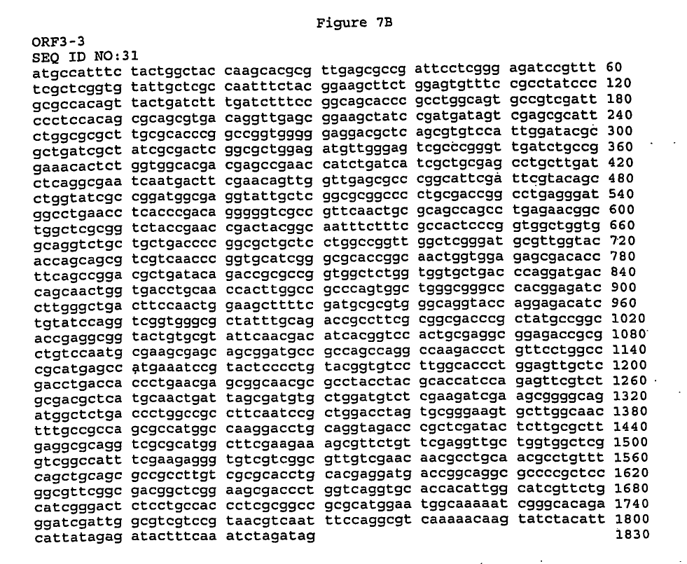

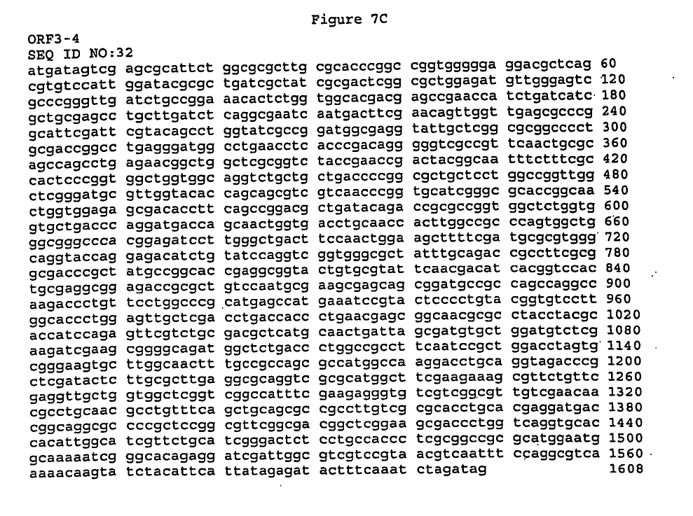

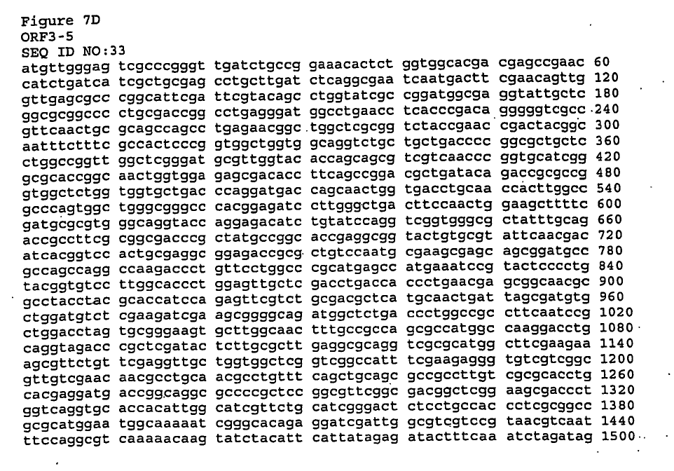

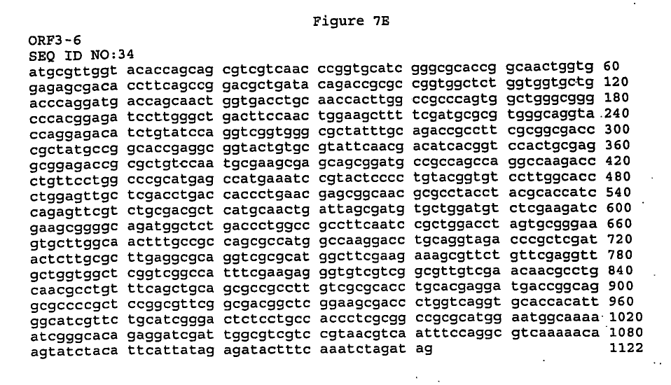

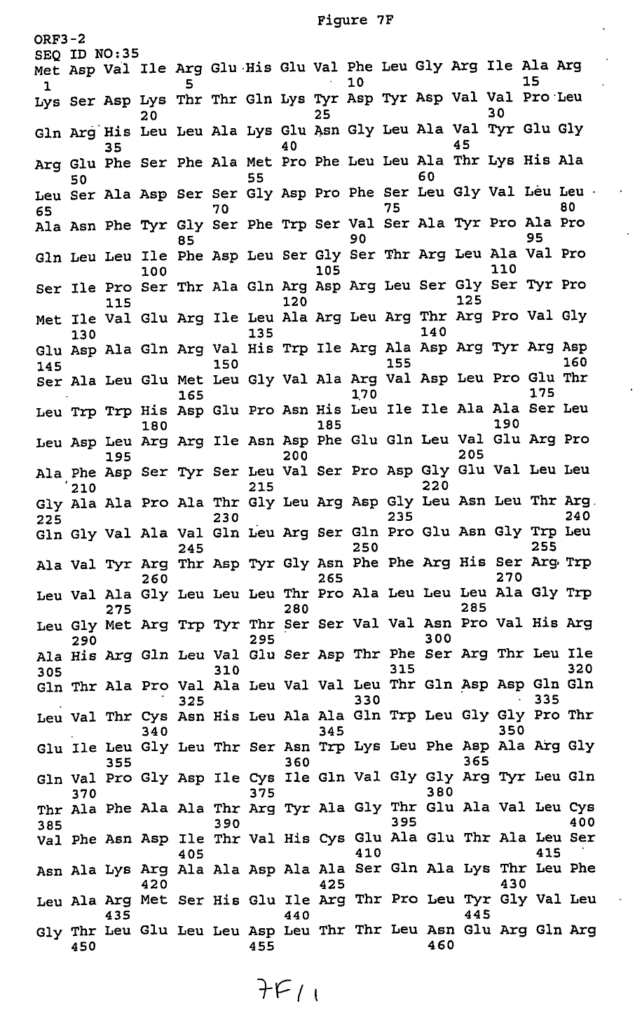

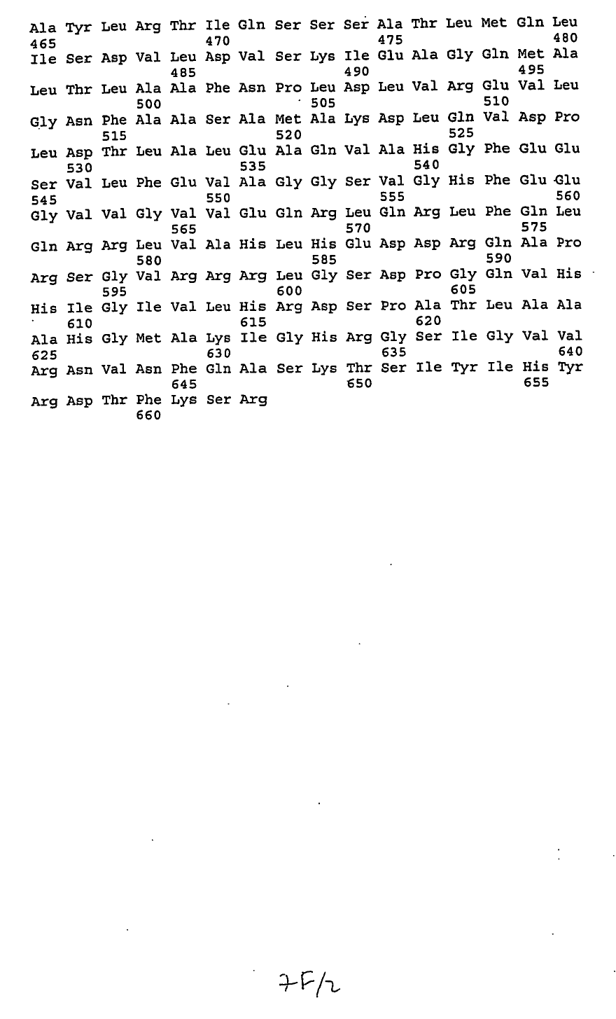

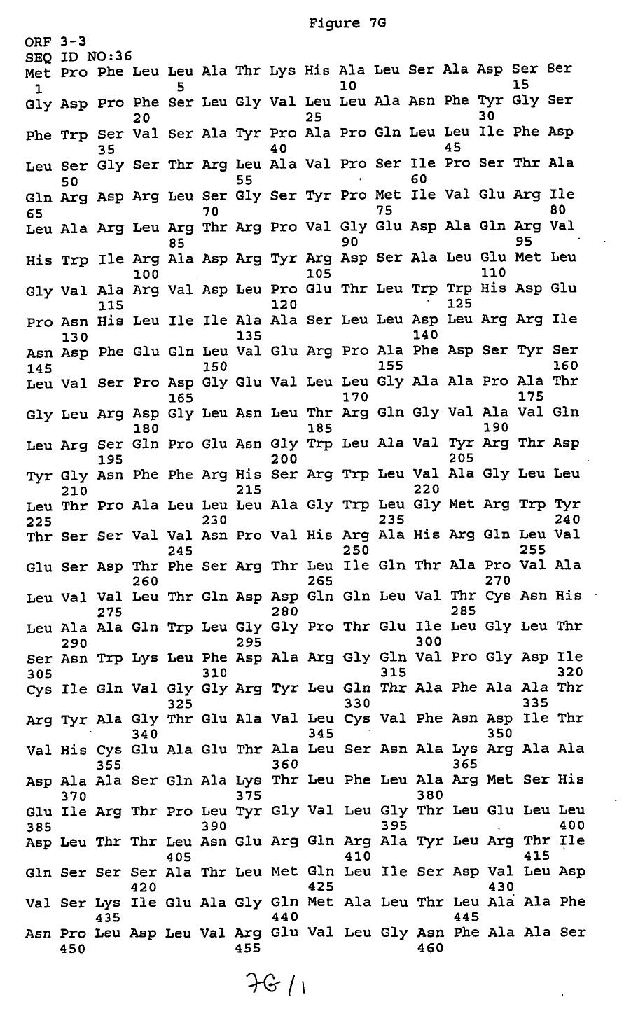

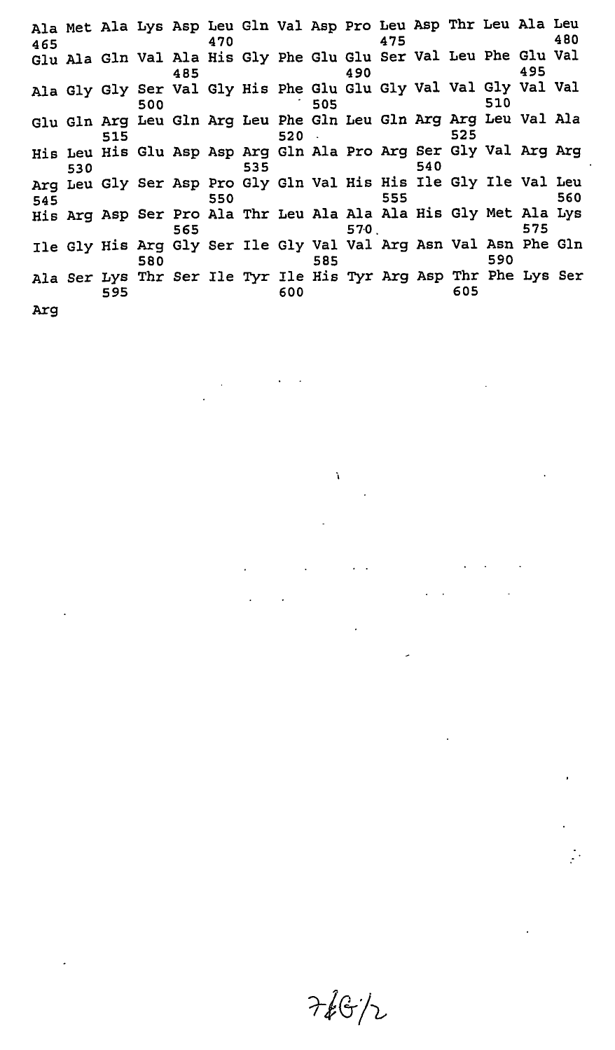

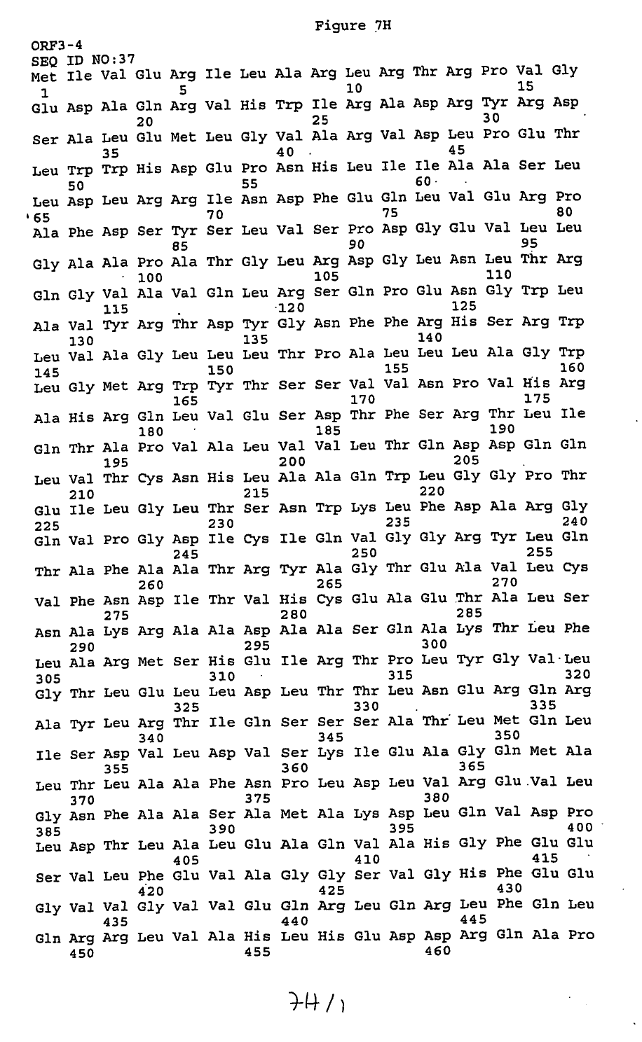

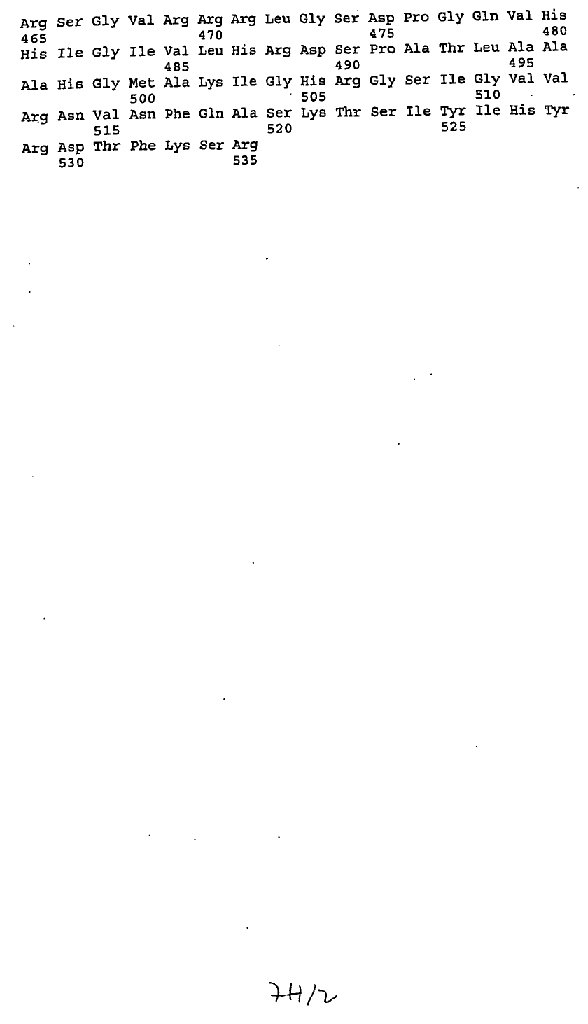

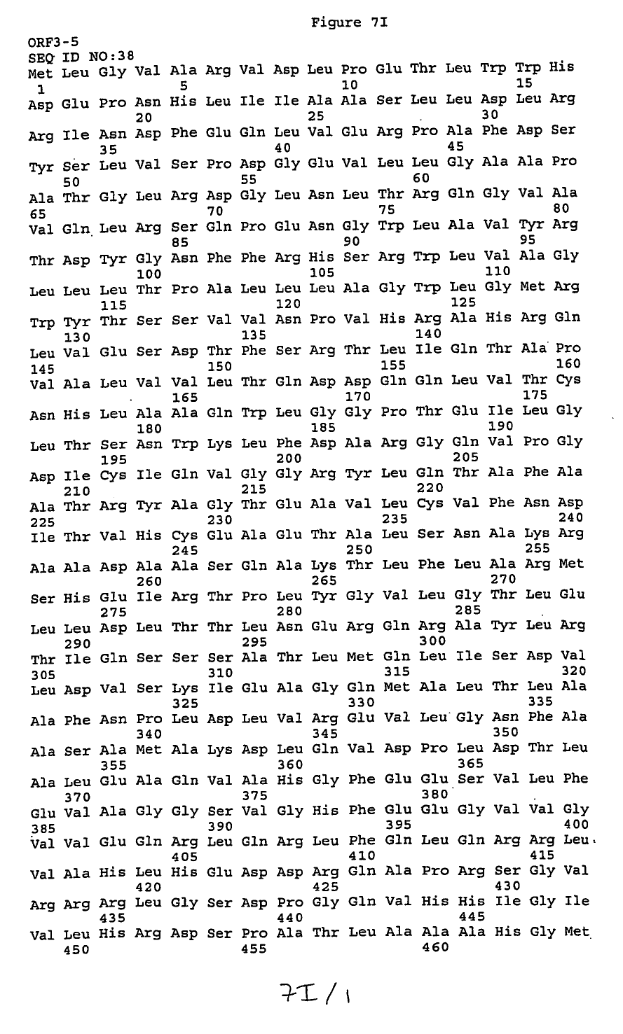

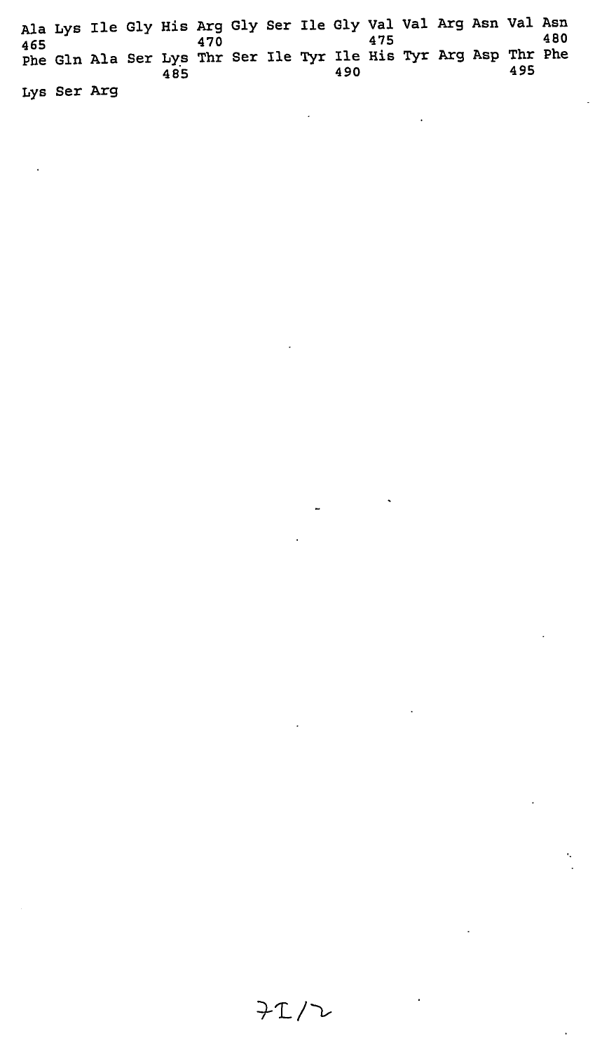

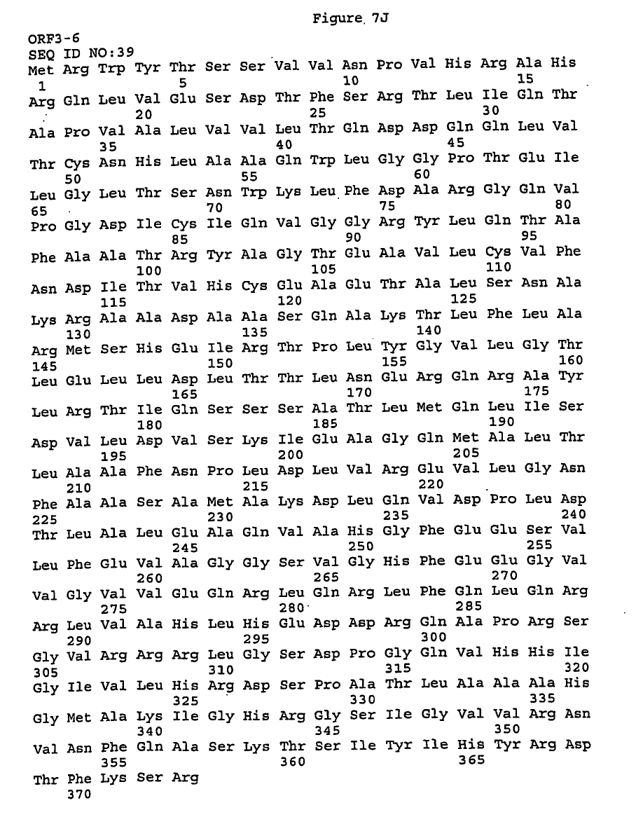

Additionally, FIG. 5C shows the nucleic acid sequence (SEQ ID NO:5) of one of several open reading frames identified in the ORF3 region. The deduced amino acid sequence of the polypeptide encoded by this nucleotide sequence is shown in FIG. 5F (SEQ ID NO:6). FIGS. 7A-7E (SEQ ID NOS:30-34) show the nucleotide sequences of several additional open reading frames identified in the ORF3 region. The deduced amino acid sequence of the polypeptides encoded by these nucleotide sequences are shown in FIGS. 7F-7J.

To determine whether pvrR or a highly similar pvrR homolog was present in the other P. aeruginosa strains, PCR analysis of 14 P. aeruginosa strains was performed using pvR-specific primers. The specificity of the PCR products obtained was subsequently confirmed by Southern blotting and hybridization with a pvrR-specific probe. Results showed that 7 out of 7 CF isolates, 2 out of 3 clinical isolates and 3 out of 4 standard P. aeruginosa laboratory strains contained the pvrR gene fragment or a highly similar fragment (data not shown).

PvrR Overexpression

Consistent with the putative role of PvrR in the regulation of phenotypic switching, overexpression of PvrR from pED202 resulted in a 6-fold reduction in the frequency of resistant variants obtained after plating overnight cultures on kanamycin (200 μg/ml) plates compared to wild-type (FIG. 4D). Plasmid pED202, containing the pvrR gene was introduced into wild-type PA14 by electoroporation using standard methods. Frequency of appearance of kanamycin resistant variants and attachment to 96-well PVC plates was assayed as described above. Interestingly, the PvrR overexpressing strain also caused a 2.5-fold reduction in attachment to PVC plastic with respect to the strain carrying the vector alone (FIG. 4E).

PvrR Deletion Analysis

Since PvrR is involved in the regulation of the phenotypic switch from wild-type to phenotypic variant, a mutation in pvrR would be expected to alter the proportion of resistant variants present in the PA14 population. To test this hypothesis, a 914 bp in-frame deletion within pvrR (denoted “ΔpvrR”) was generated by replacing 2.33 kb of the wild-type sequence of the pvrR gene with a 1.416 kb fragment amplified by PCR. The PCR-amplified DNA fragment was subcloned into the XbaI and SmaI restriction sites of the positive selection suicide vector pCVD442 to generate pED167. Plasmid pED167 was then used in an allelic exchange procedure to introduce the fragment containing the deleted copy of pvrR into the homologous region of the PA14 chromosome, creating strain ED78. The deletion was confirmed by sequencing a PCR fragment containing pvrR.

This deletion of pvrR (ΔpvrR) in PA14 resulted in an increased frequency of appearance of resistant variants on kanamycin plates with respect to the wild-type (FIG. 4F), confirming the involvement of pvrR in the regulation of phenotypic switching. The observation that 100% of the variants expressing wild-type pvrR reverted to the wild-type phenotype implicates PvrR is inducing reversion from variant to wild-type phenotypes. These results indicated that PvrR may be acting upstream of the switch, since inactivation of pvrR by mutation was not found to result in conversion to the variant type.

Isolation of Additional Biofilm Regulator Genes

Based on the nucleotide and amino acid sequences described herein, the isolation and identification of additional coding sequences of genes regulating the formation of microbial biofilm is made possible using standard strategies and techniques that are well known in the art. For example, any microbe that possesses the ability to form a biofilm can serve as the nucleic acid source for the molecular cloning of such a gene, and these sequences are identified as ones encoding a protein exhibiting structures, properties, or activities associated with biofilm formation, such as the PvrR (FIG. 5D, SEQ ID NO:2), or any of the polynucleotides identified in the ORF1 (SEQ ID NOS:3 and 8-18) and ORF3 (SEQ ID NOS:5 and 30-34) regions.

In one particular example of such an isolation technique, any one of the nucleotide sequences described herein, including pvrR (FIG. 5A, SEQ ID NO:1), ORF1 (FIG. 5B, SEQ ID NO:3), or ORF3 (FIG. 5C, SEQ ID NO:5) may be used, together with conventional methods of nucleic acid hybridization screening. Such hybridization techniques and screening procedures are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. In one particular example, all or part of the pvrR, ORF1, or ORF3 sequences (described herein) may be used as a probe to screen a recombinant DNA library for genes having sequence identity to the pvrR, ORF1, or ORF3 genes. Hybridizing sequences are detected by plaque or colony hybridization according to standard methods.

Alternatively, using all or a portion of the amino acid sequences of PvrR, ORF1, or ORF3, one may readily design pvrR, ORF1, or ORF3 gene-specific oligonucleotide probes, including degenerate oligonucleotide probes (i.e., a mixture of all possible coding sequences for a given amino acid sequence). These oligonucleotides may be based upon the sequence of either DNA strand and any appropriate portion of the pvrR, ORF1, or ORF3 sequences. General methods for designing and preparing such probes are provided, for example, in Ausubel et al. (supra), and Berger and Kimmel, Guide to Molecular Cloning Techniques, 1987, Academic Press, New York. These oligonucleotides are useful for pvrR, ORF1, or ORF3 gene isolation, either through their use as probes capable of hybridizing to pvrR, ORF1, or ORF3 complementary sequences or as primers for various amplification techniques, for example, polymerase chain reaction (PCR) cloning strategies. If desired, a combination of different, detectably-labelled oligonucleotide probes may be used for the screening of a recombinant DNA library. Such libraries are prepared according to methods well known in the art, for example, as described in Ausubel et al. (supra), or they may be obtained from commercial sources.

As discussed above, sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., Proc. Natl. Acad. Sci. USA 85:8998, 1988.

Partial sequences, e.g., sequence tags, are also useful as hybridization probes for identifying full-length sequences, as well as for screening databases for identifying previously unidentified related virulence genes.

In general, the invention includes any nucleic acid sequence which may be isolated as described herein or which is readily isolated by homology screening or PCR amplification using any of the nucleic acid sequences disclosed herein such as those shown in FIGS. 5A, 5C, 5G, 6A-K, or 7A-7E.

It will be appreciated by those skilled in the art that as a result of the degeneracy of the genetic code, a multitude of polynucleotide sequences encoding PvrR, ORF1, or ORF3, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally-occurring pvrR, ORF1, or ORF3, and all such variations are to be considered as being specifically disclosed.

Although nucleotide sequences which encode PvrR, ORF1, ORF3, or their variants are preferably capable of hybridizing to the nucleotide sequence of the naturally-occurring pvrR, ORF1, or ORF3 under appropriately selected conditions of stringency, it may be advantageous to produce nucleotide sequences encoding PvrR, ORF1, ORF3, or their derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding PvrR, ORF1, ORF3, and their derivatives without altering the encoded amino acid sequences include the production of RNA transcripts having more desirable properties, such as a greater half-life, than transcripts produced from the naturally occurring sequence.

The invention also encompasses production of DNA sequences which encode PvrR, ORF1, ORF3, or fragments thereof generated entirely by synthetic chemistry. After production, the synthetic sequence may be inserted into any of the many available expression vectors and cell systems using reagents well known in the art. Moreover, synthetic chemistry may be used to introduce mutations into a sequence encoding any one of PvrR, ORF1, ORF3, or any fragment thereof.

Also encompassed by the invention are polynucleotide sequences that are capable of hybridizing to the claimed polynucleotide sequences, and, in particular, to those shown in FIG. 5A, 5B, 5C, 5G, 6A-6K, or 7A-7E and fragments thereof under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

Methods for DNA sequencing are well known in the art and may be used to practice any of the embodiments of the invention. The resulting sequences are analyzed using a variety of algorithms which are well known in the art. (See, e.g., Ausubel, F. M. (1997) Short Protocols in Molecular Biology, John Wiley & Sons, New York N.Y., unit 7.7).

Polypeptide Expression

In general, polypeptides of the invention (e.g., PvrR, ORF1, or ORF3 as shown in FIGS. 5D, 5E, 5F, 6L-6V, or 7F-7J) may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). Also included in the invention are polypeptides which are modified in ways which do not abolish their pathogenic activity (assayed, for example as described herein). Such changes may include certain mutations, deletions, insertions, or post-translational modifications, or may involve the inclusion of any of the polypeptides of the invention as one component of a larger fusion protein.

The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes fragments of any one of the polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events). The aforementioned general techniques of polypeptide expression and purification can also be used to produce and isolate useful peptide fragments or analogs (described herein).

Antibodies

The polypeptides disclosed herein or variants thereof or cells expressing them can be used as an immunogen to produce antibodies immunospecific for such polypeptides. “Antibodies” as used herein include monoclonal and polyclonal antibodies, chimeric, single chain, simianized antibodies and humanized antibodies, as well as Fab fragments, including the products of an Fab immunolglobulin expression library.

To generate antibodies, a coding sequence for a polypeptide of the invention may be expressed as a C-terminal fusion with glutathione S-transferase (GST) (Smith et al., Gene 67:31, 1988). The fusion protein is purified on glutathione-Sepharose beads, eluted with glutathione, cleaved with thrombin (at the engineered cleavage site), and purified to the degree necessary for immunization of rabbits. Primary immunizations are carried out with Freund's complete adjuvant and subsequent immunizations with Freund's incomplete adjuvant. Antibody titres are monitored by Western blot and immunoprecipitation analyses using the thrombin-cleaved protein fragment of the GST fusion protein. Immune sera are affinity purified using CNBr-Sepharose-coupled protein. Antiserum specificity is determined using a panel of unrelated GST proteins.

As an alternate or adjunct immunogen to GST fusion proteins, peptides corresponding to relatively unique immunogenic regions of a polypeptide of the invention may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides of the invention are prepared according to standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra). Once produced, monoclonal antibodies are also tested for specific recognition by Western blot or immunoprecipitation analysis (by the methods described in Ausubel et al., supra. Antibodies which specifically recognize the polypeptide of the invention are considered to be useful in the invention; such antibodies may be used, e.g., in an immunoassay. Alternatively monoclonal antibodies may be prepared using the polypeptide of the invention described above and a phage display library (Vaughan et al., Nature Biotech 14:309, 1996).

Preferably, antibodies of the invention are produced using fragments of the polypeptides disclosed herein which lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Antibodies against any of the polypeptides described herein may be employed to treat bacterial infections, for example, those infections involving biofilm formation. Thus, among others, antibodies against, for example, polypeptides of PvrR (SEQ ID NO: 2), ORF1 (SEQ ID NO: 4), or ORF3 (SEQ ID NO: 6) shown respectively in FIGS. 5D, E, or F may be employed to treat infections, particularly bacterial infections and especially chronic infections associated with CF or biofilm formation associated with indwelling medical devices, conjunctivitis, pneumonia, and bacteremia.

Diagnostics

In another embodiment, antibodies which specifically bind any of the polypeptides described herein may be used for the diagnosis of bacterial infection. A variety of protocols for measuring such polypeptides, including ELISAs, RIAs, and FACS, are known in the art and provide a basis for diagnosing bacterial infections.

In another aspect, hybridization with PCR probes which are capable of detecting polynucleotide sequences, including genomic sequences, encoding pvrR, ORF1, ORF3, or closely related molecules may be used to identify nucleic acid sequences which encode its gene product. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding PvrR, ORF1, or ORF3 allelic variants, or related sequences.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Microarrays may be prepared, used, and analyzed using methods known in the art. (See, e.g., Brennan et al., U.S. Pat. No. 5,474,796; Schena et al., Proc. Natl. Acad. Sci. 93:10614, 1996; Baldeschweiler et al., PCT application WO95/251116, 1995; Shalon, D. et al., PCT application WO95/35505, 1995; Heller et al., Proc. Natl. Acad. Sci. 94:2150, 1997; and Heller et al., U.S. Pat. No. 5,605,662.)

Screening Assays

As discussed above, we have identified a biofilm regulator gene, pvrR, of P. aeruginosa that mediates biofilm formation and antibiotic resistance by a microbe. Based on this discovery, we have developed screening assays for identifying compounds that enhance or inhibit the action of a polypeptide or the expression of a nucleic acid sequence of the invention. The method of screening may involve high-throughput techniques.

Any number of methods are available for carrying out such screening assays. In one working example, candidate compounds are added at varying concentrations to the culture medium of pathogenic cells expressing one of the nucleic acid sequences of the invention. Gene expression is then measured, for example, by standard Northern blot analysis (Ausubel et al., supra) or RT-PCR, using any appropriate fragment prepared from the nucleic acid molecule as a hybridization probe. The level of gene expression in the presence of the candidate compound is compared to the level measured in a control culture medium lacking the candidate molecule. A compound which promotes an increase in the expression of the pvrR gene or functional equivalent is considered useful in the invention; such a molecule may be used, for example, as a therapeutic to combat the pathogenicity of an infectious organism, for example, by decreasing its ability to form a biofilm and rendering it susceptible to antibiotic treatment.

In another working example, the effect of candidate compounds may be measured at the level of polypeptide production using the same general approach and standard immunological techniques, such as Western blotting or immunoprecipitation with an antibody specific for a biofilm regulator polypeptide, such as PvrR. For example, immunoassays may be used to detect or monitor the expression of at least one of the polypeptides of the invention in a microbial organism. Polyclonal or monoclonal antibodies (produced as described above) which are capable of binding to such a polypeptide may be used in any standard immunoassay format (e.g., ELISA, Western blot, or RIA assay) to measure the level of the polypeptide. A compound which promotes an increase in the expression of the polypeptide is considered particularly useful. Again, such a molecule may be used, for example, as a therapeutic to combat the biofilm formation of an organism as is described above.

In yet another working example, candidate compounds may be screened for those which specifically bind to and agonize a PvrR polypeptide (a polypeptide having the amino acid sequences shown in FIG. 5D) of the invention. The efficacy of such a candidate compound is dependent upon its ability to interact with the PvrR polypeptide or functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate biofilm formation may be assayed by any standard assay (e.g., those described herein).

In one particular working example, a candidate compound that binds to a polypeptide (e.g, PvrR) may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the pathogenicity polypeptide (e.g, biofilm regulator polypeptide) is identified on the basis of its ability to bind to the pathogenicity polypeptide (e.g, biofilm regulator polypeptide) and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to render a pathogen incapable of forming a biofilm (e.g., as described herein). Compounds isolated by this approach may also be used, for example, as therapeutics to treat or prevent the onset of a pathogenic infection, disease, or both. Compounds which are identified as binding to pathogenicity polypeptides (e.g, biofilm regulator polypeptides) with an affinity constant less than or equal to 10 mM are considered particularly useful in the invention.

Potential agonists include organic molecules, peptides, peptide mimetics, polypeptides, and antibodies that bind to a nucleic acid sequence or polypeptide of the invention (e.g, biofilm regulator polypeptides) and thereby increase its activity. Potential agonists also include small molecules that bind to and occupy the binding site of the polypeptide thereby preventing binding to cellular binding molecules, such that normal biological activity is prevented.

Compounds that decrease only antibiotic resistance of a microbe are also identified by monitoring reversion of bacterial colonies from the antibiotic resistant phenotype to the wild-type susceptible phenotype. In one working example, screens for compounds that increase reversion rate are conducted by plating antibiotic resistant variant bacteria on antibiotic-free media in the presence or absence of a candidate compound. The plates are then cultured using standard methods. The plates are then visually inspected for revertants, i.e., bacterial colonies having a wild-type phenotype. The number of wild-type phenotype bacterial colonies is compared between plates cultured in the presence or absence of a candidate compound. Compounds that increase the number of wild-type revertants, relative to the number of wild-type revertants on a control plate without the compound, are taken as useful in the invention.

Additionally, compounds that decrease antibiotic resistance are identified by monitoring for an increase in the susceptibility of bacteria to antibiotics. In yet another working example, compounds that decrease antibiotic resistance are identified by plating wild-type bacteria on antibiotic containing plates in the presence or absence of a candidate compound. The plates are cultured using standard methods, and then visually inspected for bacterial colonies. The number of antibiotic resistant bacterial colonies is compared between plates cultured in the presence or absence of a candidate compound. Compounds that decrease the number of antibiotic resistant variant colonies, relative to the number of antibiotic resistant variant colonies on a control plate without the compound, are taken as useful in the invention.

In another working example, a gene that regulates biofilm formation is identified by monitoring its activity or activity of its encoded polypeptide, when mutated. Bacteria are mutagenized using standard methods, such as transposon mutagenesis. Mutagenized and wild-type bacteria are then plated on antibiotic containing plates. These plates are cultured using standard methods, and then are visually inspected for the presence of antibiotic resistant variant bacteria. The number of antibiotic resistant variant bacterial colonies (e.g., small colony variants) is compared between mutagenized bacterial plates and wild-type control plates. This comparison is typically conducted when variant colonies begin to appear on the wild-type plate. A decrease or increase in the number of antibiotic resistant variant bacterial colonies (e.g., small colony variants) on a plate containing mutagenized bacteria is taken as an indication of the presence of a genetic mutation in a gene that regulates biofilm formation. The mutated gene is then identified according to standard methods.

In yet another working example, a gene that regulates biofilm or phenotype-mediated antibiotic resistance is identified as follows. For example, a candidate gene (e.g., as part of a genomic library) is introduced into a variant host cell (e.g., Pseudomonas aeruginosa PA14 RSCV). Next, the transformed host cell is monitored for reversion from the rough small colony variant phenotype to wild-type. The plates are then cultured using standard methods and monitored for the appearance of colonies with a wild-type phenotype. The number of wild-type phenotype bacterial colonies is then compared between plates containing transformants and variants carrying the vector alone. An increase in the number of wild-type revertants, relative to the number of wild-type revertants on a control plate with the vector alone, identifies a gene that regulates biofilm formation or phenotype-mediated antibiotic resistance. A gene identified using this method is subsequently isolated using standard procedures known in the art.

In another working example, small colony phenotypic variants are plated on an appropriate antibiotic medium (for example, those described herein) in the presence of a candidate compound and reversion to wild-type is monitored. Compounds that promote reversion from PA14 RSCV to wild-type are taken as being useful in the invention.

In another working example, a gene that regulates or is involved in phenotype-mediated or biofilm-mediated antibiotic resistance or biofilm formation is identified as follows. Bacteria are mutagenized using standard methods, such as transposon mutagenesis. Mutagenized bacteria are then plated on Trypticase Soy Agar (TSA) plates containing antibiotic. These plates are cultured using standard methods, and then inspected for bacterial growth. A decrease in the number of bacterial colonies or their absence on a mutagenized plate, relative to a control plate containing non-mutagenized bacteria identifies the presence of a genetic mutation in a gene that regulates phenotype-mediated or biofilm-mediated antibiotic resistance and biofilm formation. A gene identified using this method is subsequently isolated using standard procedures known in the art.

In another working example, a gene that regulates or is involved in phenotype-mediated or biofilm-mediated antibiotic resistance or biofilm formation is identified as follows. Bacteria are mutagenized using standard methods, such as transposon mutagenesis. Mutagenized bacteria are then transferred to Trypticase Soy Broth (TSB) liquid culture media containing an antibiotic. The bacteria are then cultured using standard methods, and the cultures are inspected for the presence of bacterial growth. Bacterial growth is compared between mutagenized cultures and wild-type control cultures. Bacterial growth can be identified, for example, by visual inspection, by measuring optical density at 600 nm, or by other standard methods. The inability of a mutant to grow in liquid culture with antibiotics indicates the presence of a genetic mutation in a gene that regulates or is involved in phenotype-mediated or biofilm-mediated antibiotic resistance and biofilm formation. A gene identified using this method is subsequently isolated using standard procedures known in the art.

In another working example, a gene that regulates or is involved in phenotype-mediated or biofilm-mediated antibiotic resistance or biofilm formation is identified as follows. Bacteria are mutagenized using standard methods, such as transposon mutagenesis. Mutagenized bacteria are then plated on TSA plates containing antibiotic. These plates are cultured using standard methods, and then inspected for bacterial growth. The inability of a mutant to grow in TSA supplemented with antibiotics is taken as an indication of the presence of a genetic mutation in a gene that regulates or is involved in phenotype-mediated or biofilm-mediated resistance and biofilm formation. A gene identified using this method is subsequently isolated using standard procedures known in the art.

In another working example, a gene that regulates or is involved in phenotype-mediated or biofilm-mediated antibiotic resistance or biofilm formation is identified as follows. Bacteria are mutagenized using standard methods, such as transposon mutagenesis. Mutagenized bacteria are then transferred to liquid culture media TSB containing an antibiotic. The bacteria are then cultured using standard methods, and the cultures are inspected for the presence of bacterial growth. Bacterial growth is compared between mutagenized cultures and wild-type control cultures. Bacterial growth can be identified, for example, by visual inspection, by measuring optical density at 600 nm, or by other standard methods. The inability of a mutant to grow in liquid culture with antibiotics indicates the presence of a genetic mutation in a gene that regulates or is involved in phenotype-mediated or biofilm-mediated antibiotic resistance and biofilm formation. A gene identified using this method is subsequently isolated using standard procedures known in the art.

Each of the DNA sequences provided herein may also be used in the discovery and development of antipathogenic compounds (e.g., antibiotics). The encoded protein, upon expression, can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The antagonists and agonists of the invention may be employed, for instance, to inhibit and treat a variety of bacterial infections, for example, those involving biofilm formation.

Optionally, compounds identified in any of the above-described assays may be confirmed as useful in conferring protection against the development of a pathogenic infection in any standard animal model (e.g., the mouse-burn assay described herein) and, if successful, may be used as anti-pathogen therapeutics (e.g, antibiotics).

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of reducing pathogenic virulence (e.g., reducing biofilm formation) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have an anti-pathogenic or anti-virulence activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Pharmaceutical Therapeutics

The invention provides a simple means for identifying compounds (including peptides, small molecule inhibitors, and mimetics) capable of inhibiting the pathogenicity (e.g., biofilm formation) of a pathogen. Accordingly, a chemical entity discovered to have medicinal value using the methods described herein is useful as a drug or as information for structural modification of existing anti-pathogenic compounds, e.g., by rational drug design. Such methods are useful for screening compounds having an effect on a variety of pathogens that form biofilms including, but not limited to, bacteria. Examples of pathogenic bacteria include, without limitation, Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Enterococcus, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

For therapeutic uses, the compositions or agents identified using the methods disclosed herein may be administered systemically, for example, formulated in a pharmaceutically-acceptable buffer such as physiological saline. Treatment may be accomplished directly, e.g., by treating the animal with antagonists which disrupt, suppress, attenuate, or neutralize the biological events associated with a pathogenicity polypeptide (e.g., a biofilm regulator polypeptide). Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of an anti-pathogenic agent in a physiologically-acceptable carrier. Suitable carriers and their formulation are described, for example, in Remington's Pharmaceutical Sciences by E. W. Martin. The amount of the anti-pathogenic agent (e.g., an antibiotic) to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation (e.g., biofilm formation). If desired, such treatment is also performed in conjunction with standard antibiotic therapy.

### Other Embodiments

In general, the invention includes any nucleic acid sequence which may be isolated as described herein or which is readily isolated by homology screening or PCR amplification using the nucleic acid sequences of the invention. Also included in the invention are polypeptides which are modified in ways which do not abolish their pathogenic activity (assayed, for example as described herein). Such changes may include certain mutations, deletions, insertions, or post-translational modifications, or may involve the inclusion of any of the polypeptides of the invention as one component of a larger fusion protein. Also, included in the invention are polypeptides that have lost their pathogenicity.

Thus, in other embodiments, the invention includes any protein which is substantially identical to a polypeptide of the invention. Such homologs include other substantially pure naturally-occurring polypeptides as well as allelic variants; natural mutants; induced mutants; proteins encoded by DNA that hybridizes to any one of the nucleic acid sequences of the invention under high stringency conditions or, less preferably, under low stringency conditions (e.g., washing at 2×SSC at 40° C. with a probe length of at least 40 nucleotides); and proteins specifically bound by antisera of the invention.

The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids.

In addition to full-length polypeptides, the invention also includes fragments of any one of the polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Furthermore, the invention includes nucleotide sequences that facilitate specific detection of any of the nucleic acid sequences of the invention. Thus, for example, nucleic acid sequences described herein or fragments thereof may be used as probes to hybridize to nucleotide sequences by standard hybridization techniques under conventional conditions. Sequences that hybridize to a nucleic acid sequence coding sequence or its complement are considered useful in the invention. Sequences that hybridize to a coding sequence of a nucleic acid sequence of the invention or its complement and that encode a polypeptide of the invention are also considered useful in the invention. As used herein, the term “fragment,” as applied to nucleic acid sequences, means at least 5 contiguous nucleotides, preferably at least 10 contiguous nucleotides, more preferably at least 20 to 30 contiguous nucleotides, and most preferably at least 40 to 80 or more contiguous nucleotides. Fragments of nucleic acid sequences can be generated by methods known to those skilled in the art.

The invention further provides a method for inducing an immunological response in an individual, particularly a human, which includes inoculating the individual with, for example, any of the polypeptides (or a fragment or analog thereof or fusion protein) of the invention to produce an antibody and/or a T cell immune response to protect the individual from infection, especially bacterial infection (e.g., a Pseudomonas aeruginosa infection). The invention further includes a method of inducing an immunological response in an individual which includes delivering to the individual a nucleic acid vector to direct the expression of a polypeptide described herein (or a fragment or fusion thereof) in order to induce an immunological response.

The invention also includes vaccine compositions including the polypeptides or nucleic acid sequences of the invention. For example, the polypeptides of the invention may be used as an antigen for vaccination of a host to produce specific antibodies which protect against invasion of bacteria. The invention therefore includes a vaccine formulation which includes an immunogenic recombinant polypeptide of the invention together with a suitable carrier.

The invention further provides compositions (e.g., nucleotide sequence probes), polypeptides, antibodies, and methods for the diagnosis of a pathogenic condition.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

1. An isolated polypeptide comprising an amino acid sequence having at least 50% identity to the amino acid sequence of PvrR (SEQ ID NO:2), wherein expression of said polypeptide, in a microorganism, affects phenotype-mediated antibiotic-resistance in said microorganism.

2. The isolated polypeptide of claim 1, said polypeptide comprising the amino acid sequence of PvrR (SEQ ID NO:2).

3. The isolated polypeptide of claim 1, wherein said amino acid sequence consists essentially of the amino acid sequence of PvrR (SEQ ID NO:2) or a fragment thereof.

4. An isolated polypeptide fragment of the isolated polypeptide of claim 1.

5. The isolated polypeptide fragment of claim 4, wherein said polypeptide fragment comprises 200 contiguous amino acids of SEQ ID NO:2.

6. An isolated polynucleotide having at least 50% identity to the nucleotide sequence of pvrR (SEQ ID NO:1), wherein expression of said polynucleotide, in a microorganism, affects phenotype-mediated antibiotic-resistance in said microorganism.

7. The isolated polynucleotide of claim 6, said polynucleotide comprising the nucleotide sequence of pvrR (SEQ ID NO:1) or a complement thereof.

8. The isolated polynucleotide of claim 7, said polynucleotide consisting essentially of the nucleotide sequence of pvrR (SEQ ID NO:1) or a fragment thereof.

9. A vector comprising the isolated polynucleotide of any one of claims 6, 7, or 8.

10. A host cell comprising the vector of claim 9.

11. A screening method for identifying a compound that modulates gene expression of a regulator polynucleotide that affects phenotype-mediated antibiotic-resistance in a microorganism, said method comprising the steps of:
(a) providing a microbial cell comprising a polynucleotide having at least 50% identity to the nucleotide sequence of pvrR (SEQ ID NO:1), wherein expression of said polynucleotide, in said microbial cell, affects phenotype-mediated antibiotic-resistance in said microbial cell;
(b) contacting said microbial cell with a compound; and
(c) comparing the level of gene expression of said polynucleotide in the presence of said compound with the level of gene expression in the absence of said compound; wherein a measurable difference in gene expression indicates that said compound modulates gene expression of a regulator polynucleotide that affects phenotype-mediated antibiotic-resistance in a microorganism.

12. The method of claim 11, wherein said screening method identifyies a compound that increases transcription of said regulator polynucleotide.

13. The method of claim 11, wherein said screening method identifies a compound that decreases transcription of said regulator polynucleotide.

14. The method of claim 11, wherein said screening method identifies a compound that increases translation of an mRNA transcribed from said regulator polynucleotide.

15. The method of claim 11, wherein said screening method identifies a compound that decreases translation of an mRNA transcribed from said regulator polynucleotide.

16. The method of claim 11, wherein the compound is a member of a chemical library.

17. The method of claim 11, wherein said microbial cell belongs to the genus Pseudomonas, Vibrio, Salmonella, or Staphylococcus.

18. The method of claim 11, wherein said microbial cell is a phenotypic variant having increased biofilm formation.

19. The method of claim 18, wherein said phenotypic variant is a small colony variant.

20. The method of claim 19, wherein said small colony variant is a small colony variant of Pseudomonas, Vibrio, Salmonella, or Staphylococcus.

21. The method of claim 18, wherein said small colony variant is a rough small colony variant.

22. The method of claim 21, wherein said rough small colony variant is Pseudomonas, Vibrio, or Salmonella.

23. The method of claim 11, wherein the activity of the compound is dependent upon the presence of the pvrR gene (SEQ ID NO:1) or a functional equivalent thereof.

24. The method of claim 11, wherein said compound targets the pvrR gene (SEQ ID NO:1) or a functional equivalent thereof.

25. The method of claim 11, wherein expression of said polynucleotide mediates phenotypic switching of said microbial cell in the presence of a high concentration of an antibiotic.

26. The method of claim 11, wherein said polypeptide is expressed by the isolated polynucleotide of any one of claims 6, 7, or 8.

27. A screening method for identifying a compound that modulates an activity of a polypeptide that affects phenotype-mediated antibiotic-resistance in a microorganism, said method comprising the steps of:
(a) providing a microbial cell expressing a polypeptide having at least 50% identity to the amino acid sequence of PvrR (SEQ ID NO:2), wherein expression of said polypeptide, in said microbial cell, affects phenotype-mediated antibiotic-resistance in said microbial cell;
(b) contacting said microbial cell with a compound; and
(c) comparing an activity of said polypeptide in the presence of said compound with said activity in the absence of said compound; wherein a measurable difference in the activity indicates that said compound modulates said activity of said polypeptide that affects phenotype-mediated antibiotic-resistance in a microorganism.

28. The method of claim 27, wherein said screening method identifies a compound that increases the activity of said polypeptide.

29. The method of claim 27, wherein said screening method identifies a compound that decreases the activity of said polypeptide.

30. The method of claim 27, wherein the compound is a member of a chemical library.

31. The method of claim 27, wherein comparing the activity of the polypeptide involves an immunological assay.

32. The method of claim 27, wherein said microbial cell belongs to the genus Pseudomonas, Vibrio, Salmonella, or Staphylococcus.

33. The method of claim 27, wherein said microbial cell is a phenotypic variant having increased biofilm formation.

34. The method of claim 33, wherein said phenotypic variant is Pseudomonas aeruginosa PA14 RSCV.

35. The method of claim 27, wherein said regulator polypeptide is the isolated polypeptide of claim 1.

36. The method of claim 27, wherein the activity of the polypeptide regulates phenotypic switching.

37. The method of claim 27, wherein the activity of the polypeptide regulates biofilm-mediated antibiotic-resistance.

38. The method of claim 27, wherein the activity of the polypeptide affects susceptibility of the microbial cell to antibiotic treatment.

39. The method of claim 27, wherein said polypeptide is an element of a two-component regulatory system.

40. The method of claim 27, wherein the activity of the compound is dependent upon the presence of the PvrR polypeptide (SEQ ID NO:2) or a functional equivalent thereof.

41. The method of claim 27, wherein said compound targets the PvrR polypeptide (SEQ ID NO:2) or a functional equivalent thereof.

42. The method of claim 27, wherein said polypeptide mediates phenotypic switching of said microbial cell in the presence of a high concentration of an antibiotic.

43. The method of claim 27, wherein said polypeptide is expressed by the isolated polynucleotide of any one of claims 6, 7, or 8.

44. A screening method for identifying a compound that modulates microbial biofilm formation, said method comprising the steps of:
(a) culturing a microbial cell comprising a polypeptide having at least 50% identity to the amino acid sequence of PvrR (SEQ ID NO:2), wherein said microbial cell, upon culturing, forms a biofilm;
(b) contacting said microbial cell with a compound; and
(c) comparing microbial biofilm formation in the presence of said compound with microbial biofilm formation in the absence of said compound; wherein a measurable difference in said microbial biofilm formation indicates that said compound modulates biofilm formation.

45. The method of claim 44, wherein said screening method identifies a compound that increases biofilm formation.

46. The method of claim 44, wherein said screening method identifies a compound that decreases biofilm formation.

47. The method of claim 44, wherein biofilm formation is measured by assaying microbial aggregation.

48. The method of claim 47, wherein microbial aggregation is assayed using a microscope.

49. The method of claim 47, wherein microbial aggregation is assayed using a salt aggregation test.

50. The method of claim 47, wherein microbial aggregation is assayed using an attachment assay.

51. The method of claim 44, wherein the compound is a member of a chemical library.

52. The method of claim 44, wherein said microbial cell belongs to the genus Pseudomonas, Vibrio, Salmonella, or Staphylococcus.

53. The method of claim 44, wherein said microbial cell is a phenotypic variant having increased biofilm formation.

54. The method of claim 53, wherein said phenotypic variant is a small colony variant.

55. The method of claim 54, wherein said small colony variant is a small colony variant of Pseudomonas, Vibrio, Salmonella, or Staphylococcus.

56. The method of claim 54, wherein said small colony variant is a rough small colony variant.

57. The method of claim 56, wherein said rough small colony variant is Pseudomonas, Vibrio, or Salmonella.

58. The method of claim 44, wherein the activity of the compound is dependent upon the presence of PvrR polypeptide (SEQ ID NO: 2) or a functional equivalent thereof.

59. The method of claim 44, wherein said compound targets the PvrR polypeptide (SEQ ID NO:2) or a functional equivalent thereof.

60. The method of claim 44, wherein expression of said polypeptide mediates phenotypic switching of said microbial cell in the presence of a high concentration of an antibiotic.

61. The method of claim 44, wherein said polypeptide is an isolated polypeptide of any one of claims 1, 2, or 3.

62. A method of treating a microbial infection involving a microorganism that forms a biofilm in a mammal, said method comprising administering to said mammal a therapeutically-effective amount of a compound that induces the expression of or activity of or represses the expression of or activity of the polypeptide of any one of claims 1, 2, or 3.

63. The method of claim 62, wherein said method further comprises administering to said mammal a therapeutically-effective amount of an antibiotic.

64. The method of claim 62, wherein said mammal is a human.

65. The method of claim 62, wherein said human has cystic fibrosis.

66. The method of claim 62, wherein said human has a chronic infection.

67. The method of claim 62, wherein the said microorganism belongs to the genus Pseudomonas, Vibrio, Salmonella or Staphylococcus.

68. A method of cleaning or disinfecting a surface at least partially covered by a microorganism that forms a biofilm, said method comprising contacting said microorganism with a cleaning composition comprising a compound that induces the expression of or activity of or represses the expression of or activity of the polypeptide of claim 1, 2, or 3.

69. The method of claim 68, wherein said microorganism belongs to the genera Pseudomonas, Vibrio, Salmonella or Staphylococcus.

70. A screening method for identifying a compound that decreases pathogenicity of an antibiotic-resistant phenotypic variant, said method comprising the steps of:
(a) contacting an antibiotic-resistant phenotypic variant with a candidate compound; and
(b) measuring reversion of said antibiotic-resistant phenotypic variant to a wild-type phenotype, an increase in reversion indicating that said compound decreases pathogenicity of said antibiotic-resistant phenotypic variant.

71. The method of claim 70, wherein said antibiotic-resistant phenotypic variant is a bacterial variant.

72. The method of claim 71, wherein said antibiotic-resistant phenotypic bacterial variant is cultured in the absence of an antibiotic.

73. The method of claim 71, wherein said antibiotic-resistant phenotypic bacterial variant has increased biofilm formation.

74. The method of claim 71, wherein said antibiotic-resistant phenotypic bacterial variant is a rough small colony variant.

75. The method of claim 71, wherein said antibiotic-resistant phenotypic bacterial variant is a hyperpiliated variant.

76. The method of claim 71, wherein said antibiotic-resistant phenotypic bacterial variant has increased hydrophobicity.

77. The method of claim 71, wherein said antibiotic-resistant phenotypic bacterial variant has an alteration in a surface component.

78. The method of claim 71, wherein said antibiotic-resistant phenotypic bacterial variant is a pathogen.

79. The method of claim 78, wherein said pathogen is a Gram positive bacterium.

80. The method of claim 79, wherein said pathogen is Staphylococcus.

81. The method of claim 78, wherein said pathogen is a Gram negative bacterium.

82. The method of claim 75, wherein said pathogen is Vibrio, Pseudomonas, or Salmonella.

83. A screening method for identifying a compound that decreases pathogenicity of a wild-type microbe, said method comprising the steps of:
(a) culturing a wild-type microbe with a candidate compound in the presence of an antibiotic; and
(b) comparing the number of antibiotic-resistant phenotypic variants in the presence of said compound to the number of antibiotic-resistant phenotypic variants in the absence of said compound, a decrease in the number of said antibiotic-resistant phenotypic variants in the presence of said compound indicating that said compound decreases pathogenicity of said wild-type microbe.

84. A screening method for identifying a polynucleotide encoding a regulator polypeptide that modulates an antibiotic-resistant phenotype of a microorganism, said method comprising the steps of:
(a) identifying an antibiotic-resistant phenotypic variant of a microorganism comprising a first phenotype;
(b) mutagenizing said antibiotic-resistant phenotypic variant of said microorganism, thereby generating a mutated phenotypic variant of said microorganism; and
(c) selecting said mutated phenotypic variant of step (b) having a second phenotype, other than the first phenotype of said antibiotic-resistant phenotypic variant, wherein said second phenotype identifies a mutation in said mutated phenotypic variant of step (b); and
(d) using said mutation for identifying a polynucleotide encoding a regulator polypeptide that modulates an antibiotic-resistant phenotype of a microorganism.

85. The method of claim 84, wherein said second phenotype comprises a wild-type phenotype.

86. A screening method for identifying a polynucleotide encoding a regulator polypeptide that modulates phenotype-mediated antibiotic-resistance of a microorganism, said method comprising the steps of:
(a) transforming an antibiotic-resistant phenotypic variant of a microorganism with a candidate polynucleotide encoding a regulator polypeptide; and
(b) culturing said transformed antibiotic-resistant phenotypic variant of a microorganism under conditions suitable for expression of said regulator polypeptide; and
(c) measuring reversion of said transformed antibiotic-resistant phenotypic variant of said microorganism to a wild-type phenotype, an increase in reversion identifies said polynucleotide as encoding a regulator polypeptide that modulates phenotype-mediated antibiotic-resistance.

87. The method of claim 80, wherein said polynucleotide encodes a regulator polypeptide that modulates a phenotypic switch from antibiotic-resistant phenotype to an antibiotic-susceptible phenotype.

88. The method of claim 80, wherein said polynucleotide having at least 50% identity to the nucleotide sequence of pvrR (SEQ ID NO:1) encodes an element of a two-component regulatory system.


SEQUENCE LISTING (39 sequences)

SEQ ID NO: 1 - DNA, 1200 bp, Pseudomonas aeruginosa PA14
atgagctggaaatcctatcgggtgctggtggtcgaagatcagccgtttcagcgcgaatacctgctcaacctgtttcgcgagcgcggcgtgcagtacctggtaggtgccggcgacggcgcggaggcgttgcgctgcctgaagcaggacaggttcgacctgatcctcagcgatctgatgatgccgggcatggatggtatccaaatgatcctgcaactgccgtatctcaagcatcgtccgaagctggcgctgatgagctcctcgtcgcagcggatgatgctcagtgccagccgggtcgcccagagtctcggcttgtcggtaatcgacctgttgcccaagccgactctgcccaaggccatcggccaacttctggaacacctggaaagatgcctcaggcagaagctggagccggaaaccgacgagactccgcatgggcgcacggcgttgctggatgccctgcataacgagcaactggtgacctggttccaggctaagaaatccctccacaccgggcgcatagtcggcgccgaggcgttgatacgctggagccacccgcagcatggcctgttgctgcccagctgtttcatgagtgatgtcgacgctaccggtctgcacgaggcgttgctctggcgcgtgctcgaacagaccctgaacgcccaggaatcgtggcgcagggcgggttacgagattccggtttcggtgaatctgccgccgcacctgctcgataaccaggaacttccggatcgactctatgagtacgtcggcgctcgcggggcttgtaccagctcactatgtttcgagttgaccgagagcagtgtcacaactctgtcaagtaactactatgcaggtgcctgtcgcttgcgcatgaaagggttcggattggcccaggacgactttggccagggttacagctcgttctataacctggtcacgacgcctttcacggagctgaagatcgaccgctccctagtccagggatgcgtagaggataacggcctcaatgcagctgtcatcagttgtattgagttgggtcaccgcctgaatctcgacgtggtggccgaaggcgtggagacctgcgaggaactgaatcttcttcgtcgtcttggctgcgaccgggcgcagggtttcctgatttctaaggcagtgtctgctcgtgagttcgagcggcagttaagggaggacggccccagcctccttgtttaa

SEQ ID NO: 2 - protein, 399 aa, Pseudomonas aeruginosa PA14
MSWKSYRVLVVEDQPFQREYLLNLFRERGVQYLVGAGDGAEALRCLKQDRFDLILSDLMMPGMDGIQMILQLPYLKHRPKLALMSSSSQRMMLSASRVAQSLGLSVIDLLPKPTLPKAIGQLLEHLERCLRQKLEPETDETPHGRTALLDALHNEQLVTWFQAKKSLHTGRIVGAEALIRWSHPQHGLLLPSCFMSDVDATGLHEALLWRVLEQTLNAQESWRRAGYEIPVSVNLPPHLLDNQELPDRLYEYVGARGACTSSLCFELTESSVTTLSSNYYAGACRLRMKGFGLAQDDFGQGYSSFYNLVTTPFTELKIDRSLVQGCVEDNGLNAAVISCIELGHRLNLDVVAEGVETCEELNLLRRLGCDRAQGFLISKAVSAREFERQLREDGPSLLV

SEQ ID NO: 3 - DNA, 1416 bp, Pseudomonas aeruginosa PA14
atgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 4 - protein, 471 aa, Pseudomonas aeruginosa PA14
MSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 5 - DNA, 1995 bp, Pseudomonas aeruginosa PA14
atgatggatgttatacgggagcatgaggtatttcttgggcgcatcgctcgaaaaagcgacaagaccacccagaagtacgactatgacgtggtgcctttgcagcggcacttgttggcaaaggaaaacggattagcggtctatgagggacgggagttttcctttgctatgccatttctactggctaccaagcacgcgttgagcgccgattcctcgggagatccgttttcgctcggtgtattgctcgccaatttctacggaagcttctggagtgtttccgcctatcccgcgccacagttactgatctttgatctttccggcagcacccgcctggcagtgccgtcgattccctccacagcgcagcgtgacaggttgagcggaagctatccgatgatagtcgagcgcattctggcgcgcttgcgcacccggccggtgggggaggacgctcagcgtgtccattggatacgcgctgatcgctatcgcgactcggcgctggagatgttgggagtcgcccgggttgatctgccggaaacactctggtggcacgacgagccgaaccatctgatcatcgctgcgagcctgcttgatctcaggcgaatcaatgacttcgaacagttggttgagcgcccggcattcgattcgtacagcctggtatcgccggatggcgaggtattgctcggcgcggcccctgcgaccggcctgagggatggcctgaacctcacccgacagggggtcgccgttcaactgcgcagccagcctgagaacggctggctcgcggtctaccgaaccgactacggcaatttctttcgccactcccggtggctggtggcaggtctgctgctgaccccggcgctgctcctggccggttggctcgggatgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatag

SEQ ID NO: 6 - protein, 664 aa, Pseudomonas aeruginosa PA14
MMDVIREHEVFLGRIARKSDKTTQKYDYDVVPLQRHLLAKENGLAVYEGREFSFAMPFLLATKHALSADSSGDPFSLGVLLANFYGSFWSVSAYPAPQLLIFDLSGSTRLAVPSIPSTAQRDRLSGSYPMIVERILARLRTRPVGEDAQRVHWIRADRYRDSALEMLGVARVDLPETLWWHDEPNHLIIAASLLDLRRINDFEQLVERPAFDSYSLVSPDGEVLLGAAPATGLRDGLNLTRQGVAVQLRSQPENGWLAVYRTDYGNFFRHSRWLVAGLLLTPALLLAGWLGMRWYTSSVVNPVHRAHRQLVESDTFSRTLIQTAPVALVVLTQDDQQLVTCNHLAAQWLGGPTEILGLTSNWKLFDARGQVPGDICIQVGGRYLQTAFAATRYAGTEAVLCVFNDITVHCEAETALSNAKRAADAASQAKTLFLARMSHEIRTPLYGVLGTLELLDLTTLNERQRAYLRTIQSSSATLMQLISDVLDVSKIEAGQMALTLAAFNPLDLVREVLGNFAASAMAKDLQVDPLDTLALEAQVAHGFEESVLFEVAGGSVGHFEEGVVGVVEQRLQRLFQLQRRLVAHLHEDDRQAPRSGVRRRLGSDPGQVHHIGIVLHRDSPATLAAAHGMAKIGHRGSIGVVRNVNFQASKTSIYIHYRDTFKSR

SEQ ID NO: 7 - DNA, 6410 bp, Pseudomonas aeruginosa PA14
cttcctcaccgcactttcattcaccgcaccgttatcggcgtcatggacaatgccctgcagtcctgagacctcattaggaatcatctgatagagggaatttttccatgccgtcagtctttcggcatgaagttgaagaatttcttacagccttttgatagcggtttctccactccgagtgctgcgctcaagctgctccgcatgctcggtggcgccttgatgttgtgcgtgctatgcagcctgatattcagtgtgagcatggttttaaaccatcaggtgtccctcagtcggcaagctatgaatgtggctatgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatgagctggaaatcctatcgggtgctggtggtcgaagatcagccgtttcagcgcgaatacctgctcaacctgtttcgcgagcgcggcgtgcagtacctggtaggtgccggcgacggcgcggaggcgttgcgctgcctgaagcaggacaggttcgacctgatcctcagcgatctgatgatgccgggcatggatggtatccaaatgatcctgcaactgccgtatctcaagcatcgtccgaagctggcgctgatgagctcctcgtcgcagcggatgatgctcagtgccagccgggtcgcccagagtctcggcttgtcggtaatcgacctgttgcccaagccgactctgcccaaggccatcggccaacttctggaacacctggaaagatgcctcaggcagaagctggagccggaaaccgacgagactccgcatgggcgcacggcgttgctggatgccctgcataacgagcaactggtgacctggttccaggctaagaaatccctccacaccgggcgcatagtcggcgccgaggcgttgatacgctggagccacccgcagcatggcctgttgctgcccagctgtttcatgagtgatgtcgacgctaccggtctgcacgaggcgttgctctggcgcgtgctcgaacagaccctgaacgcccaggaatcgtggcgcagggcgggttacgagattccggtttcggtgaatctgccgccgcacctgctcgataaccaggaacttccggatcgactctatgagtacgtcggcgctcgcggggcttgtaccagctcactatgtttcgagttgaccgagagcagtgtcacaactctgtcaagtaactactatgcaggtgcctgtcgcttgcgcatgaaagggttcggattggcccaggacgactttggccagggttacagctcgttctataacctggtcacgacgcctttcacggagctgaagatcgaccgctccctagtccagggatgcgtagaggataacggcctcaatgcagctgtcatcagttgtattgagttgggtcaccgcctgaatctcgacgtggtggccgaaggcgtggagacctgcgaggaactgaatcttcttcgtcgtcttggctgcgaccgggcgcagggtttcctgatttctaaggcagtgtctgctcgtgagttcgagcggcagttaagggaggacggccccagcctccttgtttaacgcagtatccccattatcgcggagtcgatcgcagccaaccaccgtcagcgcaacagtgtcgctgaaggaggccgcatcccgtgaagtctgctagtgccttggagcacgacaacaaacttttgctcaaatggacaaccctctcgcagagcctgagcatcggcttgatctgtgtggtggtgctgaccgtattgctgttcagcatctgttactggtcgctggggagattgtttcaggaggaggaggacaaagtctccttccacttcacccgtatgatggatgttatacgggagcatgaggtatttcttgggcgcatcgctcgaaaaagcgacaagaccacccagaagtacgactatgacgtggtgcctttgcagcggcacttgttggcaaaggaaaacggattagcggtctatgagggacgggagttttcctttgctatgccatttctactggctaccaagcacgcgttgagcgccgattcctcgggagatccgttttcgctcggtgtattgctcgccaatttctacggaagcttctggagtgtttccgcctatcccgcgccacagttactgatctttgatctttccggcagcacccgcctggcagtgccgtcgattccctccacagcgcagcgtgacaggttgagcggaagctatccgatgatagtcgagcgcattctggcgcgcttgcgcacccggccggtgggggaggacgctcagcgtgtccattggatacgcgctgatcgctatcgcgactcggcgctggagatgttgggagtcgcccgggttgatctgccggaaacactctggtggcacgacgagccgaaccatctgatcatcgctgcgagcctgcttgatctcaggcgaatcaatgacttcgaacagttggttgagcgcccggcattcgattcgtacagcctggtatcgccggatggcgaggtattgctcggcgcggcccctgcgaccggcctgagggatggcctgaacctcacccgacagggggtcgccgttcaactgcgcagccagcctgagaacggctggctcgcggtctaccgaaccgactacggcaatttctttcgccactcccggtggctggtggcaggtctgctgctgaccccggcgctgctcctggccggttggctcgggatgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatagagggttttcctatagacatgactctcttcac

SEQ ID NO: 8 - DNA, 2796 bp, Pseudomonas aeruginosa PA14
atgaagttgaagaatttcttacagccttttgatagcggtttctccactccgagtgctgcgctcaagctgctccgcatgctcggtggcgccttgatgttgtgcgtgctatgcagcctgatattcagtgtgagcatggttttaaaccatcaggtgtccctcagtcggcaagctatgaatgtggctatgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 9 - DNA, 2721 bp, Pseudomonas aeruginosa PA14
atgctcggtggcgccttgatgttgtgcgtgctatgcagcctgatattcagtgtgagcatggttttaaaccatcaggtgtccctcagtcggcaagctatgaatgtggctatgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 10 - DNA, 2703 bp, Pseudomonas aeruginosa PA14
atgttgtgcgtgctatgcagcctgatattcagtgtgagcatggttttaaaccatcaggtgtccctcagtcggcaagctatgaatgtggctatgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 11 - DNA, 2664 bp, Pseudomonas aeruginosa PA14
atggttttaaaccatcaggtgtccctcagtcggcaagctatgaatgtggctatgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 12 - DNA, 2625 bp, Pseudomonas aeruginosa PA14
atgaatgtggctatgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 13 - DNA, 2613 bp, Pseudomonas aeruginosa PA14
atgtacgaagcgcagctttatttcgagcagcgcgaggcgttgctcaatcacttgagcggcaatgtcgtgcccttggccgcgggtagagcgctcgtcaacgaagcgccgaacaatgtgagcatcctgccgttgagtgacggagggcgaggtctgctattgaccgctcgcacgctcggtgatctccgggaaaagcggctggcactgatgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 14 - DNA, 2409 bp, Pseudomonas aeruginosa PA14
atgtatctggtcgataccgacaaaggccctctggtttaccggcttaccgccgatggtaggccctcggcagcgatatccagcacgataaccaaagaggtgtaccgagccttgctggcgactccgtcggcgcctgttcactgggtgactgacggtggtacccctcaacggctgtacctttttgaatccttaggcgatgagccgggcgaggggtggctaggcctggagattctcggcgaagacctcgattcgatgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 15 - DNA, 2160 bp, Pseudomonas aeruginosa PA14
atgttgcgccggaatgatgccggaaactacatgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 16 - DNA, 2130 bp, Pseudomonas aeruginosa PA14
atgctgctggatcagcatgggcaggtcgtactcgctacggacgcagaggcgctggggagcggtgcgtcgcggacgcttttgcgtggagacggcttcggtttcatcggtgctggcccactgccgcagcatatggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 17 - DNA, 2001 bp, Pseudimonas aeruginosa PA14
atggtgcttttccagcacgtggggtcttcgagctgggatctgatctatcacatcggtatcggtcgcctgttgctggctctgtggctccctctgttacttgcctctgcgttggcactcgcagtcggcatcctactgcattggctggtgcggagcatcgagcgacgcttgatagagcccgcaaagcgacgccttgaagcattgaaggagagcgaagccttttcccgtgcagttatccaggccgcgcccgtcgcgctgtgcgtgctgcgtcgtgccgacgccgcagtggtcctggaaaatccccaggcgcgccaatggctgggtgatagcgaggcgattgcccacgacgcgccgagatggatttcccaggcgttcgcaggaggtgtgaagtgttctggagaagaactggaaaccgaggcagggctacatcttcatctcaattacacgcccacccgctataacggtgaagacgtattgttctgcgccttcagtgaaatcagtgcacgcaagcggatggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 18 - DNA, 1491 bp, Pseudomonas aeruginosa PA14
atggaggcggaactggctcgcgcaaaatccctggcggatgctgccaatgaagccaagacgctgtttctcgccaccatgagccatgaaatccgcacacctctgtacggcatgcttggcacgcttgagctgcttgggcgtaccgagctgagtcggcagcaggccggttacctaaaggcaatccagcattcctcgtcgaccctgctgcaactgatcagcgatgtgcttgacgtatccaagatagaggccggccaactggacctagagtgcgtggaattctccccgctggaattgaccgaagaggtcgtgcagtcgttcaccggtgccgcgcaggccaaggggctgcagttgtatacctgcctctctgcggagctgccgctgcgcatgcggggggccgcggcgtcgatccggcagattctcaacaacctgctgagcaacgcggtgaagttcaccgacaatggctatgtcaacgtccacctgaaggccagcgtggtcgatgccgaatgtgtgatgctgacctggcaggtcaacgataccggcatggggatcaacgtcgaggatcagccgcgtctgttcgaaccgttctaccagatacgccgctccgagcatccggtcgcaggcacgggcctcggcttgtcgatcagccagcgcctggcgcagctaatgaatggcagtctgaaactggtcagtgagctggggttgggcagcagctttagcctcaggcttccgcttgagcggatcgcgatgcaggctgagccgcaggacctagccgggtgcgccgtccaagtgctggcgcctgtccgcgacctaacggaatgcctgtgtggctggatctcccgctggggtggaagggccatggtcgcgacgccgaggtcgctggacgaggcggacgcgacctcgctgctggtcgaagtgttactgctggagggggcgccgatgttcgaagcatggccaggatgccgggtggagctttcccctcagggtgatatggagccgcaggcacagggccgcgactggctgctcgggctcaacaacctggacggcctgcatcgtgctctgggcctggcccatgggcgtctcgctgatccttcgacgccgccgatacggctggctccgttgcgcaatctaggtctccgcgtcctagtggtggaggataacgcgatcaaccagttgatcttgagggaccagatggaagcgctgggctgcagcgtggagctgctcttcgatggtcgcgaggcgttgctgcactgccagacggcctgcttcgacgtggtgctcaccgatatcaacatgccgaacatgaacggatacgagctaaccgcggagctacggcgccaagggttccggcagccgatcatcggcgcgacggcgaacgccatgcgtgaggagcgcgagcgctgcatgtccgccgggatgaacgattgcctggtcaaaccggtggatctgaatgcccttcagaactgcttgattaatattctcaaggtggatcgatga

SEQ ID NO: 19 - protein, 931 aa, Pseudomonas aeruginosa PA14
MKLKNFLQPFDSGFSTPSAALKLLRMLGGALMLCVLCSLIFSVSMVLNHQVSLSRQAMNVAMYEAQLYFEQREALLNHLSGNVVPLAAGRALVNEAPNNVSILPLSDGGRGLLLTARTLGDLREKRLALMYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 20 - protein, 906 aa, Pseudomonas aeruginosa PA14
MLGGALMLCVLCSLIFSVSMVLNHQVSLSRQAMNVAMYEAQLYFEQREALLNHLSGNVVPLAAGRALVNEAPNNVSILPLSDGGRGLLLTARTLGDLREKRLALMYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 21 - protein, 900 aa, Pseudomonas aeruginosa PA14
MLCVLCSLIFSVSMVLNHQVSLSRQAMNVAMYEAQLYFEQREALLNHLSGNVVPLAAGRALVNEAPNNVSILPLSDGGRGLLLTARTLGDLREKRLALMYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 22 - protein, 887 aa, Pseudomonas aeruginosa PA14
MVLNHQVSLSRQAMNVAMYEAQLYFEQREALLNHLSGNVVPLAAGRALVNEAPNNVSILPLSDGGRGLLLTARTLGDLREKRLALMYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 23 - protein, 874 aa, Pseudomonas aeruginosa PA14
MNVAMYEAQLYFEQREALLNHLSGNVVPLAAGRALVNEAPNNVSILPLSDGGRGLLLTARTLGDLREKRLALMYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 24 - protein, 870 aa, Pseudomonas aeruginosa PA14
MYEAQLYFEQREALLNHLSGNVVPLAAGRALVNEAPNNVSILPLSDGGRGLLLTARTLGDLREKRLALMYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 25 - protein, 802 aa, Pseudomonas aeruginosa PA14
MYLVDTDKGPLVYRLTADGRPSAAISSTITKEVYRALLATPSAPVHWVTDGGTPQRLYLFESLGDEPGEGWLGLEILGEDLDSMLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 26 - protein, 719 aa, Pseudomonas aeruginosa PA14
MLRRNDAGNYMLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 27 - protein, 709 aa, Pseudomonas aeruginosa PA14
MLLDQHGQVVLATDAEALGSGASRTLLRGDGFGFIGAGPLPQHMVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 28 - protein, 666 aa, Pseudomonas aeruginosa PA14
MVLFQHVGSSSWDLIYHIGIGRLLLALWLPLLLASALALAVGILLHWLVRSIERRLIEPAKRRLEALKESEAFSRAVIQAAPVALCVLRRADAAVVLENPQARQWLGDSEAIAHDAPRWISQAFAGGVKCSGEELETEAGLHLHLNYTPTRYNGEDVLFCAFSEISARKRMEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 29 - protein, 496 aa, Pseudomonas aeruginosa PA14
MEAELARAKSLADAANEAKTLFLATMSHEIRTPLYGMLGTLELLGRTELSRQQAGYLKAIQHSSSTLLQLISDVLDVSKIEAGQLDLECVEFSPLELTEEVVQSFTGAAQAKGLQLYTCLSAELPLRMRGAAASIRQILNNLLSNAVKFTDNGYVNVHLKASVVDAECVMLTWQVNDTGMGINVEDQPRLFEPFYQIRRSEHPVAGTGLGLSISQRLAQLMNGSLKLVSELGLGSSFSLRLPLERIAMQAEPQDLAGCAVQVLAPVRDLTECLCGWISRWGGRAMVATPRSLDEADATSLLVEVLLLEGAPMFEAWPGCRVELSPQGDMEPQAQGRDWLLGLNNLDGLHRALGLAHGRLADPSTPPIRLAPLRNLGLRVLVVEDNAINQLILRDQMEALGCSVELLFDGREALLHCQTACFDVVLTDINMPNMNGYELTAELRRQGFRQPIIGATANAMREERERCMSAGMNDCLVKPVDLNALQNCLINILKVDR

SEQ ID NO: 30 - DNA, 1992 bp, Pseudomonas aeruginosa PA14
atggatgttatacgggagcatgaggtatttcttgggcgcatcgctcgaaaaagcgacaagaccacccagaagtacgactatgacgtggtgcctttgcagcggcacttgttggcaaaggaaaacggattagcggtctatgagggacgggagttttcctttgctatgccatttctactggctaccaagcacgcgttgagcgccgattcctcgggagatccgttttcgctcggtgtattgctcgccaatttctacggaagcttctggagtgtttccgcctatcccgcgccacagttactgatctttgatctttccggcagcacccgcctggcagtgccgtcgattccctccacagcgcagcgtgacaggttgagcggaagctatccgatgatagtcgagcgcattctggcgcgcttgcgcacccggccggtgggggaggacgctcagcgtgtccattggatacgcgctgatcgctatcgcgactcggcgctggagatgttgggagtcgcccgggttgatctgccggaaacactctggtggcacgacgagccgaaccatctgatcatcgctgcgagcctgcttgatctcaggcgaatcaatgacttcgaacagttggttgagcgcccggcattcgattcgtacagcctggtatcgccggatggcgaggtattgctcggcgcggcccctgcgaccggcctgagggatggcctgaacctcacccgacagggggtcgccgttcaactgcgcagccagcctgagaacggctggctcgcggtctaccgaaccgactacggcaatttctttcgccactcccggtggctggtggcaggtctgctgctgaccccggcgctgctcctggccggttggctcgggatgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatag

SEQ ID NO: 31 - DNA, 1830 bp, Pseudomonas aeruginosa PA14
atgccatttctactggctaccaagcacgcgttgagcgccgattcctcgggagatccgttttcgctcggtgtattgctcgccaatttctacggaagcttctggagtgtttccgcctatcccgcgccacagttactgatctttgatctttccggcagcacccgcctggcagtgccgtcgattccctccacagcgcagcgtgacaggttgagcggaagctatccgatgatagtcgagcgcattctggcgcgcttgcgcacccggccggtgggggaggacgctcagcgtgtccattggatacgcgctgatcgctatcgcgactcggcgctggagatgttgggagtcgcccgggttgatctgccggaaacactctggtggcacgacgagccgaaccatctgatcatcgctgcgagcctgcttgatctcaggcgaatcaatgacttcgaacagttggttgagcgcccggcattcgattcgtacagcctggtatcgccggatggcgaggtattgctcggcgcggcccctgcgaccggcctgagggatggcctgaacctcacccgacagggggtcgccgttcaactgcgcagccagcctgagaacggctggctcgcggtctaccgaaccgactacggcaatttctttcgccactcccggtggctggtggcaggtctgctgctgaccccggcgctgctcctggccggttggctcgggatgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatag

SEQ ID NO: 32 - DNA, 1608 bp, Pseudomonas aeruginosa PA14
atgatagtcgagcgcattctggcgcgcttgcgcacccggccggtgggggaggacgctcagcgtgtccattggatacgcgctgatcgctatcgcgactcggcgctggagatgttgggagtcgcccgggttgatctgccggaaacactctggtggcacgacgagccgaaccatctgatcatcgctgcgagcctgcttgatctcaggcgaatcaatgacttcgaacagttggttgagcgcccggcattcgattcgtacagcctggtatcgccggatggcgaggtattgctcggcgcggcccctgcgaccggcctgagggatggcctgaacctcacccgacagggggtcgccgttcaactgcgcagccagcctgagaacggctggctcgcggtctaccgaaccgactacggcaatttctttcgccactcccggtggctggtggcaggtctgctgctgaccccggcgctgctcctggccggttggctcgggatgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatag

SEQ ID NO: 33 - DNA, 1500 bp, Pseudomonas aeruginosa PA14
atgttgggagtcgcccgggttgatctgccggaaacactctggtggcacgacgagccgaaccatctgatcatcgctgcgagcctgcttgatctcaggcgaatcaatgacttcgaacagttggttgagcgcccggcattcgattcgtacagcctggtatcgccggatggcgaggtattgctcggcgcggcccctgcgaccggcctgagggatggcctgaacctcacccgacagggggtcgccgttcaactgcgcagccagcctgagaacggctggctcgcggtctaccgaaccgactacggcaatttctttcgccactcccggtggctggtggcaggtctgctgctgaccccggcgctgctcctggccggttggctcgggatgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatag

SEQ ID NO: 34 - DNA, 1122 bp, Pseudomonas aeruginosa PA14
atgcgttggtacaccagcagcgtcgtcaacccggtgcatcgggcgcaccggcaactggtggagagcgacaccttcagccggacgctgatacagaccgcgccggtggctctggtggtgctgacccaggatgaccagcaactggtgacctgcaaccacttggccgcccagtggctgggcgggcccacggagatccttgggctgacttccaactggaagcttttcgatgcgcgtgggcaggtaccaggagacatctgtatccaggtcggtgggcgctatttgcagaccgccttcgcggcgacccgctatgccggcaccgaggcggtactgtgcgtattcaacgacatcacggtccactgcgaggcggagaccgcgctgtccaatgcgaagcgagcagcggatgccgccagccaggccaagaccctgttcctggcccgcatgagccatgaaatccgtactcccctgtacggtgtccttggcaccctggagttgctcgacctgaccaccctgaacgagcggcaacgcgcctacctacgcaccatccagagttcgtctgcgacgctcatgcaactgattagcgatgtgctggatgtctcgaagatcgaagcggggcagatggctctgaccctggccgccttcaatccgctggacctagtgcgggaagtgcttggcaactttgccgccagcgccatggccaaggacctgcaggtagacccgctcgatactcttgcgcttgaggcgcaggtcgcgcatggcttcgaagaaagcgttctgttcgaggttgctggtggctcggtcggccatttcgaagagggtgtcgtcggcgttgtcgaacaacgcctgcaacgcctgtttcagctgcagcgccgccttgtcgcgcacctgcacgaggatgaccggcaggcgccccgctccggcgttcggcgacggctcggaagcgaccctggtcaggtgcaccacattggcatcgttctgcatcgggactctcctgccaccctcgcggccgcgcatggaatggcaaaaatcgggcacagaggatcgattggcgtcgtccgtaacgtcaatttccaggcgtcaaaaacaagtatctacattcattatagagatactttcaaatctagatag

SEQ ID NO: 35 - protein, 663 aa, Pseudomonas aeruginosa PA14
MDVIREHEVFLGRIARKSDKTTQKYDYDVVPLQRHLLAKENGLAVYEGREFSFAMPFLLATKHALSADSSGDPFSLGVLLANFYGSFWSVSAYPAPQLLIFDLSGSTRLAVPSIPSTAQRDRLSGSYPMIVERILARLRTRPVGEDAQRVHWIRADRYRDSALEMLGVARVDLPETLWWHDEPNHLIIAASLLDLRRINDFEQLVERPAFDSYSLVSPDGEVLLGAAPATGLRDGLNLTRQGVAVQLRSQPENGWLAVYRTDYGNFFRHSRWLVAGLLLTPALLLAGWLGMRWYTSSVVNPVHRAHRQLVESDTFSRTLIQTAPVALVVLTQDDQQLVTCNHLAAQWLGGPTEILGLTSNWKLFDARGQVPGDICIQVGGRYLQTAFAATRYAGTEAVLCVFNDITVHCEAETALSNAKRAADAASQAKTLFLARMSHEIRTPLYGVLGTLELLDLTTLNERQRAYLRTIQSSSATLMQLISDVLDVSKIEAGQMALTLAAFNPLDLVREVLGNFAASAMAKDLQVDPLDTLALEAQVAHGFEESVLFEVAGGSVGHFEEGVVGVVEQRLQRLFQLQRRLVAHLHEDDRQAPRSGVRRRLGSDPGQVHHIGIVLHRDSPATLAAAHGMAKIGHRGSIGVVRNVNFQASKTSIYIHYRDTFKSR

SEQ ID NO: 36 - protein, 609 aa, Pseudomonas aeruginosa PA14
MPFLLATKHALSADSSGDPFSLGVLLANFYGSFWSVSAYPAPQLLIFDLSGSTRLAVPSIPSTAQRDRLSGSYPMIVERILARLRTRPVGEDAQRVHWIRADRYRDSALEMLGVARVDLPETLWWHDEPNHLIIAASLLDLRRINDFEQLVERPAFDSYSLVSPDGEVLLGAAPATGLRDGLNLTRQGVAVQLRSQPENGWLAVYRTDYGNFFRHSRWLVAGLLLTPALLLAGWLGMRWYTSSVVNPVHRAHRQLVESDTFSRTLIQTAPVALVVLTQDDQQLVTCNHLAAQWLGGPTEILGLTSNWKLFDARGQVPGDICIQVGGRYLQTAFAATRYAGTEAVLCVFNDITVHCEAETALSNAKRAADAASQAKTLFLARMSHEIRTPLYGVLGTLELLDLTTLNERQRAYLRTIQSSSATLMQLISDVLDVSKIEAGQMALTLAAFNPLDLVREVLGNFAASAMAKDLQVDPLDTLALEAQVAHGFEESVLFEVAGGSVGHFEEGVVGVVEQRLQRLFQLQRRLVAHLHEDDRQAPRSGVRRRLGSDPGQVHHIGIVLHRDSPATLAAAHGMAKIGHRGSIGVVRNVNFQASKTSIYIHYRDTFKSR

SEQ ID NO: 37 - protein, 535 aa, Pseudomonas aeruginosa PA14
MIVERILARLRTRPVGEDAQRVHWIRADRYRDSALEMLGVARVDLPETLWWHDEPNHLIIAASLLDLRRINDFEQLVERPAFDSYSLVSPDGEVLLGAAPATGLRDGLNLTRQGVAVQLRSQPENGWLAVYRTDYGNFFRHSRWLVAGLLLTPALLLAGWLGMRWYTSSVVNPVHRAHRQLVESDTFSRTLIQTAPVALVVLTQDDQQLVTCNHLAAQWLGGPTEILGLTSNWKLFDARGQVPGDICIQVGGRYLQTAFAATRYAGTEAVLCVFNDITVHCEAETALSNAKRAADAASQAKTLFLARMSHEIRTPLYGVLGTLELLDLTTLNERQRAYLRTIQSSSATLMQLISDVLDVSKIEAGQMALTLAAFNPLDLVREVLGNFAASAMAKDLQVDPLDTLALEAQVAHGFEESVLFEVAGGSVGHFEEGVVGVVEQRLQRLFQLQRRLVAHLHEDDRQAPRSGVRRRLGSDPGQVHHIGIVLHRDSPATLAAAHGMAKIGHRGSIGVVRNVNFQASKTSIYIHYRDTFKSR

SEQ ID NO: 38 - protein, 499 aa, Pseudomonas aeruginosa PA14
MLGVARVDLPETLWWHDEPNHLIIAASLLDLRRINDFEQLVERPAFDSYSLVSPDGEVLLGAAPATGLRDGLNLTRQGVAVQLRSQPENGWLAVYRTDYGNFFRHSRWLVAGLLLTPALLLAGWLGMRWYTSSVVNPVHRAHRQLVESDTFSRTLIQTAPVALVVLTQDDQQLVTCNHLAAQWLGGPTEILGLTSNWKLFDARGQVPGDICIQVGGRYLQTAFAATRYAGTEAVLCVFNDITVHCEAETALSNAKRAADAASQAKTLFLARMSHEIRTPLYGVLGTLELLDLTTLNERQRAYLRTIQSSSATLMQLISDVLDVSKIEAGQMALTLAAFNPLDLVREVLGNFAASAMAKDLQVDPLDTLALEAQVAHGFEESVLFEVAGGSVGHFEEGVVGVVEQRLQRLFQLQRRLVAHLHEDDRQAPRSGVRRRLGSDPGQVHHIGIVLHRDSPATLAAAHGMAKIGHRGSIGVVRNVNFQASKTSIYIHYRDTFKSR

SEQ ID NO: 39 - protein, 373 aa, Pseudomonas aeruginosa PA14
MRWYTSSVVNPVHRAHRQLVESDTFSRTLIQTAPVALVVLTQDDQQLVTCNHLAAQWLGGPTEILGLTSNWKLFDARGQVPGDICIQVGGRYLQTAFAATRYAGTEAVLCVFNDITVHCEAETALSNAKRAADAASQAKTLFLARMSHEIRTPLYGVLGTLELLDLTTLNERQRAYLRTIQSSSATLMQLISDVLDVSKIEAGQMALTLAAFNPLDLVREVLGNFAASAMAKDLQVDPLDTLALEAQVAHGFEESVLFEVAGGSVGHFEEGVVGVVEQRLQRLFQLQRRLVAHLHEDDRQAPRSGVRRRLGSDPGQVHHIGIVLHRDSPATLAAAHGMAKIGHRGSIGVVRNVNFQASKTSIYIHYRDTFKSR